(19) United States
(12) Patent Application Publication
(10) Pub. No.: US 2025/0352469 A1
(43) Pub. Date: Nov. 20, 2025

(54) NOVEL COMPOSITIONS, DEVICES, AND METHODS

(71) Applicant: INTRA-CELLULAR THERAPIES, INC., Bedminster, NJ (US)

(72) Inventor: Peng LI, New Milford, NJ (US)

(21) Appl. No.: 19/209,781

(22) Filed: May 15, 2025

Related U.S. Application Data
(60) Provisional application No. 63/648,644, filed on May 16, 2024.

(51) Int. Cl. A61K 9/10; A61K 9/00; A61K 31/4985; A61K 47/22; A61K 47/34

(52) U.S. Cl. CPC A61K 9/10; A61K 9/0019; A61K 31/4985; A61K 47/22; A61K 47/34

(57) ABSTRACT

The present disclosure relates to injectable pharmaceutical compositions comprising lumateperone or deuterolumateperone or tetradeuterolumateperone, in free, or pharmaceutically acceptable salt form, dissolved or suspended in a nonaqueous liquid polymeric vehicle comprising a solvent, devices comprising such compositions, and methods of use thereof in the treatment or prophylaxis of disease.

### CROSS-REFERENCE TO RELATED APPLICATIONS

This application is non-provisional application filed under 35 U.S.C. § 111 (a), which claims priority to, and the benefit of, U.S. Provisional Application Ser. No. 63/648,644, filed on May 16, 2024, the contents of which are hereby incorporated by reference in its entirety.

### TECHNICAL FIELD

The present disclosure relates to injectable pharmaceutical compositions comprising lumateperone or deuterolumateperone or tetradeuterolumateperone, in free, or pharmaceutically acceptable salt form, dissolved or suspended in a nonaqueous liquid polymeric vehicle comprising a solvent, devices comprising such compositions, and methods of use thereof in the treatment or prophylaxis of disease.

### BACKGROUND OF THE INVENTION

The substituted heterocycle fused gamma-carbolines lumateperone (4-((6bR,10aS)-3-methyl-2,3,6b,9,10,10a-hexahydro-1H-pyrido[3′,4′: 4,5]pyrrolo[1,2,3-de]quinoxalin-8 (7H)-yl)-1-(4-fluorophenyl)-1-butanone) is known to be a serotonin receptor (5-HT_{2A}), dopamine receptor (D1 and/or D2), and serotonin transporter (SERT) ligand, which is useful in treating a variety of central nervous system disorders.

Lumateperone antagonizes the serotonin-2A (5-HT_{2A}) receptor, and/or modulates dopamine receptor signaling at the level of key intra-cellular phosphoproteins. This compound is principally known to be useful for the treatment of positive and negative symptoms of schizophrenia, depression (especially acute depression and bipolar depression), anxiety and traumatic disorders (including acute anxiety and post-traumatic stress disorder), and dementias (including Alzheimer's disease and the symptoms associated therewith). At dopamine D2 receptors, this compound has dual properties and acts as both a post-synaptic antagonist and a pre-synaptic partial agonist of the D2 receptor. It also stimulates phosphorylation of glutamatergic NMDA NR2B, or GluN2B, receptors in a mesolimbic specific manner. It is believed that this regional selectivity in the brain areas thought to mediate the efficacy of antipsychotic drugs, together with the serotonergic, glutamatergic, and dopaminergic interactions, may result in antipsychotic efficacy for positive, negative, affective and cognitive symptoms associated with schizophrenia. The compound also exhibits serotonin reuptake inhibition, providing antidepressant activity for the treatment of schizoaffective disorder, co-morbid depression, and/or as a stand-alone treatment for major depressive disorder. Lumateperone is also useful for the treatment of bipolar disorder and other psychiatric and neurodegenerative disorders, particularly behavioral disturbances associated with dementia, autism and other CNS diseases. These features may be able to improve the quality of life of patients with schizophrenia and enhance social function to allow them to more fully integrate into their families and their workplace. Lumateperone displays differential dose-dependent effects, selectively targeting the 5-HT_{2A} receptor at low doses, while progressively interacting with the D2 receptor at higher doses. As a result, at lower doses, it is useful in treating sleep, aggression and agitation. At a high dose, it can treat acute exacerbated and residual schizophrenia, bipolar disorders, and mood disorders.

Lumateperone, having the formula:

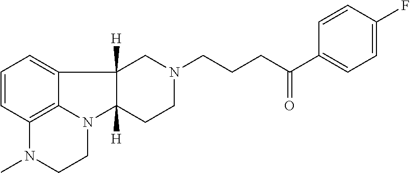 [SMILES: CN1CCN2c3c(cccc31)[C@@H]1CN(CCCC(=O)c3ccc(F)cc3)CC[C@@H]12]

is a novel therapeutic agent with potent (Ki-0.5 nM) 5-HT_{2A} receptor antagonism, activity as a mesolimbic/mesocortical-selective dopamine receptor protein phosphorylation modulator consistent with presynaptic D2 receptor partial agonism and postsynaptic D2 receptor antagonism (Ki=32 nM) in vivo, high D1 receptor affinity (Ki=52 nM), and inhibition of the serotonin transporter (SERT) (Ki=26-62 nM, using different assays for SERT activity). Lumateperone has recently been approved in the United States for the treatment of schizophrenia, and as a treatment for bipolar depression, and it is in clinical development as a treatment for agitation in dementia, including Alzheimer's Disease.

Lumateperone and related compounds have been disclosed in U.S. Pat. Nos. 6,548,493, 7,238,690, 6,552,017, 6,713,471, U.S. RE39,680, and U.S. RE39,679 (each of which are incorporated herein by reference) as novel compounds useful for the treatment of disorders associated with 5-HT2A receptor modulation such as anxiety, depression, psychosis, schizophrenia, sleep disorders, sexual disorders, migraine, conditions associated with cephalic pain, and social phobias.

U.S. Pat. No. 7,081,455, incorporated by reference herein, also disclose methods of making substituted heterocycle fused gamma-carbolines and uses of these gamma-carbolines as serotonin agonists and antagonists useful for the control and prevention of central nervous system disorders such as addictive behavior and sleep disorders.

U.S. Pat. Nos. 8,598,119, and 11,124,514, each incorporated herein by reference, disclose the use of specific substituted heterocycle fused gamma-carbolines for the treatment of a combination of psychosis and depressive disorders as well as sleep, depressive and/or mood disorders in patients with psychosis or Parkinson's disease and for the treatment or prophylaxis of disorders associated with dementia, particularly behavioral or mood disturbances such as agitation, irritation, aggressive/assaultive behavior, anger, physical or emotional outbursts and psychosis and sleep disorders associated with dementia.

U.S. Pat. No. 8,648,077, each incorporated herein by reference, disclose methods of preparing toluenesulfonic acid addition salt crystals of particular substituted heterocycle fused gamma-carbolines, e.g., toluenesulfonic acid addition salt of 4-((6bR, 10aS)-3-methyl-2,3,6b,9,10,10a-hexahydro-1H-pyrido[3′,4′: 4,5]pyrrolo[1,2,3-de]quinoxalin-8 (7H)-yl)-1-(4-fluorophenyl)-1-butanone. US 2020/0157100, disclosed herein by reference, similarly discloses various bis-tosylate salts of lumateperone.

U.S. Pat. Nos. 10,716,786 and 11,052,083, incorporated herein by reference, discloses subcutaneous, transmucosal and other dosage forms and administration routes for lumateperone and related compounds. US 2021/0315891, incorporated herein by reference, discloses transdermal dosage forms and administration routes for lumateperone and related compounds.

U.S. Pat. Nos. 10,695,345 and 11,052,084, and US 2021/0220280, each of which is incorporated herein by reference, discloses solid oral dosage forms for lumateperone, including tablets and capsules.

Deuterated lumateperone analogs, including deuterolumateperone and tetradeuterolumateperone, are disclosed in U.S. Pat. Nos. 10,077,267, 10,597,394, 10,688,097, 10,899,762, and 11,096,944, and patent publication US 2021/0008065, and US2023/0312573, the contents of each of which are hereby incorporated by reference in their entireties. As used herein, the term “deuterolumateperone” refers to 1-(4-fluoro-phenyl)-4-((6bR,10aS)-2,2-d₂-3-methyl-2,3,6b,9,10,10a-hexahydro-1H,7H-pyrido[3′,4′: 4,5]pyrrolo[1,2,3-de]quinoxalin-8-yl)-butan-1-one, which has also been referred to as “d₂-lumateperone” or “deulumateperone,” and which has the following structure:

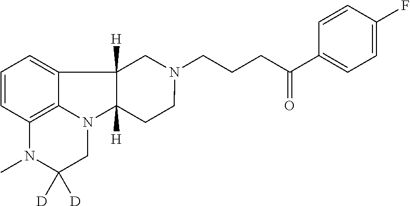 [SMILES: [2H]C1([2H])CN2c3c(cccc3N1C)[C@@H]1CN(CCCC(=O)c3ccc(F)cc3)CC[C@@H]12]

As used herein, the term “tetradeuterolumateperone” refers to 1-(4-fluoro-phenyl)-4-((6bR,10aS)-1,1,2,2-d₄-3-methyl-2,3,6b,9,10,10a-hexahydro-1H,7H-pyrido[3′,4′: 4,5]pyrrolo[1,2,3-de]quinoxalin-8-yl)-butan-1-one, which has also been referred to as “d₄-lumateperone,” and which has the following structure:

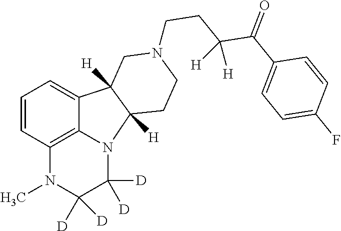 [SMILES: [2H]C1([2H])N(C)c2cccc3c2N([C@H]2CCN(CCCC(=O)c4ccc(F)cc4)C[C@@H]32)C1([2H])[2H]]

U.S. Pat. Nos. 8,993,572 and 9,371,324, each incorporated herein by reference, disclose prodrugs/metabolites of substituted heterocycle fused gamma-carboline for improved formulation, e.g., extended/controlled release formulation. This application discloses that heterocycle fused gamma-carboline N-substituted with a 4-fluorophenyl (4-hydroxy)butyl moiety are shown to have high selectivity for the serotonin transporter (SERT) relative to the heterocycle fused gamma-carboline containing 4-fluorophenylbutanone.

It has also recently been found that lumateperone may be particularly effective in treating acute depression and acute anxiety owing to its rapid onset of action compared to existing antidepressants, as disclosed in US 2021/0060009, incorporated herein by reference. This is believed to be due to it signaling through a neurotransmitter system separate from the traditional monoamine signaling systems. Lumateperone provides a dopamine D1 receptor-dependent enhancement of NMDA and AMPA currents coupled with activation of the mTOR (e.g., mTORC1) signaling pathway.

U.S. Pat. Nos. 9,708,322, 10,072,010, 10,472,359, 9,956,227, and 10,322,134, each of which is incorporated herein by reference, disclose and/or claim various long-acting injectable compositions comprising lumateperone and related compounds, as well as various polymeric matrix-based compositions (including PLGA-based compositions) comprising lumateperone, particularly in the form of microparticles or microspheres in an aqueous solvent.

However, it is particularly difficult to formulate a long-acting injectable (e.g., depot injection) composition, because of the need to provide a long-lasting, therapeutically effective, and non-toxic drug plasma level with minimal side effects. For example, for any long-acting injectable drug, there will be an optimal plasma concentration range which maximizes the balance between therapeutical efficacy and side effects, while there will also be a minimum therapeutically effective plasma concentration, and for some drugs, a maximum safe and tolerable plasma concentration. The goal of a long-acting injectable therapy is to maintain drug plasma levels within the optimal range for as long as possible, while avoiding plasma levels in above the maximum safe and tolerable value, and avoid plasma levels below the minimum efficacy value. In addition, many experimental long-acting injectable formulations suffer from the problem of “dose-dumping” in which a large an undesirable peak in plasma drug concentration occurs rapidly after injection, such as within the first 1-3 weeks. This can result in insufficient drug remaining in the depot for continued optimal drug release, while also potentially causing undesirable side effects or toxicities caused by the plasma concentration peak.

The plasma concentration profile of a long-acting injectable formulation can be difficult to predict, as it depends on numerous factors, including: the drug loading in the depot (i.e., concentration), the composition of the polymer such as lactide/glycolide ratio for PLGA polymers, the viscosity of the polymer matrix in which the drug is embedded, the degradation rate of the polymer matrix, the porosity of the polymer matrix, and for solid particles embedded in a polymer matrix, the particle size distribution of the particles. For a microsphere formulation, wherein the drug is embedded in polymer microspheres, the particle size distribution of the microspheres can also be important.

There remains a need for improved long-acting injectable formulations of lumateperone.

### BRIEF SUMMARY OF THE INVENTION

The present disclosure provides injectable pharmaceutical compositions comprising lumateperone or deuterolumateperone or tetradeuterolumateperone, in free or pharmaceutically acceptable salt form, dissolved or suspended in a nonaqueous liquid polymeric vehicle comprising a solvent. The present disclosure further provides a single-use kit, multiple-use kit, or pre-filled syringe (e.g., an automatic syringe) comprising any of such compositions. In some embodiments, the compositions are packaged in devices for injection, such as syringes, including pre-filled and/or automatic syringes. In particular embodiments, the syringes are designed for at-home use by patients and the syringes include automatically retracting needles. In some embodiments the compositions or composition component(s) may further comprise one or more additional therapeutic agents. The compositions and devices disclosed herein are useful for the treatment or prophylaxis of a variety of central nervous system disorders, such as schizophrenia and bipolar depression.

### DETAILED DESCRIPTION

Lumateperone is a novel therapeutic agent with potent (Ki=0.5 nM) 5-HT_{2A} receptor antagonism, activity as a mesolimbic/mesocortical-selective dopamine receptor protein phosphorylation modulator consistent with presynaptic D2 receptor partial agonism and postsynaptic D2 receptor antagonism (Ki=32 nM) in vivo, high D1 receptor affinity (Ki=52 nM), and inhibition of the serotonin transporter (SERT) (Ki=26-62 nM, using different assays for SERT activity). Lumateperone has recently been approved in the United States for the treatment of schizophrenia and bipolar depression, and it is in clinical development as a treatment for major depressive disorder. Deuterolumateperone is in clinical development as a treatment for agitation in dementia, including Alzheimer's Disease, and anxiety disorders.

In a first aspect, the present disclosure provides a nonaqueous injectable pharmaceutical composition (Composition 1), comprising lumateperone:

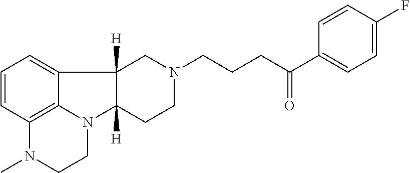 [SMILES: CN1CCN2c3c(cccc31)[C@@H]1CN(CCCC(=O)c3ccc(F)cc3)CC[C@@H]12]

in free or pharmaceutically acceptable salt form (e.g., in free base or tosylate salt form), wherein the lumateperone is dissolved or suspended in a nonaqueous liquid polymeric vehicle comprising at least one nonaqueous (e.g., organic) solvent, wherein the polymeric vehicle comprises at least one of a poly(lactic acid) polymer, a poly(glycolic acid) polymer, a poly(lactic-co-glycolic acid) copolymer, and a polyester-poly(ethylene glycol) copolymer, and wherein the composition does not comprise polymeric microspheres.

As used herein, the term “nonaqueous” means a composition, liquid, or solvent having less than 3% by weight of water, e.g., less than 2%, less than 1%, less than 0.5%, less than 0.1%, or less than 0.05%, by weight, Preferably, the compositions disclosed herein contain no added water, and preferably any organic solvents, including water-miscible organic solvents, are anhydrous (e.g., having water in less than 0.5%, less than 0.1%, or less than 0.05%, by weight).

It is understood that lactic acid and glycolic acid have the following structures:

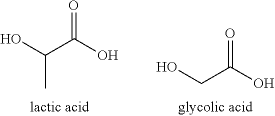

Poly(lactic acid) and poly(glycolic acid) are thus homopolymers of the polyester class:

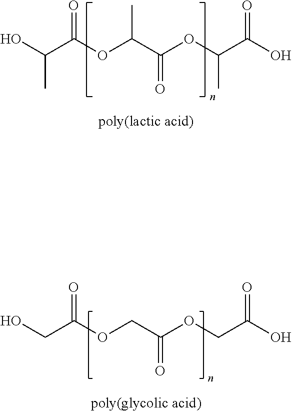

Poly(lactic-co-glycolic acid) copolymer is a copolymer consisting of both lactide units and glycolide units arranged in a linear fashion. It may have a random structure, in which the order of lactide and glycolide units is random throughout the length, or it may have a block structure, in which there are blocks of polylactide units and blocks of polyglycolide units. An example of a block copolymer structure would be:

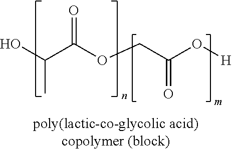

As shown in the above examples, poly(lactic acid), poly(glycolic acid), and poly(lactic-co-glycolic acid) copolymers all have a hydroxy terminal group at one end of the linear polymer, and a carboxyl terminal group at the opposite end. As such, it would be referred to as a polymer having an “acid end group.” Alternatively, the carboxylic acid end group may be reacted to form an ester, in which case the polymer is one having an “ester end group.” For example:

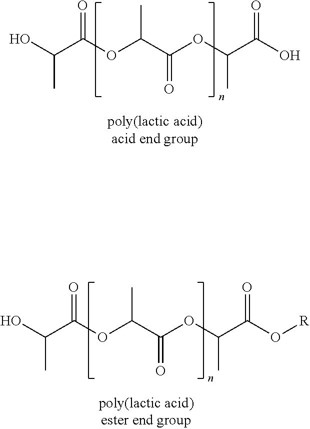

Ester end groups are preferably C1-6 alkyl esters, either linear or branched, in some embodiments, preferably linear C1-6 alkyl esters. For example, methyl, ethyl, propyl, isopropyl, n-butyl, s-butyl, isobutyl, tert-butyl, n-pentyl, s-pentyl, isopentyl, neopentyl, tert-pentyl, n-hexyl, s-hexyl, isohexyl, neohexyl, or tert-hexyl. Such ester end groups help promote a slower rate of in vivo degradation of the polymers and/or improve storage stability of the polymers.

As polyester polymers, these polymers comprise a large number of internal ester linkages. With an acid end group, the polymer may be slightly acidic, whereas with an ester end group, the polymer is neutral. However, it is understood that the use of the “-ic” suffix versus an “-ide” suffix in the polymer name, are generally interchangeable, and the use of the word “acid” in the polymer name does not necessarily imply the presence of an acid end group. Thus, for example, the terms “polylactic acid,” “poly(lactic acid),” “poly(lactide),” and “polylactide,” are to be considered synonymous, unless expressly indicated otherwise, and are all equivalent to the abbreviation PLA. Likewise, the terms “polyglycolic acid,” “poly(glycolic acid),” “poly(glycolide),” and “polyglycolide,” are to be considered synonymous, unless expressly indicated otherwise, and are all equivalent to the abbreviation PLG. Similarly, poly(lactic acid-co-glycolic acid), polylactic acid-co-glycolic acid, poly(lactide-co-glycolide), and polylactide-co-glycolide, are to be considered synonymous, unless expressly indicated otherwise, and are all equivalent to the abbreviation PLGA.

It is further understood that lactic acid is a chiral molecule because of the asymmetry of the central alpha carbon atom. Lactic acid therefore exists in two mirror image stereoisomeric forms (enantiomers), each of which can be referred to using varying alternative nomenclatures:

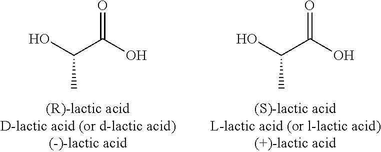

Generally, a PLA or PLGA polymer can be formed from either racemic lactic acid (resulting in equal amounts of D- and L-lactide units), or it can be formed from enantiomerically pure lactic acid (resulting in a polymer with only D- or only L-lactide units). When no label is used, such as in “poly(lactic acid)” or “PLGA” either racemic, enantiomerically pure, or any other combination of enantiomers is embraced. In contrast, poly(L-lactic acid) would refer to a polymer having only L-lactide units, and “poly(D,L-lactic acid)” would refer to a polymer having a racemic (equal) mixture of D- and L-lactide units. The equivalent applies to “poly(L-lactide-co-glycolide)” and “poly(D,L-lactide-co-glycolide),” for example. Poly(D,L-lactic acid) is commonly abbreviated as PDLLA, while poly(D,L-lactide-coglycolide) is commonly abbreviated as PLGA.

Lactic acid and glycolic acid can also be formed into copolymers or heteropolymers further comprising ethylene glycol, that is, polyester-poly(ethylene glycol) copolymers. As used herein, this term refers to any copolymer which comprises both poly(ethylene glycol) moieties and polyester moieties. This includes copolymers between lactic acid and ethylene glycol (poly(lactic acid)-PEG copolymers), copolymers between glycolic acid and ethylene glycol (poly(glycolic acid)-PEG copolymers), copolymers between lactic acid, glycolic acid, and ethylene glycol (poly(lactic acid)-poly(glycolic acid)-PEG copolymers. Such copolymers could be formed in a random arrangement or in a block arrangement, such as a deblock or triblock arrangement. As noted above, the lactic acid components of these polymers can also be racemic or enantiomerically pure (D- or L-lactic acid), and the carboxy terminal groups of the copolymers can be in their free acid forms or esterified. Thus, these polymers include both the diblock copolymer poly(D,L-lactide)-poly(ethylene glycol), also known as PDLLA-PEG, and the triblock copolymer poly(D,L-lactide)-co-poly(ethylene glycol)-co-poly(D,L-lactide), as known as PDLLA-PEG-PDLLA.

Two-component copolymers include, for example, the following, with the ratios between the monomer components determining the values of the integers n, m, n1, and/or n2:

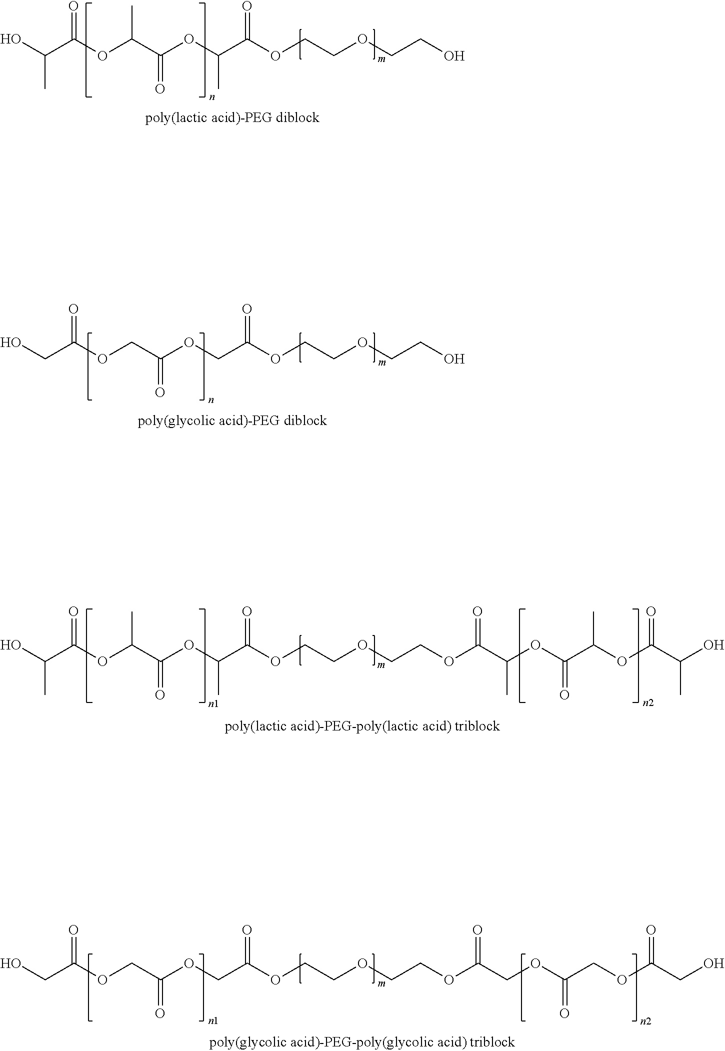

In some embodiments, the polyester-PEG copolymer (e.g., poly(lactic acid)-PEG copolymer) is a diblock copolymer wherein the free-hydroxy end of the PEG block is alkylated with a C1-6 alkyl group, either linear or branched, in some embodiments, preferably linear C1-6 alkyl groups. For example, methyl, ethyl, propyl, isopropyl, n-butyl, s-butyl, isobutyl, tert-butyl, n-pentyl, s-pentyl, isopentyl, neopentyl, tert-pentyl, n-hexyl, s-hexyl, isohexyl, neohexyl, or tert-hexyl. Such ether end groups help promote a slower rate of in vivo degradation of the polymers and/or improve storage stability of the polymers.

For example, Composition 1 may be as follows:
1.1. Composition 1, wherein the composition comprises the lumateperone in free base form (e.g., in free base solid amorphous dispersion form);
1.2. Composition 1, wherein the composition comprises lumateperone in pharmaceutically acceptable salt or co-crystal form;
1.3. Composition 1, wherein the composition comprises lumateperone in tosylate salt form, e.g., in one or more of mono-tosylate salt form, di-tosylate salt form, and tri-tosylate salt form;
1.4. Composition 1.3, wherein the composition comprises a combination of lumateperone in mono-tosylate salt form and lumateperone in di-tosylate salt form;
1.5. Any of Compositions 1 or 1.1-1.3, wherein the Composition comprises lumateperone in mono-tosylate salt form;
1.6. Composition 1.5, wherein the lumateperone mono-tosylate is in solid crystal form, e.g., having the physical and chemical properties as disclosed in U.S. Pat. No. 8,648,077, such as one or more of the XRPD spectrum, IR spectrum, and/or DSC/TGA spectrum as disclosed therein;
1.7. Composition 1, or any of 1.1-1.6, wherein the composition comprises the lumateperone (e.g., lumateperone free base or lumateperone tosylate) in an amount of 10 to 50% by weight of the composition, e.g., 10 to 40%, or 10 to 30%, or 10 to 25%, or 10 to 20%, or 10 to 15%, or 15 to 45%, or 15 to 40%, 15 to 35%, or 15 to 30%, or 15 to 25%, or 15 to 20%, or 20 to 45%, or 20 to 40%, or 20 to 35%, or 20 to 30%, or 20 to 25%, or 25 to 50%, or 25 to 45%, or 25 to 40%, or 25 to 35%, or 25 to 30%, or 30 to 40%, or about 15%, or about 20%, or about 25%, or about 30%, by weight of the composition, measured as the equivalent amount of free base lumateperone;
1.8. Composition 1, or any of 1.1-1.7, wherein the at least one nonaqueous solvent is a pharmacologically safe water-miscible organic solvent (e.g., N-methyl-2-pyrrolidone (NMP) or DMSO) or a pharmacologically safe triglyceride oil (e.g., a vegetable oil);
1.9. Composition 1.8, wherein the organic solvent is selected from N-methyl-2-pyrrolidone (NMP), dimethylsulfoxide (DMSO), dimethyl formamide (DMF), dimethylacetamide (DMA), 2-pyrrolidone, propylene carbonate, caprolactam, N-methylcaprolactam, and triacetin;
1.10. Composition 1.9, wherein the organic solvent is N-methyl-2-pyrrolidone (NMP);
1.11. Composition 1.8, wherein the triglyceride oil is selected from peanut oil, soybean oil, sesame oil, and castor oil;
1.12. Composition 1, or any of 1.1-1.11, wherein the composition comprises any one or more nonaqueous solvents (e.g., N-methyl-2-pyrrolidone or DMSO) in a net amount of 30 to 70% by weight of the composition, e.g., 35 to 70%, or 40 to 70%, or 45 to 70%, or 50 to 70%, or 30 to 60%, or 35 to 60%, or 40 to 60%, or 45 to 60% or 50 to 60%, or 30 to 55%, or 35 to 55%, or 40 to 55%, or 45 to 55%, or 50 to 55%, by weight of the composition;
1.13. Composition 1, or any of 1.1-1.12, wherein the polymeric vehicle comprises a poly(lactic acid) polymer (e.g., poly-1-lactide, poly-d-lactide, or poly-dl-lactide);
1.14. Composition 1, or any of 1.1-1.12, wherein the polymeric vehicle comprises a poly(glycolic acid) polymer;
1.15. Composition 1, or any of 1.1-1.12, wherein the polymeric vehicle comprises a poly(lactic-co-glycolic acid) copolymer (e.g., poly(l-lactide-co-glycolide), or poly(d-lactide-co-glycolide), or poly(dl-lactide-co-glycolide);
1.16. Composition 1.15, wherein the poly(lactic-co-glycolic acid) copolymer has a lactic to glycolic molar ratio of about 99:1 to 50:50, e.g., about 99:1 to 80:20, or about 99:1 to 90:10, or about 90:10 to 75:25, or about 90:10 to 80:20, or about 80:20 to 75:25, or about 75:25 to 50:50, or about 85:15 to 75:25, or about 70:30 to 60:40;
1.17. Composition 1.15, wherein the poly(lactic-co-glycolic acid) copolymer has a lactic to glycolic molar ratio of about 84:16 to 76:24, or about 73:27 to 52:48;
1.18. Composition 1.15, wherein the poly(lactic-co-glycolic acid) copolymer has a lactic to glycolic molar ratio of about 83:17 to 77:23, or about 70:30 to 55:45;
1.19. Composition 1.15, wherein the poly(lactic-co-glycolic acid) copolymer has a lactic to glycolic molar ratio of about 82:18 to 78:22, or about 68:32 to 58:42;
1.20. Composition 1.15, wherein the poly(lactic-co-glycolic acid) copolymer has a lactic to glycolic molar ratio of about 81:19 to 79:21, or about 68:32 to 62:38;
1.21. Composition 1.15, wherein the poly(lactic-co-glycolic acid) copolymer has a lactic to glycolic molar ratio of about 75:25, or about 80:20, or about 85:15, or about 90:10, or about 95:15, or about 98:2, or about 99:1;
1.22. Composition 1.15, wherein the poly(lactic-co-glycolic acid) copolymer has a lactic to glycolic molar ratio of about 80:20;
1.23. Composition 1.15, wherein the poly(lactic-co-glycolic acid) copolymer has a lactic to glycolic molar ratio of about 65:35;
1.24. Composition 1, or any of 1.1-1.23, wherein the at least one of a poly(lactic acid) polymer, a poly(glycolic acid) polymer, and a poly(lactic-co-glycolic acid) copolymer has an inherent viscosity of 0.1 to 0.9 dL/g, e.g., 0.1 to 0.75, or 0.1 to 0.7, or 0.6 to 0.6, or 0.1 to 0.5, or 0.1 to 0.4, or 0.1 to 0.3, or 0.1 to 0.2, or 0.15 to 0.45, or 0.15 to 0.4, or 0.15 to 0.3, or 0.15 to 0.25, or 0.15 to 0.2, or 0.2 to 0.8, or 0.2 to 0.6, or 0.2 to 0.4, or 0.2 to 0.3, or 0.25 to 0.5, or 0.25 to 0.4, or 0.25 to 0.35, or 0.25 to 0.3, or 0.3 to 0.6, or 0.3 to 0.5, or 0.3 to 0.45, or 0.3 to 0.4, or 0.3 to 0.35, or 0.5 to 0.8, or 0.5 to 0.75, or 0.5 to 0.7 dL/g;
1.25. Composition 1, or any of 1.1-1.24, wherein the at least one of a poly(lactic acid) polymer, a poly(glycolic acid) polymer, and a poly(lactic-co-glycolic acid) copolymer has an average molecular weight of 5,000 to 150,000 Daltons, e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
1.26. Composition 1, or any of 1.1-1.25, wherein the at least one of a poly(lactic acid) polymer, a poly(glycolic acid) polymer, and a poly(lactic-co-glycolic acid) copolymer has a molecular weight range of 5,000 to 150,000 Daltons (e.g., at least 90% of polymer molecules have a molecular weight within the range), e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
1.27. Composition 1, or any of 1.1-1.26, wherein the at least one of a poly(lactic acid) polymer, a poly(glycolic acid) polymer, and a poly(lactic-co-glycolic acid) copolymer has carboxylic acid end groups;
1.28. Composition 1, or any of 1.1-1.26, wherein the at least one of a poly(lactic acid) polymer, a poly(glycolic acid) polymer, and a poly(lactic-co-glycolic acid) copolymer has carboxylic ester end groups (e.g., such as a C1-6 linear ester end group, e.g., methyl, ethyl, propyl, isopropyl, butyl, pentyl, or hexyl ester end groups);
1.29. Composition 1, or any of 1.1-1.28, wherein the composition comprises any one or more a poly(lactic acid) polymer, a poly(glycolic acid) polymer, and a poly(lactic-co-glycolic acid) copolymer in a net amount of 10 to 60% by weight of the composition, e.g., 10 to 50%, or 10 to 45%, or 10 to 40%, or 15 to 50%, or 15 to 45%, or 15 to 40%, or 15 to 35%, or 20 to 50%, or 20 to 45%, or 20 to 40%, or 20 to 35%, or 25 to 50%, or 25 to 45%, or 25 to 40%, or 25 to 35%, or 30 to 50%, or 30 to 45%, or 30 to 40%, or 30 to 35%, or 35 to 50%, or 35 to 45%, or 35 to 40%, or about 30%, or about 35%, or about 45%, by weight of the composition;
1.30. Composition 1, or any of 1.1-1.29, wherein the polymeric vehicle comprises a polyester-poly(ethylene glycol) copolymer, such as poly(lactic acid)-PEG copolymer, poly(glycolic acid)-PEG copolymer, or a poly(lactic acid)-poly(glycolic acid)-PEG copolymer;
1.31. Composition 1.30, wherein said copolymer has a random arrangement;
1.32. Composition 1.30, wherein said copolymer has a block arrangement;
1.33. Composition 1.32, wherein said copolymer has a diblock (e.g., poly(lactic acid)-PEG, poly(glycolic acid)-PEG), or a triblock arrangement (e.g., poly(lactic acid)-PEG-poly(lactic acid), poly(glycolic acid)-PEG-poly(glycolic acid), poly(lactic acid)-PEG-poly(glycolic acid), poly(lactic acid)-poly(glycolic acid)-PEG copolymer, or poly(glycolic)-poly(lactic acid acid)-PEG copolymer;
1.34. Composition 1.33, wherein said copolymer is a poly(lactic acid)-PEG copolymer, optionally a diblock or triblock poly(lactic acid)-PEG copolymer;
1.35. Any of Compositions 1.30-1.34, wherein said copolymer has carboxylic acid end groups;
1.36. Any of Compositions 1.30-1.34, wherein said copolymer has carboxylic ester end groups (e.g., such as a C1-6 linear ester end group, e.g., methyl, ethyl, propyl, isopropyl, butyl, pentyl, or hexyl ester end groups);
1.37. Any of Compositions 1.30-1.36, wherein said copolymer is a diblock copolymer having an ether end group on the PEG block, e.g., a C1-6 linear ether end group, such as methyl, ethyl, propyl, isopropyl, butyl, pentyl, or hexyl ether end group);
1.38. Any of Compositions 1.30-1.37, wherein said copolymer has an inherent viscosity of 0.1 to 0.9 dL/g, e.g., 0.1 to 0.75, or 0.1 to 0.7, or 0.6 to 0.6, or 0.1 to 0.5, or 0.1 to 0.4, or 0.1 to 0.3, or 0.1 to 0.2, or 0.15 to 0.45, or 0.15 to 0.4, or 0.15 to 0.3, or 0.15 to 0.25, or 0.15 to 0.2, or 0.2 to 0.8, or 0.2 to 0.6, or 0.2 to 0.4, or 0.2 to 0.3, or 0.25 to 0.5, or 0.25 to 0.4, or 0.25 to 0.35, or 0.25 to 0.3, or 0.3 to 0.6, or 0.3 to 0.5, or 0.3 to 0.45, or 0.3 to 0.4, or 0.3 to 0.35, or 0.5 to 0.8, or 0.5 to 0.75, or 0.5 to 0.7 dL/g;
1.39. Any of Compositions 1.30-1.38, wherein said copolymer has an average molecular weight of 5,000 to 150,000 Daltons, e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
1.40. Any of Compositions 1.30-1.39, wherein said copolymer has a molecular weight range of 5,000 to 150,000 Daltons (e.g., at least 90% of polymer molecules have a molecular weight within the range), e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
1.41. Any of Compositions 1.30-1.40, wherein the composition comprises said copolymer in a net amount of 10 to 80% by weight of the composition, e.g., 10% to 75%, or 10% to 70%, or 10% to 65%, or 10% to 60%, or 10% to 55%, 10 to 50%, or 10 to 45%, or 10 to 40%, 15% to 75%, or 15% to 70%, or 15% to 65%, or 15% to 60%, or 15% to 55%, or 15 to 50%, or 15 to 45%, or 15 to 40%, or 15 to 35%, 20% to 75%, or 20% to 70%, or 20% to 65%, or 20% to 60%, or 20% to 55%, or 20 to 50%, or 20 to 45%, or 20 to 40%, or 20 to 35%, 25% to 75%, or 25% to 70%, or 25% to 65%, or 25% to 60%, or 25% to 55%, or 25 to 50%, or 25 to 45%, or 25 to 40%, or 25 to 35%, or 30% to 75%, or 30% to 70%, or 30% to 65%, or 30% to 60%, or 30% to 55%, or 30 to 50%, or 30 to 45%, or 30 to 40%, or 30 to 35%, or 35 to 50%, or 35 to 45%, or 35 to 40%, or about 30%, or about 35%, or about 45%, by weight of the composition;
1.42. Any of Compositions 1.30-1.41, wherein the composition comprises both a diblock polyester-poly(ethylene glycol) copolymer and a triblock polyester-poly(ethylene glycol) copolymer, each independently as described above, e.g., in a molar ratio from 6:1 to 1:6, e.g., 5:1 to 1:5, or 4:1 to 1:4, or 3:1 to 1:3, or 2:1 to 1:2, or about 4:1, or about 1:4, or about 3:1, or about 1:3, or about 2:1, or about 1:2, or about 1:1, or about 3:5, or about 5:3;
1.43. Any of Compositions 1.30-1.41, wherein the composition comprises both a diblock poly(lactic acid)-PEG copolymer and a triblock poly(lactic acid)-PEG copolymer, each independently as described above;
1.44. Composition 1.43, wherein the diblock poly(lactic acid)-PEG copolymer has a methyl ether end group (e.g., a methoxy-PEG block);
1.45. Composition 1, or any of 1.1-1.44, wherein the composition further comprises an anti-oxidant, e.g., selected from one or more of tocopherol, butylated hydroxytoluene (BHT), propyl gallate (PG, e.g., n-propyl gallate), ascorbic acid, butylated hydroxyanisole (BHA), tert-butylhydroquinone (TBHQ), carotenoids, glutathione, sodium metabisulfite, sodium ethylenediaminetetraacetate, cysteine, methionine, sesamol, citrate (e.g., sodium citrate), and citric acid, optionally in an amount of 0.1 to 1% by weight of the composition (e.g., about 0.25%, or about 0.5%, or about 0.75%);
1.46. Composition 1, or any of 1.1-1.44, wherein the composition further comprises an anti-oxidant selected from alpha-tocopherol, butylated hydroxytoluene (BHT), propyl gallate (PG, e.g., n-propyl gallate), and butylated hydroxyanisole (BHA), in an amount of 0.1 to 1% by weight of the composition (e.g., about 0.25%, or about 0.5%, or about 0.75%);
1.47. Composition 1, or any of 1.1-1.46, wherein the composition further comprises toluenesulfonic acid, e.g., in a molar ratio of about 1:1 to 1:2 with respect to the lumateperone mono-tosylate, e.g., 1:1 to 1:1.5 molar ratio, or 1:1 to 1:2 molar ratio, or about a 1:1 molar ratio;
1.48. Composition 1, or any of 1.1-1.47, wherein the composition further comprises one or more additional polymers selected from polysaccharides (e.g., starches, dextrans, pectins, alginates, carrageenans, cellulose), cellulose derivatives (e.g., carboxymethyl cellulose, methylcellulose, hydroxyalkyl celluloses (e.g., hydroxyethyl cellulose, hydroxypropyl cellulose, hydroxypropyl methyl cellulose), synthetic polymers (e.g., polyvinylpyrrolidones, polyethylene oxide and/or polypropylene oxide polymers and copolymers (e.g., poloxamers, such as poloxamer 188), polyacrylate polymers (e.g., carbopols), and polyamide polymers;
1.49. Composition 1.48, wherein the composition comprises one or more of said additional polymers each in an amount of 1 to 25% by weight of the composition, e.g., from 1 to 20%, or 1 to 15%, or 1 to 10%, or 1 to 5%, or 5 to 20%, or 5 to 15%, or 5 to 10%, or 10 to 20%, or 10 to 15%, or 15 to 20%, by weight of the composition;
1.50. Composition 1, or any of 1.1-1.49, wherein the composition further comprises one or more other excipients selected from inorganic or organic acids (e.g., citric acid, lactic acid, malic acid, gluconic acid, benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid) and their salts (e.g., sodium, potassium, calcium, magnesium, lithium, ammonium salts of aforementioned acids), inorganic or organic bases (e.g., alkali metal or alkaline earth metal carbonates, bicarbonates, hydroxide, oxides), anionic surfactants (e.g., sodium lauryl sulfate, sodium laureth sulfate, sodium dodecylbenzenesulfonate, sodium lauroyl sarcosinate, sodium stearate), cationic surfactants (e.g., benzalkonium halides, cetylpyridinium halides, cetrimonium halides, benzethonium halides), zwitterionic surfactants (e.g., cocamidoalkyl betaines, such as cocamidopropyl betaine), nonionic surfactants (e.g., fatty alcohol ethoxylates (e.g., polyethylene glycol polydodecyl ethers)), sorbitan esters (e.g., sorbitan monolaurate, sorbitan monooleate, sorbitan monopalmitate, sorbitan monostearate, sorbitan tristearate), and polyethoxylated sorbitan esters (e.g., polysorbate 20, polysorbate 40, polysorbate 60, polysorbate 80);
1.51. Composition 1.50, wherein the composition comprises one or more of said additional excipients each in an amount of 0.1 to 10% by weight of the composition, e.g., from 0.1 to 8%, or 0.1 to 6%, or 0.1 to 5%, or 0.1 to 3%, or 0.1 to 2.5%, or 0.1 to 2%, or 0.1 to 1.5%, or 0.1 to 1%, or 0.1 to 0.5%, by weight of the composition;
1.52. Composition 1, or any of 1.1-1.51, wherein the polymeric vehicle consists of the one or more of a poly(lactic acid) polymer, a poly(glycolic acid) polymer, and a poly(lactic-co-glycolic acid) copolymer and the solvent (e.g., N-methyl-2-pyrrolidone or DMSO);
1.53. Composition 1, or any of 1.1-1.52, wherein the composition consists essentially of lumateperone (e.g., lumateperone free base), the one or more of a poly(lactic acid) polymer, a poly(glycolic acid) polymer, and a poly(lactic-co-glycolic acid) copolymer and the solvent (e.g., N-methyl-2-pyrrolidone or DMSO), and optionally an anti-oxidant (e.g., n-propyl gallate);
1.54. Composition 1, or any of 1.1-1.52, wherein the composition consists essentially of lumateperone (e.g., lumateperone free base), poly(lactic acid) polymer and/or poly(lactic-co-glycolic acid) copolymer, and the solvent (e.g., N-methyl-2-pyrrolidone or DMSO), and optionally an anti-oxidant (e.g., n-propyl gallate);
1.55. Composition 1, or any of 1.1-1.52, wherein the composition consists essentially of lumateperone (e.g., lumateperone free base), poly(lactic acid) polymer, and the solvent (e.g., N-methyl-2-pyrrolidone or DMSO), and optionally an anti-oxidant (e.g., n-propyl gallate);
1.56. Composition 1, or any of 1.1-1.52, wherein the composition consists essentially of lumateperone (e.g., lumateperone free base), poly(lactic-co-glycolic acid) copolymer, and the solvent (e.g., N-methyl-2-pyrrolidone or DMSO), and optionally an anti-oxidant (e.g., n-propyl gallate);
1.57. Any one of Compositions 1.52-1.56, wherein the amount of lumateperone (e.g., lumateperone free base) is 10-40% by weight of the composition, e.g., 15%, 20%, 25%, 30%, or 35%;
1.58. Composition 1, or any of 1.1-1.57, wherein the composition consists essentially of 10-40% lumateperone (e.g., lumateperone free base), 15-50% poly(lactic acid) polymer and/or poly(lactic-co-glycolic acid) copolymer (e.g., PLGA 65:35 or 80:20), and balance solvent (e.g., N-methyl-2-pyrrolidone or DMSO), and optionally an anti-oxidant (e.g., 0.1-1% n-propyl gallate), each measured by weight of the composition;
1.59. Composition 1, or any of 1.1-1.57, wherein the composition consists essentially of 20-40% lumateperone (e.g., lumateperone free base), 20-30% poly(lactic acid) polymer and/or poly(lactic-co-glycolic acid) copolymer, and balance solvent (e.g., N-methyl-2-pyrrolidone or DMSO), and optionally an anti-oxidant (e.g., 0.1-1% n-propyl gallate), each measured by weight of the composition;
1.60. Composition 1, or any of 1.1-1.57, wherein the composition consists essentially of 20-30% lumateperone (e.g., lumateperone free base), 25-35% poly(lactic acid) polymer and/or poly(lactic-co-glycolic acid) copolymer, and balance solvent (e.g., N-methyl-2-pyrrolidone or DMSO), and optionally an anti-oxidant (e.g., n-propyl gallate), each measured by weight of the composition;
1.61. Composition 1.60, wherein the composition comprises about 20% lumateperone (e.g., lumateperone free base) and 28-32% polymer (e.g., about 30% polymer), or wherein the composition comprises about 25% lumateperone (e.g., lumateperone free base) and 30-35% polymer (e.g., about 33% polymer), or wherein the composition comprises about 30% lumateperone (e.g., lumateperone free base) and 25-30% polymer (e.g., about 27% polymer);
1.62. Any of Compositions 1.52-1.61, wherein the polymer has an inherent viscosity of 0.1 to 0.5 dl/g, e.g., 0.1 to 0.4 or 0.1 to 0.3, or 0.2 to 0.5, or 0.2 to 0.4;
1.63. Composition 1, or any of 1.1-1.62, wherein the composition has no added water and comprises not more than a trace amount of water (e.g., less than 1% by weight, or less than 0.5% by weight, or less than 0.1% by weight, or less than 0.05% by weight);
1.64. Composition 1, or any of 1.1-1.63, wherein the lumateperone (e.g., the lumateperone free base or lumateperone tosylate) is suspended in the nonaqueous liquid polymeric vehicle (e.g., to form an emulsion);
1.65. Composition 1, or any of 1.1-1.63, wherein the lumateperone (e.g., the lumateperone free base or lumateperone tosylate) is dissolved in the nonaqueous liquid polymeric vehicle (e.g., to form a solution, such as a homogenous solution);
1.66. Composition 1, or any of 1.1-1.65, wherein the composition is a suspension or emulsion;
1.67. Composition 1, or any of 1.1-1.65, wherein the composition is a solution, e.g., a homogenous solution;
1.68. Composition 1, or any of 1.1-1.67, wherein the composition has a viscosity of 1 to 100 centiPoise (cP), e.g., 1 to 75 cP, or 1 to 70 cP, or 1 to 65 cP, or 1 to 60 cP, or 1 to 50 cP, or 1 to 40 cP, or 1 to 30 cP, or 1 to 20 cP, or 1 to 15 cP, or 1 to 10 cP, or 5 to 60 cP, or 5 to 50 cP, or 5 to 40 cP, or 5 to 30 cP, or 5 to 20 cP, or 5 to 10 cP, or 10 to 60 cP, or 10 to 50 cP, or 10 to 40 cP, or 10 to 30 cP, or 10 to 20 cP, or 20 to 60 cP, or 20 to 50 cP, or 20 to 40 cP, or 20 to 30 cP, or 30 to 60 cP, or 30 to 50 cP, or 30 to 40 cP, or 30 to 60 cP, or 40 to 50 cP, or 50 to 60 cP;
1.69. Composition 1, or any of 1.1-1.68, wherein the composition has a viscosity of 50 to 2000 centiPoise (cP), e.g., 100 to 1100 cP, or 100 to 1000 cP, or 100 to 900 cP, or 100 to 800 cP, or 100 to 700 cP, or 100 to 600 cP, or 100 to 500 cP, or 100 to 400 cP, or 1 to 300 cP, or 1 to 200 cP, or 200 to 1000 cP, or 200 to 900 cP, or 200 to 800 cP, or 200 to 700 cP, or 200 to 600 cP, or 200 to 500 cP, or 200 to 400 cP, or 200 to 300 cP, or 300 to 1000 cP, or 300 to 900 cP, or 300 to 800 cP, or 300 to 700 cP, or 300 to 600 cP, or 300 to 500 cP, or 300 to 400 cP, or 400 to 1000 cP, or 400 to 900 cP, or 400 to 800 cP, or 400 to 700 cP, or 400 to 600 cP, or 400 to 500 cP, or 500 to 1000 cP, or 500 to 900 cP, or 500 to 800 cP, or 500 to 700 cP, or 500 to 600 cP, or 600 to 1000 cP, or 600 to 900 cP, or 600 to 800 cP, or 600 to 700 cP, or 700 to 1000 cP, or 700 to 900 cP, or 700 to 800 cP, or 800 to 1000 cP, or 800 to 900 cP, or 900 to 1000 cP, or about 300 cP, or about 400 cP, or about 500 cP, or about 600 cP, or about 700 cP, or about 800 cP;
1.70. Composition 1, or any of 1.1-1.69, wherein the composition has a density of 0.5 to 2.0 g/mL, e.g., about 0.6 to 1.6 g/mL, or 0.8 to 1.4 g/mL, or 0.9 to 1.3 g/mL, or 1.0 to 1.2 g/mL, or 1.05-1.15 g/mL, or about 1.1 g/mL;
1.71. Composition 1, or any of 1.1-1.70, wherein the composition is formulated for intramuscular injection;
1.72. Composition 1, or any of 1.1-1.70, wherein the composition is formulated for subcutaneous injection;
1.73. Composition 1, or any of 1.1-1.72, wherein after injection of the composition into the body (e.g., body tissue, such as muscle tissue), the composition solidifies to form a gel;
1.74. Composition 1, or any of 1.1-1.72, wherein after injection of the composition into the body (e.g., body tissue, such as muscle tissue), the solvent of the composition (e.g., N-methyl-2-pyrrolidone) is rapidly (e.g., less than 3 days) dispersed into the body tissues leaving a polymeric depot which release the lumateperone;
1.75. Composition 1.74, wherein the polymeric depot comprises from 15% to 50% by weight of lumateperone (measured as the free base equivalent) upon the completion of solvent dispersion, e.g., 20 to 50%, or 25 to 50%, or 25 to 45%, or 30 to 50%, or 30 to 45%, or about 20%, or about 25%, or about 30%, or about 35%;
1.76. Composition 1, or any of 1.1-1.75, wherein the lumateperone is in the form of a salt having low aqueous solubility, e.g., as described in any embodiment disclosed in WO 2024/030835, the contents of which are hereby incorporated by reference in its entirety;
1.77. Composition 1.76, wherein the lumateperone is in the form of a salt, e.g., a solid, crystalline salt, having an aqueous solubility of less than 2 mg/mL at pH 7, or less than 1 mg/mL at pH 7.4;
1.78. Composition 1.76 or 1.77, wherein the lumateperone is in the form of a salt, e.g., a solid, crystalline salt, selected from a 4-octylbenzenesulfonic acid addition salt, a 4-tert-butylbenzenesulfonic acid addition salt, a 4-propylbenzenesulfonic acid addition salt, a 4-ethylbenzenesulfonic acid addition salt, a 2-naphthalenesulfonic acid addition salt wherein the salt is a solvate, a solid crystalline benzenesulfonic acid addition salt characterized by a DSC thermogram lacking an endothermic event at 172-176° C., and an alkylsulfonic acid addition salt (e.g., a pentane-1-sulfonate, or a heptane-1-sulfonate);
1.79. Composition 1.76, wherein the lumateperone is in the form of a salt, e.g., a solid, crystalline salt, which is an acid addition salt with a benzenesulfonic acid substituted by one, two, or three groups R, wherein each R is independently a C₁₋₁₂alkyl group, provided that the acid is not p-toluenesulfonic acid, 4-ethylbenzenesulfonic acid, 4-propylbenzenesulfonic acid, 4-t-butylbenzenesulfonic acid, or 4-octylbenzenesulfonic acid;
1.80. Composition 1.78, wherein the lumateperone is in the form of a salt, e.g., a solid, crystalline salt, which is a 4-octylbenzenesulfonic acid addition salt (e.g., 1:1 molar ratio of lumateperone to 4-octylbenzenesulfonic acid);
1.81. Composition 1, or any of 1.1-1.80, wherein the composition comprises the lumateperone, in a net concentration of 50 to 1000 mg/mL, measured as the amount of lumateperone free base, e.g., 50 to 500 mg/mL, 50 to 400 mg/mL, 50 to 350 mg/mL, 50 to 300 mg/mL, 50 to 250 mg/mL, 50 to 200 mg/mL, 50 to 150 mg/mL, 50 to 100 mg/mL, 50 to 75 mg/mL, 75 to 100 mg/mL, 100 to 125 mg/mL, 125 to 150 mg/mL, 100 to 400 mg/mL, 100 to 350 mg/mL, 100 to 300 mg/mL, 100 to 250 mg/mL, 100 to 200 mg/mL, 100 to 150 mg/mL, 150 to 500 mg/mL, 150 to 400 mg/mL, 150 to 350 mg/mL, 150 to 300 mg/mL, 150 to 250 mg/mL, 150 to 200 mg/mL, 200 to 500 mg/mL, 200 to 400 mg/mL, 200 to 350 mg/mL, 200 to 300 mg/mL, 200 to 250 mg/mL, 250 to 500 mg/mL, 250 to 400 mg/mL, 250 to 350 mg/mL, 250 to 300 mg/mL, 300 to 500 mg/mL, 300 to 400 mg/mL, 300 to 350 mg/mL, or 400 to 500 mg/mL;
1.82. Composition 1, or any of 1.1-1.81, wherein the composition is intended to be administered once per week, once every 2 weeks, once every 3 weeks, once every 4 weeks, once every 6 weeks, once every 2 months, once every 3 months, once every 4 months, once every 5 months, or once every 6 months, e.g., once per month;
1.83. Composition 1, or any of 1.1-1.82, wherein the composition, after intramuscular or subcutaneous injection, releases the lumateperone over a period of at least 1 week, 2 weeks, 3 weeks, or 4 weeks, maintaining a plasma concentration of lumateperone of at least 5 ng/ml, e.g., at least 10 ng/ml or at least 20 ng/mL, or 5-30 ng/ml, or 10-30 ng/mL, or 5-20 ng/mL, or 10-20 ng/ml;
1.84. Composition 1, or any of 1.1-1.83, wherein the composition, after intramuscular or subcutaneous injection, releases the lumateperone over a period of at least 6 weeks maintaining a plasma concentration of lumateperone of at least 5 ng/ml, e.g., at least 10 ng/ml or at least 20 ng/mL, or 5-30 ng/ml, or 10-30 ng/mL, or 5-20 ng/mL, or 10-20 ng/ml;
1.85. Composition 1, or any of 1.1-1.84, wherein the composition, after intramuscular or subcutaneous injection, releases the lumateperone over a period of at least 8 weeks maintaining a plasma concentration of lumateperone of at least 5 ng/ml, e.g., at least 10 ng/mL or at least 20 ng/mL, or 5-30 ng/ml, or 10-30 ng/mL, or 5-20 ng/ml, or 10-20 ng/ml;
1.86. Composition 1, or any of 1.1-1.85, wherein the composition, after intramuscular or subcutaneous injection, releases the lumateperone over a period of at least 10 weeks maintaining a plasma concentration of lumateperone of at least 5 ng/ml, e.g., at least 10 ng/ml or at least 20 ng/mL, or 5-30 ng/ml, or 10-30 ng/mL, or 5-20 ng/mL, or 10-20 ng/ml;
1.87. Composition 1, or any of 1.1-1.86, wherein the composition, after intramuscular or subcutaneous injection, releases the lumateperone over a period of at least 12 weeks maintaining a plasma concentration of lumateperone of at least 5 ng/mL, e.g., at least 10 ng/ml or at least 20 ng/mL, or 5-30 ng/ml, or 10-30 ng/mL, or 5-20 ng/mL, or 10-20 ng/ml;
1.88. Composition 1, or any of 1.1-1.87, wherein the composition, after intramuscular or subcutaneous injection, releases the lumateperone at a rate to provide not more than 100 ng/ml plasma concentration of lumateperone at any time after 9 days after injection, e.g., not more than 85 ng/mL, or not more than 75 ng/ml, or not more than 70 ng/ml, or not more than 65 ng/mL, or not more than 60 ng/mL, or not more than 50 ng/ml, or not more than 40 ng/mL, or not more than 30 ng/mL, or not more than 20 ng/ml;
1.89. Composition 1, or any of 1.1-1.88, wherein the composition, after intramuscular or subcutaneous injection, releases the lumateperone at a rate to provide not more than 300 ng/mL plasma concentration of lumateperone at any time within 72 hours after injection, e.g., not more than 250 ng/mL, or not more than 200 ng/mL, or not more than 150 ng/mL, or not more than 125 ng/mL, or not more than 100 ng/ml, or not more than 75 ng/mL, or not more than 50 ng/mL, or not more than 25 ng/ml;
1.90. Composition 1, or any of 1.1-1.89, wherein the composition, after intramuscular or subcutaneous injection (e.g., of a 100 mg dose), provides a steady-state plasma concentration of lumateperone of not more than 60 ng/ml, e.g., not more than 55 ng/ml, or not more than 50 ng/mL, or not more than 45 ng/mL, or not more than 40 ng/ml;
1.91. Composition 1, or any of 1.1-1.89, wherein the composition, after intramuscular or subcutaneous injection (e.g., of a 100 mg dose), provides a steady-state plasma concentration of lumateperone of 2.5-30 ng/ml, e.g., 2.5 to 25 ng/mL, or 2.5 to 20 ng/ml, or 2.5 to 15 ng/ml or 2.5 to 10 ng/mL, or 2.5 to 5 ng/mL;
1.92. Composition 1, or any of 1.1-1.89, wherein the composition, after intramuscular or subcutaneous injection (e.g., of a 100 mg dose), provides a maximal plasma concentration (Cmax) of lumateperone of 10 to 25 ng/ml, e.g., 10-15 ng/ml, or 15 to 20 ng/mL, or 20 to 25 ng/mL, or 15 to 25 ng/ml or 10 to 20 ng/ml;
1.93. Composition 1, or any of 1.1-1.92, wherein the composition, after intramuscular or subcutaneous injection, releases not more than 10% of the total amount of lumateperone within the first 8 hours after injection, e.g., not more than 8%, or not more than 6% or not more than 5%;
1.94. Composition 1, or any of 1.1-1.93, wherein the composition comprises, or consists of, lumateperone free base or lumateperone monotosylate in an amount of 10-15% (e.g., about 12%), PLA or PLGA copolymer (e.g., PLGA 50:50, PLGA 65:35, PLGA 75:25, or PLGA 80:20) in an amount of 25-40% (e.g., about 33%), and N-methyl pyrrolidine (NMP) as the balance (e.g., 50-70%, or about 55%), optionally wherein the three components have a weight ratio of about 1:2.6:4.4;
1.95. Composition 1, or any of 1.1-1.93, wherein the composition comprises, or consists of, lumateperone free base or lumateperone monotosylate in an amount of 20-40% (e.g., about 30%), PLA or PLGA copolymer (e.g., PLGA 50:50, PLGA 65:35, PLGA 75:25, or PLGA 80:20) in an amount of 20-40% (e.g., about 30%), and N-methyl pyrrolidine (NMP) as the balance (e.g., 30-60%, or about 40%), optionally wherein the three components have a weight ratio of about 1:1:1.3;
1.96. Composition 1, or any of 1.1-1.93, wherein the composition comprises, or consists of, lumateperone free base or lumateperone monotosylate in an amount of 10-30% (e.g., about 20%), PLA or PLGA copolymer (e.g., PLGA 50:50, PLGA 65:35, PLGA 75:25, or PLGA 80:20) in an amount of 20-40% (e.g., about 30%), and N-methyl pyrrolidine (NMP) as the balance (e.g., 30-70%, or about 50%), optionally wherein the three components have a weight ratio of about 1:1.5:2.5;
1.97. Composition 1, or any of 1.1-1.93, wherein the composition comprises, or consists of, lumateperone free base or lumateperone monotosylate in an amount of 15-35% (e.g., about 25%), PLA or PLGA copolymer (e.g., PLGA 50:50, PLGA 65:35, PLGA 75:25, or PLGA 80:20) in an amount of 15-35% (e.g., about 25%), and N-methyl pyrrolidine (NMP) as the balance (e.g., 30-70%, or about 50%), optionally wherein the three components have a weight ratio of about 1:1:2;
1.98. Composition 1, or any of 1.1-1.93, wherein the composition comprises, or consists of, lumateperone free base or lumateperone monotosylate in an amount of 15-25% (e.g., about 25%), PLA or PLGA copolymer (e.g., PLGA 50:50, PLGA 65:35, PLGA 75:25, or PLGA 80:20) in an amount of 10-30% (e.g., about 17%), and N-methyl pyrrolidine (NMP) as the balance (e.g., 40-80%, or about 58%), optionally wherein the three components have a weight ratio of about 1:0.67:2.3;
1.99. Composition 1, or any of 1.1-1.93, wherein the composition comprises, or consists of, lumateperone free base or lumateperone monotosylate in an amount of 20-40% (e.g., about 30%), PLA or PLGA copolymer (e.g., PLGA 50:50, PLGA 65:35, PLGA 75:25, or PLGA 80:20) in an amount of 10-30% (e.g., about 20%), and N-methyl pyrrolidine (NMP) as the balance (e.g., 30-70%, or about 50%), optionally wherein the three components have a weight ratio of about 1:0.67:1.7;
1.100. Composition 1, or any of 1.1-1.93, wherein the composition comprises, or consists of, lumateperone free base or lumateperone monotosylate in an amount of 20-40% (e.g., about 30%), PLA or PLGA copolymer (e.g., PLGA 50:50, PLGA 65:35, PLGA 75:25, or PLGA 80:20) in an amount of 15-35% (e.g., about 27%), and N-methyl pyrrolidine (NMP) as the balance (e.g., 20-60%, or about 43%), optionally wherein the three components have a weight ratio of about 1:0.9:1.4;
1.101. Composition 1, or any of 1.1-1.93, wherein the composition comprises, or consists of, lumateperone free base or lumateperone monotosylate in an amount of 15-35% (e.g., about 25%), PLA or PLGA copolymer (e.g., PLGA 50:50, PLGA 65:35, PLGA 75:25, or PLGA 80:20) in an amount of 25-45% (e.g., about 33%), and N-methyl pyrrolidine (NMP) as the balance (e.g., 20-60%, or about 40%), optionally wherein the three components have a weight ratio of about 1:1.3:1.7;
1.102. Any of Compositions 1.94-1.101, wherein the lumateperone is lumateperone free base;
1.103. Any of Compositions 1.94-1.102, wherein the polymer is PLA (e.g., having an inherent viscosity of 0.1-0.3 dL/g, and optionally with acid end groups);
1.104. Any of Compositions 1.94-1.102, wherein the polymer is PLA (e.g., having an inherent viscosity of 0.1-0.3 dL/g, and optionally with ester end groups);
1.105. Any of Compositions 1.94-1.102, wherein the polymer is PLA (e.g., having an inherent viscosity of 0.15-0.19 dL/g, and optionally with acid end groups);
1.106. Any of Compositions 1.94-1.102, wherein the polymer is PLA (e.g., having an inherent viscosity of 0.30-0.35 dL/g, and optionally with acid end groups);
1.107. Any of Compositions 1.94-1.102, wherein the polymer is PLA (e.g., having an inherent viscosity of 0.32-0.38 dL/g, and optionally with ester end groups);
1.108. Any of Compositions 1.94-1.102, wherein the polymer is PLA (e.g., having an inherent viscosity of 0.18-0.24 dL/g, and optionally with ester end groups);
1.109. Any of Compositions 1.94-1.102, wherein the polymer is PLGA 80:20 (e.g., having an inherent viscosity of 0.2-0.4 dL/g, and optionally with acid end groups);
1.110. Any of Compositions 1.94-1.102, wherein the polymer is PLGA 80:20 (e.g., having an inherent viscosity of 0.2-0.4 dL/g, and optionally with ester end groups);
1.111. Any of Compositions 1.94-1.102, wherein the polymer is PLGA 80:20 (e.g., having an inherent viscosity of 0.25-0.29 dL/g, and optionally with acid end groups);
1.112. Any of Compositions 1.94-1.102, wherein the polymer is PLGA 80:20 (e.g., having an inherent viscosity of 0.20-0.30 dL/g, and optionally with acid end groups);
1.113. Any of Compositions 1.94-1.102, wherein the polymer is PLGA 75:25 (e.g., having an inherent viscosity of 0.15-0.25 dL/g, and optionally with acid end groups);
1.114. Any of Compositions 1.94-1.102, wherein the polymer is PLGA 75:25 (e.g., having an inherent viscosity of 0.30-0.40 dL/g, and optionally with acid end groups);
1.115. Any of Compositions 1.94-1.102, wherein the polymer is PLGA 75:25 (e.g., having an inherent viscosity of 0.35-0.45 dL/g, and optionally with acid end groups);
1.116. A single-use kit comprising a vial containing the Composition 1, or any of 1.1-1.115, and a syringe with a needle (e.g., permanently attached or detachable), with instructions for use;
1.117. A multiple-use kit comprising one or more vials containing the Composition 1, or any of 1.1-1.115, and one or more syringes with needles (e.g., permanently attached or detachable), with instructions for use;
1.118. Kit 1.116 or 1.117, wherein the needle is an 18 to 25 Gauge needle, e.g., 19 to 25 Gauge, or 20 to 25 Gauge, or 21 to 25 Gauge, or 23 to 25 Gauge;
1.119. Any one of kits 1.116-1.118, wherein the needle is a 1-1.5-inch length needle, e.g., 1.0 inch or 1.5 inch, optionally wherein the kit comprises a needle spacer between the needle and the syringe;
1.120. A pre-filled syringe (e.g., an automatic syringe) comprising Composition 1, or any of 1.1-1.115, optionally wherein the syringe needle is an 18 to 25 Gauge needle, e.g., 19 to 25 Gauge, or 20 to 25 Gauge, or 21 to 25 Gauge, or 23 to 25 Gauge;
1.121. Syringe 1.120, wherein the needle is a 1-1.5-inch length needle, e.g., 1.0 inch or 1.5 inch, optionally wherein a needle spacer is comprised between the needle and the syringe;
1.122. A kit comprising a pre-filled syringe (e.g., an automatic syringe) comprising Composition 1, or any of 1.1-1.115;
1.123. Kit 1.122, wherein the syringe needle is an 18 to 25 Gauge needle, e.g., 19 to 25 Gauge, or 20 to 25 Gauge, or 21 to 25 Gauge, or 23 to 25 Gauge;
1.124. Kit 1.122 or 1.123, wherein the needle is a 1-1.5-inch length needle, e.g., 1.0 inch or 1.5 inch, optionally wherein the kit comprises a needle spacer between the needle and the syringe;
1.125. Any of kits 1.116-1.124, wherein the syringe has a total volume of 0.3 to 2.0 mL, e.g., 0.3 to 1.5 mL, or 0.3 to 1.2 mL, or 0.3 to 1.0 mL;
1.126. A kit (e.g., single-use or multiple-use) comprising, in separate compartments, two compositions:
   (a) a first composition comprising lumateperone in free base or pharmaceutically acceptable salt form (e.g., in tosylate salt form), and, at least one of a poly(lactic acid) polymer, a poly(glycolic acid) polymer, a poly(lactic-co-glycolic acid) copolymer, and a polyester-poly(ethylene glycol) copolymer, wherein the composition does not comprise polymeric microspheres, and a second composition comprising at least one nonaqueous (e.g., organic) solvent (e.g., NMP or DMSO), optionally wherein the first composition is a dry solid, e.g., powder or granules, or an oil; or
   (b) a first composition comprising lumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, optionally wherein the second composition is a dry solid, e.g., powder or granules; or
   (c) a first composition comprising lumateperone in free base or pharmaceutically acceptable salt form (e.g., in tosylate salt form) wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), optionally wherein the first composition is a dry solid, e.g., powder or granules, or an oil; or
   (d) a first composition comprising lumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), preferably wherein the solvent of the first composition is the same as the solvent of the second composition, and optionally wherein both the first composition and the second composition are homogenous solutions;
      wherein upon combination of the first composition and the second composition, an injectable composition according to Composition 1 or any of 1.1-1.115 is formed (e.g., the lumateperone becomes dissolved or suspended in a nonaqueous liquid polymeric vehicle comprising the at least one nonaqueous (e.g., organic) solvent and the at least one of a poly(lactic acid) polymer, a poly(glycolic acid) polymer, a poly(lactic-co-glycolic acid) copolymer, and a polyester-poly(ethylene glycol) copolymer, and wherein the combination does not result in polymeric microspheres), optionally,
      wherein the injectable composition formed after combination of the first composition and the second composition is a homogenous solution;
1.127. Kit 1.126, wherein the first composition and the second composition are combined to provide an amount of lumateperone and polymer of 1000-2000 mg of the first composition (e.g., 1100-1800 mg, or 1200 to 1700 mg, or 1300-1400 mg, or 1100-1300 mg, or 1500-1700 mg, or about 1240 mg, or about 1350 mg, or about 1620 mg) and/or a volume of solvent of 1-3 mL (e.g., 1-2 mL, or 1-1.5 mL, or 1.5-2 mL, or 1-1.1 mL, or 1.1-1.5 mL, or 1.1-1.4 mL, or 1.2-1.4 mL, or about 1 mL, or about 1.3 mL, or about 1.45 mL), to form said injectable composition;
1.128. Kit 1.126 or 1.127, wherein the first composition comprises 200 to 1000 mg of lumateperone, measured as the as the amount of free base, e.g., 300 to 900 mg, or 300 to 800 mg, or 300 to 700 mg, or 300 to 600 mg, or 400 to 600 mg; and optionally wherein the second composition comprises 200 to 1000 mg of polymer or polymers, e.g., 300 to 900 mg, or 300 to 800 mg, or 300 to 700 mg, or 300 to 600 mg, or 400 to 600 mg;
1.129. Any of Kits 1.126-1.128, wherein the first composition is an oil and the second composition is a homogenous liquid (e.g., comprising or consisting of polymer and solvent, e.g., N-methyl pyrrolidone or DMSO);
1.130. Any of Kits 1.126-1.128, wherein the first composition is a dry solid, e.g. powder or granules, and the second composition is a homogenous liquid (e.g., comprising or consisting of polymer and solvent, e.g., N-methyl-2-pyrrolidone or DMSO);
1.131. Any of Kits 1.126-1.128, wherein the first composition is a homogenous liquid (e.g., comprising or consisting of lumateperone and solvent, e.g., N-methyl-2-pyrrolidone or DMSO); and the second composition is a homogenous liquid (e.g., comprising or consisting of polymer and solvent, e.g., N-methyl-2-pyrrolidone or DMSO);
1.132. Any of Kits 1.126-1.131, wherein the kit comprises the first composition in a sealed vial, the second composition in a sealed vial, and the kit further comprises a syringe and a needle;
1.133. Kit 1.132, wherein the needle is an 18 to 25 Gauge needle, e.g., 19 to 25 Gauge, or 20 to 25 Gauge, or 21 to 25 Gauge, or 23 to 25 Gauge;
1.134. Any of kits 1.126-1.133, wherein the needle is a 1-1.5-inch length needle, e.g., 1.0 inch or 1.5 inch, optionally wherein the kit comprises a needle spacer between the needle and the syringe;
1.135. Any of kits 1.126-1.134, wherein the syringe has a total volume of 0.3 to 2.0 mL, e.g., 0.3 to 1.5 mL, or 0.3 to 1.2 mL, or 0.3 to 1.0 mL;
1.136. A pre-filled dual-compartment syringe (e.g., an automatic syringe) comprising, in separate compartments, two compositions:
   (a) a first composition comprising lumateperone in free base or pharmaceutically acceptable salt form (e.g., in tosylate salt form), and, at least one of a poly(lactic acid) polymer, a poly(glycolic acid) polymer, a poly(lactic-co-glycolic acid) copolymer, and a polyester-poly(ethylene glycol) copolymer, wherein the composition does not comprise polymeric microspheres, and a second composition comprising at least one nonaqueous (e.g., organic) solvent (e.g., NMP or DMSO), optionally wherein the first composition is a dry solid, e.g., powder or granules, or an oil; or
   (b) a first composition comprising lumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, optionally wherein the second composition is a dry solid, e.g., powder or granules; or
   (c) a first composition comprising lumateperone in free base or pharmaceutically acceptable salt form (e.g., in tosylate salt form) wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), optionally wherein the first composition is a dry solid, e.g., powder or granules, or an oil; or
   (d) a first composition comprising lumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), preferably wherein the solvent of the first composition is the same as the solvent of the second composition, and optionally wherein both the first composition and the second composition are homogenous solutions;
      wherein upon actuation of the syringe the first composition and the second composition are combined to form (e.g., the lumateperone becomes dissolved or suspended in a nonaqueous liquid polymeric vehicle comprising the at least one nonaqueous (e.g., organic) solvent and the at least one of a poly(lactic acid) polymer, a poly(glycolic acid) polymer, a poly(lactic-co-glycolic acid) copolymer, and a polyester-poly(ethylene glycol) copolymer, and wherein the combination does not result in polymeric microspheres), optionally, wherein the injectable composition formed after combination of the first composition and the second composition is a homogenous solution;
1.137. Syringe 1.136, wherein the first composition and the second composition are combined to provide an amount of lumateperone and polymer of 1000-2000 mg of the first composition (e.g., 1100-1800 mg, or 1200 to 1700 mg, or 1300-1400 mg, or 1100-1300 mg, or 1500-1700 mg, or about 1240 mg, or about 1350 mg, or about 1620 mg) and/or a volume of solvent of 1-3 mL (e.g., 1-2 mL, or 1-1.5 mL, or 1.5-2 mL, or 1-1.1 mL, or 1.1-1.5 mL, or 1.1-1.4 mL, or 1.2-1.4 mL, or about 1 mL, or about 1.3 mL, or about 1.45 mL), to form said injectable composition;
1.138. Syringe 1.136 or 1.137, wherein the first composition comprises 200 to 1000 mg of lumateperone, measured as the as the amount of free base, e.g., 300 to 900 mg, or 300 to 800 mg, or 300 to 700 mg, or 300 to 600 mg, or 400 to 600 mg; and optionally wherein the second composition comprises 200 to 1000 mg of polymer or polymers, e.g., 300 to 900 mg, or 300 to 800 mg, or 300 to 700 mg, or 300 to 600 mg, or 400 to 600 mg;
1.139. Any of Syringes 1.136-1.138, wherein the first composition is an oil and the second composition is a homogenous liquid (e.g., comprising or consisting of polymer and solvent, e.g., N-methyl pyrrolidone or DMSO);
1.140. Any of Syringes 1.136-1.138, wherein the first composition is a dry solid, e.g. powder or granules, and the second composition is a homogenous liquid (e.g., comprising or consisting of polymer and solvent, e.g., N-methyl-2-pyrrolidone or DMSO);
1.141. Any of Syringes 1.136-1.138, wherein the first composition is a homogenous liquid (e.g., comprising or consisting of lumateperone and solvent, e.g., N-methyl-2-pyrrolidone or DMSO); and the second composition is a homogenous liquid (e.g., comprising or consisting of polymer and solvent, e.g., N-methyl-2-pyrrolidone);
1.142. Any of syringes 1.136-1.141, wherein the needle is a 1-1.5-inch length needle, e.g., 1.0 inch or 1.5 inch, optionally wherein the kit comprises a needle spacer between the needle and the syringe;
1.143. Any of syringes 1.136-1.142, wherein the syringe has a total volume of 0.3 to 2.0 mL, e.g., 0.3 to 1.5 mL, or 0.3 to 1.2 mL, or 0.3 to 1.0 mL;
1.144. Any preceding Composition, Kit or Syringe, wherein the administration dose of the composition, kit or syringe is 200 to 1000 mg of lumateperone, measured as the free base equivalent, delivered in a single injection, e.g., 300 to 900 mg, or 300 to 800 mg, or 300 to 700 mg, or 300 to 600 mg, or 400 to 600 mg;
1.145. Any preceding Composition, Kit or Syringe, wherein the composition is stable for at least 6 months, e.g., at least 12 months, at least 18 months, or at least 24 months, e.g., as measured by one or more of: stability of color and appearance (e.g., color and appearance are unchanged), stability of lumateperone assay (e.g., by HPLC), stability of particle size distribution, stability of viscosity, stability of water content, and stability of concentration of degradation products or impurities (e.g., wherein “stable” refers to a change from initial condition of not more than 5% of the property).

In a second aspect, the present disclosure provides a nonaqueous injectable pharmaceutical composition (Composition 2), comprising deuterolumateperone:

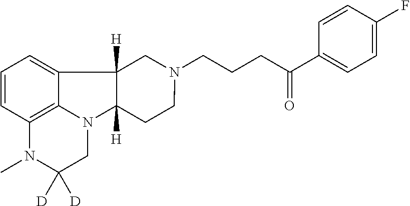 [SMILES: [2H]C1([2H])CN2c3c(cccc3N1C)[C@@H]1CN(CCCC(=O)c3ccc(F)cc3)CC[C@@H]12]

in free or pharmaceutically acceptable salt form (e.g., in free base or tosylate salt form), wherein the deuterolumateperone is dissolved or suspended in a nonaqueous liquid polymeric vehicle comprising at least one nonaqueous (e.g., organic) solvent, wherein the polymeric vehicle comprises at least one of a poly(lactic acid) polymer, a poly(glycolic acid) polymer, and a poly(lactic-co-glycolic acid) copolymer, and wherein the composition does not comprise polymeric microspheres. For example, Composition 2 may be as follows:
2.1. Composition 2, wherein the composition comprises the deuterolumateperone in free base form (e.g., in free base solid amorphous dispersion form);
2.2. Composition 2, wherein the composition comprises deuterolumateperone in pharmaceutically acceptable salt or co-crystal form;
2.3. Composition 2, wherein the composition comprises deuterolumateperone in tosylate salt form, e.g., in one or more of mono-tosylate salt form, di-tosylate salt form, and tri-tosylate salt form;
2.4. Composition 2.3, wherein the composition comprises a combination of deuterolumateperone in mono-tosylate salt form and deuterolumateperone in di-tosylate salt form;
2.5. Any of Compositions 2 or 2.1-2.3, wherein the Composition comprises deuterolumateperone in mono-tosylate salt form;
2.6. Composition 2.5, wherein the deuterolumateperone mono-tosylate is in solid crystal form, e.g., having the physical and chemical properties as disclosed in US 2023/0312573, such as one or more of the XRPD spectrum and/or DSC/TGA spectrum as disclosed therein;
2.7. Composition 2, or any of 2.1-2.6, wherein the composition comprises the deuterolumateperone (e.g., deuterolumateperone free base or deuterolumateperone tosylate) in an amount of 10 to 50% by weight of the composition, e.g., 10 to 40%, or 10 to 30%, or 10 to 25%, or 10 to 20%, or 10 to 15%, or 15 to 45%, or 15 to 40%, 15 to 35%, or 15 to 30%, or 15 to 25%, or 15 to 20%, or 20 to 45%, or 20 to 40%, or 20 to 35%, or 20 to 30%, or 20 to 25%, or 25 to 50%, or 25 to 45%, or 25 to 40%, or 25 to 35%, or 25 to 30%, or 30 to 40%, or about 15%, or about 20%, or about 25%, or about 30%, by weight of the composition, measured as the equivalent amount of free base deuterolumateperone;
2.8. Composition 2, or any of 2.1-2.7, wherein the at least one nonaqueous solvent is a pharmacologically safe water-miscible organic solvent (e.g., N-methyl-2-pyrrolidone (NMP) or DMSO) or a pharmacologically safe triglyceride oil (e.g., a vegetable oil);
2.9. Composition 2.8, wherein the organic solvent is selected from N-methyl-2-pyrrolidone (NMP), dimethylsulfoxide (DMSO), dimethyl formamide (DMF), dimethylacetamide (DMA), 2-pyrrolidone, propylene carbonate, caprolactam, N-methylcaprolactam, and triacetin;
2.10. Composition 2.9, wherein the organic solvent is N-methyl-2-pyrrolidone (NMP);
2.11. Composition 2.8, wherein the triglyceride oil is selected from peanut oil, soybean oil, sesame oil, and castor oil;
2.12. Composition 2, or any of 2.1-2.11, wherein the composition comprises any one or more nonaqueous solvents (e.g., N-methyl-2-pyrrolidone or DMSO) in a net amount of 30 to 70% by weight of the composition, e.g., 35 to 70%, or 40 to 70%, or 45 to 70%, or 50 to 70%, or 30 to 60%, or 35 to 60%, or 40 to 60%, or 45 to 60% or 50 to 60%, or 30 to 55%, or 35 to 55%, or 40 to 55%, or 45 to 55%, or 50 to 55%, by weight of the composition;
2.13. Composition 2, or any of 2.1-2.12, wherein the polymeric vehicle comprises a poly(lactic acid) polymer (e.g., poly-1-lactide, poly-d-lactide, or poly-dl-lactide);
2.14. Composition 2, or any of 2.1-2.12, wherein the polymeric vehicle comprises a poly(glycolic acid) polymer;
2.15. Composition 2, or any of 2.1-2.12, wherein the polymeric vehicle comprises a poly(lactic-co-glycolic acid) copolymer (e.g., poly(l-lactide-co-glycolide), or poly(d-lactide-co-glycolide), or poly(dl-lactide-co-glycolide);
2.16. Composition 2.15, wherein the poly(lactic-co-glycolic acid) copolymer has a lactic to glycolic molar ratio of about 99:1 to 50:50, e.g., about 99:1 to 80:20, or about 99:1 to 90:10, or about 90:10 to 75:25, or about 90:10 to 80:20, or about 80:20 to 75:25, or about 75:25 to 50:50, or about 85:15 to 75:25, or about 70:30 to 60:40;
2.17. Composition 2.15, wherein the poly(lactic-co-glycolic acid) copolymer has a lactic to glycolic molar ratio of about 84:16 to 76:24, or about 73:27 to 52:48;
2.18. Composition 2.15, wherein the poly(lactic-co-glycolic acid) copolymer has a lactic to glycolic molar ratio of about 83:17 to 77:23, or about 70:30 to 55:45;
2.19. Composition 2.15, wherein the poly(lactic-co-glycolic acid) copolymer has a lactic to glycolic molar ratio of about 82:18 to 78:22, or about 68:32 to 58:42;
2.20. Composition 2.15, wherein the poly(lactic-co-glycolic acid) copolymer has a lactic to glycolic molar ratio of about 81:19 to 79:21, or about 68:32 to 62:38;
2.21. Composition 2.15, wherein the poly(lactic-co-glycolic acid) copolymer has a lactic to glycolic molar ratio of about 75:25, or about 80:20, or about 85:15, or about 90:10, or about 95:15, or about 98:2, or about 99:1;
2.22. Composition 2.15, wherein the poly(lactic-co-glycolic acid) copolymer has a lactic to glycolic molar ratio of about 80:20;
2.23. Composition 2.15, wherein the poly(lactic-co-glycolic acid) copolymer has a lactic to glycolic molar ratio of about 65:35;
2.24. Composition 2, or any of 2.1-2.23, wherein the at least one of a poly(lactic acid) polymer, a poly(glycolic acid) polymer, and a poly(lactic-co-glycolic acid) copolymer has an inherent viscosity of 0.1 to 0.9 dL/g, e.g., 0.1 to 0.75, or 0.1 to 0.7, or 0.6 to 0.6, or 0.1 to 0.5, or 0.1 to 0.4, or 0.1 to 0.3, or 0.1 to 0.2, or 0.15 to 0.45, or 0.15 to 0.4, or 0.15 to 0.3, or 0.15 to 0.25, or 0.15 to 0.2, or 0.2 to 0.8, or 0.2 to 0.6, or 0.2 to 0.4, or 0.2 to 0.3, or 0.25 to 0.5, or 0.25 to 0.4, or 0.25 to 0.35, or 0.25 to 0.3, or 0.3 to 0.6, or 0.3 to 0.5, or 0.3 to 0.45, or 0.3 to 0.4, or 0.3 to 0.35, or 0.5 to 0.8, or 0.5 to 0.75, or 0.5 to 0.7 dL/g;
2.25. Composition 2, or any of 2.1-2.24, wherein the at least one of a poly(lactic acid) polymer, a poly(glycolic acid) polymer, and a poly(lactic-co-glycolic acid) copolymer has an average molecular weight of 5,000 to 150,000 Daltons, e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
2.26. Composition 2, or any of 2.1-2.25, wherein the at least one of a poly(lactic acid) polymer, a poly(glycolic acid) polymer, and a poly(lactic-co-glycolic acid) copolymer has a molecular weight range of 5,000 to 150,000 Daltons (e.g., at least 90% of polymer molecules have a molecular weight within the range), e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
2.27. Composition 2, or any of 2.1-2.26, wherein the at least one of a poly(lactic acid) polymer, a poly(glycolic acid) polymer, and a poly(lactic-co-glycolic acid) copolymer has carboxylic acid end groups;
2.28. Composition 2, or any of 2.1-2.26, wherein the at least one of a poly(lactic acid) polymer, a poly(glycolic acid) polymer, and a poly(lactic-co-glycolic acid) copolymer has carboxylic ester end groups (e.g., such as a C1-6 linear ester end group, e.g., methyl, ethyl, propyl, isopropyl, butyl, pentyl, or hexyl ester end groups);
2.29. Composition 2, or any of 2.1-2.28, wherein the composition comprises any one or more a poly(lactic acid) polymer, a poly(glycolic acid) polymer, and a poly(lactic-co-glycolic acid) copolymer in a net amount of 10 to 60% by weight of the composition, e.g., 10 to 50%, or 10 to 45%, or 10 to 40%, or 15 to 50%, or 15 to 45%, or 15 to 40%, or 15 to 35%, or 20 to 50%, or 20 to 45%, or 20 to 40%, or 20 to 35%, or 25 to 50%, or 25 to 45%, or 25 to 40%, or 25 to 35%, or 30 to 50%, or 30 to 45%, or 30 to 40%, or 30 to 35%, or 35 to 50%, or 35 to 45%, or 35 to 40%, or about 30%, or about 35%, or about 45%, by weight of the composition;
2.30. Composition 2, or any of 2.1-2.29, wherein the polymeric vehicle comprises a polyester-poly(ethylene glycol) copolymer, such as a poly(lactic acid)-PEG copolymer, or a poly(glycolic acid)-PEG copolymer, or a poly(lactic acid)-poly(glycolic acid)-PEG copolymer;
2.31. Composition 2.30, wherein said copolymer has a random arrangement;
2.32. Composition 2.30, wherein said copolymer has a block arrangement;
2.33. Composition 2.32, wherein said copolymer has a diblock (e.g., poly(lactic acid)-PEG, poly(glycolic acid)-PEG), or a triblock arrangement (e.g., poly(lactic acid)-PEG-poly(lactic acid), poly(glycolic acid)-PEG-poly(glycolic acid), poly(lactic acid)-PEG-poly(glycolic acid), poly(lactic acid)-poly(glycolic acid)-PEG copolymer, or poly(glycolic)-poly(lactic acid acid)-PEG copolymer;
2.34. Composition 2.33, wherein said copolymer is a poly(lactic acid)-PEG copolymer, optionally a diblock or triblock poly(lactic acid)-PEG copolymer;
2.35. Any of Compositions 2.30-2.34, wherein said copolymer has carboxylic acid end groups;
2.36. Any of Compositions 2.30-2.34, wherein said copolymer has carboxylic ester end groups (e.g., such as a C1-6 linear ester end group, e.g., methyl, ethyl, propyl, isopropyl, butyl, pentyl, or hexyl ester end groups);
2.37. Any of Compositions 2.30-2.36, wherein said copolymer is a diblock copolymer having an ether end group on the PEG block, e.g., a C1-6 linear ether end group, such as methyl, ethyl, propyl, isopropyl, butyl, pentyl, or hexyl ether end group);
2.38. Any of Compositions 2.30-2.37, wherein said copolymer has an inherent viscosity of 0.1 to 0.9 dL/g, e.g., 0.1 to 0.75, or 0.1 to 0.7, or 0.6 to 0.6, or 0.1 to 0.5, or 0.1 to 0.4, or 0.1 to 0.3, or 0.1 to 0.2, or 0.15 to 0.45, or 0.15 to 0.4, or 0.15 to 0.3, or 0.15 to 0.25, or 0.15 to 0.2, or 0.2 to 0.8, or 0.2 to 0.6, or 0.2 to 0.4, or 0.2 to 0.3, or 0.25 to 0.5, or 0.25 to 0.4, or 0.25 to 0.35, or 0.25 to 0.3, or 0.3 to 0.6, or 0.3 to 0.5, or 0.3 to 0.45, or 0.3 to 0.4, or 0.3 to 0.35, or 0.5 to 0.8, or 0.5 to 0.75, or 0.5 to 0.7 dL/g;
2.39. Any of Compositions 2.30-2.38, wherein said copolymer has an average molecular weight of 5,000 to 150,000 Daltons, e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
2.40. Any of Compositions 2.30-2.39, wherein said copolymer has a molecular weight range of 5,000 to 150,000 Daltons (e.g., at least 90% of polymer molecules have a molecular weight within the range), e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
2.41. Any of Compositions 2.30-2.40, wherein the composition comprises said copolymer in a net amount of 10 to 80% by weight of the composition, e.g., 10% to 75%, or 10% to 70%, or 10% to 65%, or 10% to 60%, or 10% to 55%, 10 to 50%, or 10 to 45%, or 10 to 40%, 15% to 75%, or 15% to 70%, or 15% to 65%, or 15% to 60%, or 15% to 55%, or 15 to 50%, or 15 to 45%, or 15 to 40%, or 15 to 35%, 20% to 75%, or 20% to 70%, or 20% to 65%, or 20% to 60%, or 20% to 55%, or 20 to 50%, or 20 to 45%, or 20 to 40%, or 20 to 35%, 25% to 75%, or 25% to 70%, or 25% to 65%, or 25% to 60%, or 25% to 55%, or 25 to 50%, or 25 to 45%, or 25 to 40%, or 25 to 35%, or 30% to 75%, or 30% to 70%, or 30% to 65%, or 30% to 60%, or 30% to 55%, or 30 to 50%, or 30 to 45%, or 30 to 40%, or 30 to 35%, or 35 to 50%, or 35 to 45%, or 35 to 40%, or about 30%, or about 35%, or about 45%, by weight of the composition;
2.42. Any of Compositions 2.30-2.41, wherein the composition comprises both a diblock polyester-poly(ethylene glycol) copolymer and a triblock polyester-poly(ethylene glycol) copolymer, each independently as described above, e.g., in a molar ratio from 6:1 to 1:6, e.g., 5:1 to 1:5, or 4:1 to 1:4, or 3:1 to 1:3, or 2:1 to 1:2, or about 4:1, or about 1:4, or about 3:1, or about 1:3, or about 2:1, or about 1:2, or about 1:1, or about 3:5, or about 5:3;
2.43. Any of Compositions 2.30-2.41, wherein the composition comprises both a diblock poly(lactic acid)-PEG copolymer and a triblock poly(lactic acid)-PEG copolymer, each independently as described above;
2.44. Composition 2.43, wherein the diblock poly(lactic acid)-PEG copolymer has a methyl ether end group (e.g., a methoxy-PEG block);
2.45. Composition 2, or any of 2.1-2.44, wherein the composition further comprises an anti-oxidant, e.g., selected from one or more of tocopherol, butylated hydroxytoluene (BHT), propyl gallate (PG, e.g., n-propyl gallate), ascorbic acid, butylated hydroxyanisole (BHA), tert-butylhydroquinone (TBHQ), carotenoids, glutathione, sodium metabisulfite, sodium ethylenediaminetetraacetate, cysteine, methionine, sesamol, citrate (e.g., sodium citrate), and citric acid, optionally in an amount of 0.1 to 1% by weight of the composition (e.g., about 0.25%, or about 0.5%, or about 0.75%);
2.46. Composition 2, or any of 2.1-2.44, wherein the composition further comprises an anti-oxidant selected from alpha-tocopherol, butylated hydroxytoluene (BHT), propyl gallate (PG, e.g., n-propyl gallate), and butylated hydroxyanisole (BHA), in an amount of 0.1 to 1% by weight of the composition (e.g., about 0.25%, or about 0.5%, or about 0.75%);
2.47. Composition 2, or any of 2.1-2.46, wherein the composition further comprises toluenesulfonic acid, e.g., in a molar ratio of about 1:1 to 1:2 with respect to the deuterolumateperone mono-tosylate, e.g., 1:1 to 1:1.5 molar ratio, or 1:1 to 1:2 molar ratio, or about a 1:1 molar ratio;
2.48. Composition 2, or any of 2.1-2.47, wherein the composition further comprises one or more additional polymers selected from polysaccharides (e.g., starches, dextrans, pectins, alginates, carrageenans, cellulose), cellulose derivatives (e.g., carboxymethyl cellulose, methylcellulose, hydroxyalkyl celluloses (e.g., hydroxyethyl cellulose, hydroxypropyl cellulose, hydroxypropyl methyl cellulose), synthetic polymers (e.g., polyvinylpyrrolidones, polyethylene oxide and/or polypropylene oxide polymers and copolymers (e.g., poloxamers, such as poloxamer 188), polyacrylate polymers (e.g., carbopols), and polyamide polymers;
2.49. Composition 2.48, wherein the composition comprises one or more of said additional polymers each in an amount of 1 to 25% by weight of the composition, e.g., from 1 to 20%, or 1 to 15%, or 1 to 10%, or 1 to 5%, or 5 to 20%, or 5 to 15%, or 5 to 10%, or 10 to 20%, or 10 to 15%, or 15 to 20%, by weight of the composition;
2.50. Composition 2, or any of 2.1-2.49, wherein the composition further comprises one or more other excipients selected from inorganic or organic acids (e.g., citric acid, lactic acid, malic acid, gluconic acid, benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid) and their salts (e.g., sodium, potassium, calcium, magnesium, lithium, ammonium salts of aforementioned acids), inorganic or organic bases (e.g., alkali metal or alkaline earth metal carbonates, bicarbonates, hydroxide, oxides), anionic surfactants (e.g., sodium lauryl sulfate, sodium laureth sulfate, sodium dodecylbenzenesulfonate, sodium lauroyl sarcosinate, sodium stearate), cationic surfactants (e.g., benzalkonium halides, cetylpyridinium halides, cetrimonium halides, benzethonium halides), zwitterionic surfactants (e.g., cocamidoalkyl betaines, such as cocamidopropyl betaine), nonionic surfactants (e.g., fatty alcohol ethoxylates (e.g., polyethylene glycol polydodecyl ethers)), sorbitan esters (e.g., sorbitan monolaurate, sorbitan monooleate, sorbitan monopalmitate, sorbitan monostearate, sorbitan tristearate), and polyethoxylated sorbitan esters (e.g., polysorbate 20, polysorbate 40, polysorbate 60, polysorbate 80);
2.51. Composition 2.50, wherein the composition comprises one or more of said additional excipients each in an amount of 0.1 to 10% by weight of the composition, e.g., from 0.1 to 8%, or 0.1 to 6%, or 0.1 to 5%, or 0.1 to 3%, or 0.1 to 2.5%, or 0.1 to 2%, or 0.1 to 1.5%, or 0.1 to 1%, or 0.1 to 0.5%, by weight of the composition;
2.52. Composition 2, or any of 2.1-2.51, wherein the polymeric vehicle consists of the one or more of a poly(lactic acid) polymer, a poly(glycolic acid) polymer, and a poly(lactic-co-glycolic acid) copolymer and the solvent (e.g., N-methyl-2-pyrrolidone or DMSO);
2.53. Composition 2, or any of 2.1-2.52, wherein the composition consists essentially of deuterolumateperone (e.g., deuterolumateperone free base), the one or more of a poly(lactic acid) polymer, a poly(glycolic acid) polymer, and a poly(lactic-co-glycolic acid) copolymer and the solvent (e.g., N-methyl-2-pyrrolidone or DMSO), and optionally an anti-oxidant (e.g., n-propyl gallate);
2.54. Composition 2, or any of 2.1-2.52, wherein the composition consists essentially of deuterolumateperone (e.g., deuterolumateperone free base), poly(lactic acid) polymer and/or poly(lactic-co-glycolic acid) copolymer, and the solvent (e.g., N-methyl-2-pyrrolidone or DMSO), and optionally an anti-oxidant (e.g., n-propyl gallate);
2.55. Composition 2, or any of 2.1-2.52, wherein the composition consists essentially of deuterolumateperone (e.g., deuterolumateperone free base), poly(lactic acid) polymer, and the solvent (e.g., N-methyl-2-pyrrolidone or DMSO), and optionally an anti-oxidant (e.g., n-propyl gallate);
2.56. Composition 2, or any of 2.1-2.52, wherein the composition consists essentially of deuterolumateperone (e.g., deuterolumateperone free base), poly(lactic-co-glycolic acid) copolymer, and the solvent (e.g., N-methyl-2-pyrrolidone or DMSO), and optionally an anti-oxidant (e.g., n-propyl gallate);
2.57. Any one of Compositions 2.52-2.56, wherein the amount of deuterolumateperone (e.g., deuterolumateperone free base) is 10-40% by weight of the composition, e.g., 15%, 20%, 25%, 30%, or 35%;
2.58. Composition 2, or any of 2.1-2.57, wherein the composition consists essentially of 10-40% deuterolumateperone (e.g., deuterolumateperone free base), 15-50% poly(lactic acid) polymer and/or poly(lactic-co-glycolic acid) copolymer (e.g., PLGA 65:35 or 80:20), and balance solvent (e.g., N-methyl-2-pyrrolidone or DMSO), and optionally an anti-oxidant (e.g., n-propyl gallate), each measured by weight of the composition;
2.59. Composition 2, or any of 2.1-2.57, wherein the composition consists essentially of 20-40% deuterolumateperone (e.g., deuterolumateperone free base), 20-30% poly(lactic acid) polymer and/or poly(lactic-co-glycolic acid) copolymer, and balance solvent (e.g., N-methyl-2-pyrrolidone or DMSO), and optionally an anti-oxidant (e.g., n-propyl gallate), each measured by weight of the composition;
2.60. Composition 2, or any of 2.1-2.57, wherein the composition consists essentially of 20-30% deuterolumateperone (e.g., deuterolumateperone free base), 25-35% poly(lactic acid) polymer and/or poly(lactic-co-glycolic acid) copolymer, and balance solvent (e.g., N-methyl-2-pyrrolidone or DMSO), and optionally an anti-oxidant (e.g., n-propyl gallate), each measured by weight of the composition;
2.61. Composition 2.60, wherein the composition comprises about 20% deuterolumateperone (e.g., deuterolumateperone free base) and 28-32% polymer (e.g., about 30% polymer), or wherein the composition comprises about 25% deuterolumateperone (e.g., deuterolumateperone free base) and 30-35% polymer (e.g., about 33% polymer), or wherein the composition comprises about 30% deuterolumateperone (e.g., deuterolumateperone free base) and 25-30% polymer (e.g., about 27% polymer);
2.62. Any of Compositions 2.52-2.61, wherein the polymer has an inherent viscosity of 0.1 to 0.5 dl/g, e.g., 0.1 to 0.4 or 0.1 to 0.3, or 0.2 to 0.5, or 0.2 to 0.4;
2.63. Composition 2, or any of 2.1-2.62, wherein the composition has no added water and comprises not more than a trace amount of water (e.g., less than 1% by weight, or less than 0.5% by weight, or less than 0.1% by weight, or less than 0.05% by weight);
2.64. Composition 2, or any of 2.1-2.63, wherein the deuterolumateperone (e.g., the deuterolumateperone free base or deuterolumateperone tosylate) is suspended in the nonaqueous liquid polymeric vehicle (e.g., to form an emulsion);
2.65. Composition 2, or any of 2.1-2.63, wherein the deuterolumateperone (e.g., the deuterolumateperone free base or deuterolumateperone tosylate) is dissolved in the nonaqueous liquid polymeric vehicle (e.g., to form a solution, such as a homogenous solution);
2.66. Composition 2, or any of 2.1-2.65, wherein the composition is a suspension or emulsion;
2.67. Composition 2, or any of 2.1-2.65, wherein the composition is a solution, e.g., a homogenous solution;
2.68. Composition 2, or any of 2.1-2.67, wherein the composition has a viscosity of 1 to 100 centiPoise (cP), e.g., 1 to 75 cP, or 1 to 70 cP, or 1 to 65 cP, or 1 to 60 cP, or 1 to 50 cP, or 1 to 40 cP, or 1 to 30 cP, or 1 to 20 cP, or 1 to 15 cP, or 1 to 10 cP, or 5 to 60 cP, or 5 to 50 cP, or 5 to 40 cP, or 5 to 30 cP, or 5 to 20 cP, or 5 to 10 cP, or 10 to 60 cP, or 10 to 50 cP, or 10 to 40 cP, or 10 to 30 cP, or 10 to 20 cP, or 20 to 60 cP, or 20 to 50 cP, or 20 to 40 cP, or 20 to 30 cP, or 30 to 60 cP, or 30 to 50 cP, or 30 to 40 cP, or 30 to 60 cP, or 40 to 50 cP, or 50 to 60 cP;
2.69. Composition 2, or any of 2.1-2.68, wherein the composition has a viscosity of 50 to 2000 centiPoise (cP), e.g., 100 to 1100 cP, or 100 to 1000 cP, or 100 to 900 cP, or 100 to 800 cP, or 100 to 700 cP, or 100 to 600 cP, or 100 to 500 cP, or 100 to 400 cP, or 1 to 300 cP, or 1 to 200 cP, or 200 to 1000 cP, or 200 to 900 cP, or 200 to 800 cP, or 200 to 700 cP, or 200 to 600 cP, or 200 to 500 cP, or 200 to 400 cP, or 200 to 300 cP, or 300 to 1000 cP, or 300 to 900 cP, or 300 to 800 cP, or 300 to 700 cP, or 300 to 600 cP, or 300 to 500 cP, or 300 to 400 cP, or 400 to 1000 cP, or 400 to 900 cP, or 400 to 800 cP, or 400 to 700 cP, or 400 to 600 cP, or 400 to 500 cP, or 500 to 1000 cP, or 500 to 900 cP, or 500 to 800 cP, or 500 to 700 cP, or 500 to 600 cP, or 600 to 1000 cP, or 600 to 900 cP, or 600 to 800 cP, or 600 to 700 cP, or 700 to 1000 cP, or 700 to 900 cP, or 700 to 800 cP, or 800 to 1000 cP, or 800 to 900 cP, or 900 to 1000 cP, or about 300 cP, or about 400 cP, or about 500 cP, or about 600 cP, or about 700 cP, or about 800 cP;
2.70. Composition 2, or any of 2.1-2.69, wherein the composition has a density of 0.5 to 2.0 g/mL, e.g., about 0.6 to 1.6 g/mL, or 0.8 to 1.4 g/mL, or 0.9 to 1.3 g/mL, or 1.0 to 1.2 g/mL, or 1.05-1.15 g/mL, or about 1.1 g/mL;
2.71. Composition 2, or any of 2.1-2.70, wherein the composition is formulated for intramuscular injection;
2.72. Composition 2, or any of 2.1-2.70, wherein the composition is formulated for subcutaneous injection;
2.73. Composition 2, or any of 2.1-2.72, wherein after injection of the composition into the body (e.g., body tissue, such as muscle tissue), the composition solidifies to form a gel;
2.74. Composition 2, or any of 2.1-2.72, wherein after injection of the composition into the body (e.g., body tissue, such as muscle tissue), the solvent of the composition (e.g., N-methyl-2-pyrrolidone) is rapidly (e.g., less than 3 days) dispersed into the body tissues leaving a polymeric depot which release the deuterolumateperone;
2.75. Composition 2.74, wherein the polymeric depot comprises from 15% to 50% by weight of deuterolumateperone (measured as the free base equivalent) upon the completion of solvent dispersion, e.g., 20 to 50%, or 25 to 50%, or 25 to 45%, or 30 to 50%, or 30 to 45%, or about 20%, or about 25%, or about 30%, or about 35%;
2.76. Composition 2, or any of 2.1-2.75, wherein the deuterolumateperone is in the form of a salt having low aqueous solubility, e.g., as described in any embodiment disclosed in WO 2024/030835, the contents of which are hereby incorporated by reference in its entirety;
2.77. Composition 2.76, wherein the deuterolumateperone is in the form of a salt, e.g., a solid, crystalline salt, having an aqueous solubility of less than 2 mg/mL at pH 7, or less than 1 mg/mL at pH 7.4;
2.78. Composition 2.76 or 2.77, wherein the deuterolumateperone is in the form of a salt, e.g., a solid, crystalline salt, selected from a 4-octylbenzenesulfonic acid addition salt, a 4-tert-butylbenzenesulfonic acid addition salt, a 4-propylbenzenesulfonic acid addition salt, a 4-ethylbenzenesulfonic acid addition salt, a 2-naphthalenesulfonic acid addition salt wherein the salt is a solvate, a solid crystalline benzenesulfonic acid addition salt characterized by a DSC thermogram lacking an endothermic event at 172-176° C., and an alkylsulfonic acid addition salt (e.g., a pentane-1-sulfonate, or a heptane-1-sulfonate);
2.79. Composition 2.76, wherein the deuterolumateperone is in the form of a salt, e.g., a solid, crystalline salt, which is an acid addition salt with a benzenesulfonic acid substituted by one, two, or three groups R, wherein each R is independently a C1-12alkyl group, provided that the acid is not p-toluenesulfonic acid, 4-ethylbenzenesulfonic acid, 4-propylbenzenesulfonic acid, 4-t-butylbenzenesulfonic acid, or 4-octylbenzenesulfonic acid;
2.80. Composition 2.78, wherein the deuterolumateperone is in the form of a salt, e.g., a solid, crystalline salt, which is a 4-octylbenzenesulfonic acid addition salt (e.g., 1:1 molar ratio of deuterolumateperone to 4-octylbenzenesulfonic acid);
2.81. Composition 2, or any of 2.1-2.80, wherein the composition comprises the deuterolumateperone, in a net concentration of 50 to 1000 mg/mL, measured as the amount of deuterolumateperone free base, e.g., 50 to 500 mg/mL, 50 to 400 mg/mL, 50 to 350 mg/mL, 50 to 300 mg/mL, 50 to 250 mg/mL, 50 to 200 mg/mL, 50 to 150 mg/mL, 50 to 100 mg/mL, 50 to 75 mg/mL, 75 to 100 mg/mL, 100 to 125 mg/mL, 125 to 150 mg/mL, 100 to 400 mg/mL, 100 to 350 mg/mL, 100 to 300 mg/mL, 100 to 250 mg/mL, 100 to 200 mg/mL, 100 to 150 mg/mL, 150 to 500 mg/mL, 150 to 400 mg/mL, 150 to 350 mg/mL, 150 to 300 mg/mL, 150 to 250 mg/mL, 150 to 200 mg/mL, 200 to 500 mg/mL, 200 to 400 mg/mL, 200 to 350 mg/mL, 200 to 300 mg/mL, 200 to 250 mg/mL, 250 to 500 mg/mL, 250 to 400 mg/mL, 250 to 350 mg/mL, 250 to 300 mg/mL, 300 to 500 mg/mL, 300 to 400 mg/mL, 300 to 350 mg/mL, or 400 to 500 mg/mL;
2.82. Composition 2, or any of 2.1-2.81, wherein the composition is intended to be administered once per week, once every 2 weeks, once every 3 weeks, once every 4 weeks, once every 6 weeks, once every 2 months, once every 3 months, once every 4 months, once every 5 months, or once every 6 months, e.g., once per month;
2.83. Composition 2, or any of 2.1-2.82, wherein the composition, after intramuscular or subcutaneous injection, releases the deuterolumateperone over a period of at least 1 week, 2 weeks, 3 weeks, or 4 weeks, maintaining a plasma concentration of deuterolumateperone of at least 5 ng/ml, e.g., at least 10 ng/ml or at least 20 ng/ml, or 5-30 ng/ml, or 10-30 ng/mL, or 5-20 ng/mL, or 10-20 ng/ml;
2.84. Composition 2, or any of 2.1-2.83, wherein the composition, after intramuscular or subcutaneous injection, releases the deuterolumateperone over a period of at least 6 weeks maintaining a plasma concentration of deuterolumateperone of at least 5 ng/ml, e.g., at least 10 ng/mL or at least 20 ng/ml, or 5-30 ng/ml, or 10-30 ng/ml, or 5-20 ng/mL, or 10-20 ng/ml;
2.85. Composition 2, or any of 2.1-2.84, wherein the composition, after intramuscular or subcutaneous injection, releases the deuterolumateperone over a period of at least 8 weeks maintaining a plasma concentration of deuterolumateperone of at least 5 ng/ml, e.g., at least 10 ng/ml or at least 20 ng/ml, or 5-30 ng/ml, or 10-30 ng/ml, or 5-20 ng/mL, or 10-20 ng/ml;
2.86. Composition 2, or any of 2.1-2.85, wherein the composition, after intramuscular or subcutaneous injection, releases the deuterolumateperone over a period of at least 10 weeks maintaining a plasma concentration of deuterolumateperone of at least 5 ng/ml, e.g., at least 10 ng/ml or at least 20 ng/mL, or 5-30 ng/ml, or 10-30 ng/ml, or 5-20 ng/mL, or 10-20 ng/ml;
2.87. Composition 2, or any of 2.1-2.86, wherein the composition, after intramuscular or subcutaneous injection, releases the deuterolumateperone over a period of at least 12 weeks maintaining a plasma concentration of deuterolumateperone of at least 5 ng/mL, e.g., at least 10 ng/ml or at least 20 ng/ml, or 5-30 ng/ml, or 10-30 ng/ml, or 5-20 ng/ml, or 10-20 ng/ml;
2.88. Composition 2, or any of 2.1-2.87, wherein the composition, after intramuscular or subcutaneous injection, releases the deuterolumateperone at a rate to provide not more than 100 ng/mL plasma concentration of deuterolumateperone at any time after 9 days after injection, e.g., not more than 85 ng/mL, or not more than 75 ng/ml, or not more than 70 ng/mL, or not more than 65 ng/ml, or not more than 60 ng/ml, or not more than 50 ng/mL, or not more than 40 ng/mL, or not more than 30 ng/ml, or not more than 20 ng/ml;
2.89. Composition 2, or any of 2.1-2.88, wherein the composition, after intramuscular or subcutaneous injection, releases the deuterolumateperone at a rate to provide not more than 300 ng/ml plasma concentration of deuterolumateperone at any time within 72 hours after injection, e.g., not more than 250 ng/ml, or not more than 200 ng/ml, or not more than 150 ng/mL, or not more than 125 ng/mL, or not more than 100 ng/ml, or not more than 75 ng/mL, or not more than 50 ng/mL, or not more than 25 ng/mL;
2.90. Composition 2, or any of 2.1-2.89, wherein the composition, after intramuscular or subcutaneous injection (e.g., of a 100 mg dose), provides a steady-state plasma concentration of deuterolumateperone of not more than 60 ng/ml, e.g., not more than 55 ng/ml, or not more than 50 ng/mL, or not more than 45 ng/ml, or not more than 40 ng/ml;
2.91. Composition 2, or any of 2.1-2.89, wherein the composition, after intramuscular or subcutaneous injection (e.g., of a 100 mg dose), provides a steady-state plasma concentration of deuterolumateperone of 2.5-30 ng/ml, e.g., 2.5 to 25 ng/mL, or 2.5 to 20 ng/ml, or 2.5 to 15 ng/mL or 2.5 to 10 ng/ml, or 2.5 to 5 ng/ml;
2.92. Composition 2, or any of 2.1-2.89, wherein the composition, after intramuscular or subcutaneous injection (e.g., of a 100 mg dose), provides a maximal plasma concentration (Cmax) of deuterolumateperone of 10 to 25 ng/ml, e.g., 10-15 ng/mL, or 15 to 20 ng/ml, or 20 to 25 ng/mL, or 15 to 25 ng/ml or 10 to 20 ng/ml;
2.93. Composition 2, or any of 2.1-2.92, wherein the composition, after intramuscular or subcutaneous injection, releases not more than 10% of the total amount of deuterolumateperone within the first 8 hours after injection, e.g., not more than 8%, or not more than 6% or not more than 5%;
2.94. Composition 2, or any of 2.1-2.93, wherein the composition comprises, or consists of, deuterolumateperone free base or deuterolumateperone monotosylate in an amount of 10-15% (e.g., about 12%), PLA or PLGA copolymer (e.g., PLGA 50:50, PLGA 65:35, PLGA 75:25, or PLGA 80:20) in an amount of 25-40% (e.g., about 33%), and N-methyl pyrrolidine (NMP) as the balance (e.g., 50-70%, or about 55%), optionally wherein the three components have a weight ratio of about 1:2.6:4.4;
2.95. Composition 2, or any of 2.1-2.93, wherein the composition comprises, or consists of, deuterolumateperone free base or deuterolumateperone monotosylate in an amount of 20-40% (e.g., about 30%), PLA or PLGA copolymer (e.g., PLGA 50:50, PLGA 65:35, PLGA 75:25, or PLGA 80:20) in an amount of 20-40% (e.g., about 30%), and N-methyl pyrrolidine (NMP) as the balance (e.g., 30-60%, or about 40%), optionally wherein the three components have a weight ratio of about 1:1:1.3;
2.96. Composition 2, or any of 2.1-2.93, wherein the composition comprises, or consists of, deuterolumateperone free base or deuterolumateperone monotosylate in an amount of 10-30% (e.g., about 20%), PLA or PLGA copolymer (e.g., PLGA 50:50, PLGA 65:35, PLGA 75:25, or PLGA 80:20) in an amount of 20-40% (e.g., about 30%), and N-methyl pyrrolidine (NMP) as the balance (e.g., 30-70%, or about 50%), optionally wherein the three components have a weight ratio of about 1:1.5:2.5;
2.97. Composition 2, or any of 2.1-2.93, wherein the composition comprises, or consists of, deuterolumateperone free base or deuterolumateperone monotosylate in an amount of 15-35% (e.g., about 25%), PLA or PLGA copolymer (e.g., PLGA 50:50, PLGA 65:35, PLGA 75:25, or PLGA 80:20) in an amount of 15-35% (e.g., about 25%), and N-methyl pyrrolidine (NMP) as the balance (e.g., 30-70%, or about 50%), optionally wherein the three components have a weight ratio of about 1:1:2;
2.98. Composition 2, or any of 2.1-2.93, wherein the composition comprises, or consists of, deuterolumateperone free base or deuterolumateperone monotosylate in an amount of 15-25% (e.g., about 25%), PLA or PLGA copolymer (e.g., PLGA 50:50, PLGA 65:35, PLGA 75:25, or PLGA 80:20) in an amount of 10-30% (e.g., about 17%), and N-methyl pyrrolidine (NMP) as the balance (e.g., 40-80%, or about 58%), optionally wherein the three components have a weight ratio of about 1:0.67:2.3;
2.99. Composition 2, or any of 2.1-2.93, wherein the composition comprises, or consists of, deuterolumateperone free base or deuterolumateperone monotosylate in an amount of 20-40% (e.g., about 30%), PLA or PLGA copolymer (e.g., PLGA 50:50, PLGA 65:35, PLGA 75:25, or PLGA 80:20) in an amount of 10-30% (e.g., about 20%), and N-methyl pyrrolidine (NMP) as the balance (e.g., 30-70%, or about 50%), optionally wherein the three components have a weight ratio of about 1:0.67:1.7;
2.100. Composition 2, or any of 2.1-2.93, wherein the composition comprises, or consists of, deuterolumateperone free base or deuterolumateperone monotosylate in an amount of 20-40% (e.g., about 30%), PLA or PLGA copolymer (e.g., PLGA 50:50, PLGA 65:35, PLGA 75:25, or PLGA 80:20) in an amount of 15-35% (e.g., about 27%), and N-methyl pyrrolidine (NMP) as the balance (e.g., 20-60%, or about 43%), optionally wherein the three components have a weight ratio of about 1:0.9:1.4;
2.101. Composition 2, or any of 2.1-2.93, wherein the composition comprises, or consists of, deuterolumateperone free base or deuterolumateperone monotosylate in an amount of 15-35% (e.g., about 25%), PLA or PLGA copolymer (e.g., PLGA 50:50, PLGA 65:35, PLGA 75:25, or PLGA 80:20) in an amount of 25-45% (e.g., about 33%), and N-methyl pyrrolidine (NMP) as the balance (e.g., 20-60%, or about 40%), optionally wherein the three components have a weight ratio of about 1:1.3:1.7;
2.102. Any of Compositions 2.94-2.101, wherein the deuterolumateperone is deuterolumateperone free base;
2.103. Any of Compositions 2.94-2.102, wherein the polymer is PLA (e.g., having an inherent viscosity of 0.1-0.3 dL/g, and optionally with acid end groups);
2.104. Any of Compositions 2.94-2.102, wherein the polymer is PLA (e.g., having an inherent viscosity of 0.1-0.3 dL/g, and optionally with ester end groups);
2.105. Any of Compositions 2.94-2.102, wherein the polymer is PLA (e.g., having an inherent viscosity of 0.15-0.19 dL/g, and optionally with acid end groups);
2.106. Any of Compositions 2.94-2.102, wherein the polymer is PLA (e.g., having an inherent viscosity of 0.30-0.35 dL/g, and optionally with acid end groups);
2.107. Any of Compositions 2.94-2.102, wherein the polymer is PLA (e.g., having an inherent viscosity of 0.32-0.38 dL/g, and optionally with ester end groups);
2.108. Any of Compositions 2.94-2.102, wherein the polymer is PLA (e.g., having an inherent viscosity of 0.18-0.24 dL/g, and optionally with ester end groups);
2.109. Any of Compositions 2.94-2.102, wherein the polymer is PLGA 80:20 (e.g., having an inherent viscosity of 0.2-0.4 dL/g, and optionally with acid end groups);
2.110. Any of Compositions 2.94-2.102, wherein the polymer is PLGA 80:20 (e.g., having an inherent viscosity of 0.2-0.4 dL/g, and optionally with ester end groups);
2.111. Any of Compositions 2.94-2.102, wherein the polymer is PLGA 80:20 (e.g., having an inherent viscosity of 0.25-0.29 dL/g, and optionally with acid end groups);
2.112. Any of Compositions 2.94-2.102, wherein the polymer is PLGA 80:20 (e.g., having an inherent viscosity of 0.20-0.30 dL/g, and optionally with acid end groups);
2.113. Any of Compositions 2.94-2.102, wherein the polymer is PLGA 75:25 (e.g., having an inherent viscosity of 0.15-0.25 dL/g, and optionally with acid end groups);
2.114. Any of Compositions 2.94-2.102, wherein the polymer is PLGA 75:25 (e.g., having an inherent viscosity of 0.30-0.40 dL/g, and optionally with acid end groups);
2.115. Any of Compositions 2.94-2.102, wherein the polymer is PLGA 75:25 (e.g., having an inherent viscosity of 0.35-0.45 dL/g, and optionally with acid end groups);
2.116. A single-use kit comprising a vial containing the Composition 2, or any of 2.1-2.115, and a syringe with a needle (e.g., permanently attached or detachable), with instructions for use;
2.117. A multiple-use kit comprising one or more vials containing the Composition 2, or any of 2.1-2.115, and one or more syringes with needles (e.g., permanently attached or detachable), with instructions for use;
2.118. Kit 2.116 or 2.117, wherein the needle is an 18 to 25 Gauge needle, e.g., 19 to 25 Gauge, or 20 to 25 Gauge, or 21 to 25 Gauge, or 23 to 25 Gauge;
2.119. Any one of kits 2.116-2.118, wherein the needle is a 1-1.5-inch length needle, e.g., 1.0 inch or 1.5 inch, optionally wherein the kit comprises a needle spacer between the needle and the syringe;
2.120. A pre-filled syringe (e.g., an automatic syringe) comprising Composition 2, or any of 2.1-2.115, optionally wherein the syringe needle is an 18 to 25 Gauge needle, e.g., 19 to 25 Gauge, or 20 to 25 Gauge, or 21 to 25 Gauge, or 23 to 25 Gauge;
2.121. Syringe 2.120, wherein the needle is a 1-1.5-inch length needle, e.g., 1.0 inch or 1.5 inch, optionally wherein a needle spacer is comprised between the needle and the syringe;
2.122. A kit comprising a pre-filled syringe (e.g., an automatic syringe) comprising Composition 2, or any of 2.1-2.115;
2.123. Kit 2.122, wherein the syringe needle is an 18 to 25 Gauge needle, e.g., 19 to 25 Gauge, or 20 to 25 Gauge, or 21 to 25 Gauge, or 23 to 25 Gauge;
2.124. Kit 2.122 or 2.123, wherein the needle is a 1-1.5-inch length needle, e.g., 1.0 inch or 1.5 inch, optionally wherein the kit comprises a needle spacer between the needle and the syringe;
2.125. Any of kits 2.116-2.124, wherein the syringe has a total volume of 0.3 to 2.0 mL, e.g., 0.3 to 1.5 mL, or 0.3 to 1.2 mL, or 0.3 to 1.0 mL;
2.126. A kit (e.g., single-use or multiple-use) comprising, in separate compartments, two compositions:
   (a) a first composition comprising deuterolumateperone in free base or pharmaceutically acceptable salt form (e.g., in tosylate salt form), and, at least one of a poly(lactic acid) polymer, a poly(glycolic acid) polymer, a poly(lactic-co-glycolic acid) copolymer, and a polyester-poly(ethylene glycol) copolymer, wherein the composition does not comprise polymeric microspheres, and a second composition comprising at least one nonaqueous (e.g., organic) solvent (e.g., NMP or DMSO), optionally wherein the first composition is a dry solid, e.g., powder or granules, or an oil; or
   (b) a first composition comprising deuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, optionally wherein the second composition is a dry solid, e.g., powder or granules; or
   (c) a first composition comprising deuterolumateperone in free base or pharmaceutically acceptable salt form (e.g., in tosylate salt form) wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), optionally wherein the first composition is a dry solid, e.g., powder or granules, or an oil; or
   (d) a first composition comprising deuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), preferably wherein the solvent of the first composition is the same as the solvent of the second composition, and optionally wherein both the first composition and the second composition are homogenous solutions;
      wherein upon combination of the first composition and the second composition, an injectable composition according to Composition 2 or any of 2.1-2.115 is formed (e.g., the deuterolumateperone becomes dissolved or suspended in a nonaqueous liquid polymeric vehicle comprising the at least one nonaqueous (e.g., organic) solvent and the at least one of a poly(lactic acid) polymer, a poly(glycolic acid) polymer, a poly(lactic-co-glycolic acid) copolymer, and a polyester-poly(ethylene glycol) copolymer, and wherein the combination does not result in polymeric microspheres), optionally,
      wherein the injectable composition formed after combination of the first composition and the second composition is a homogenous solution;
2.127. Kit 2.126, wherein the first composition and the second composition are combined to provide an amount of deuterolumateperone and polymer of 1000-2000 mg of the first composition (e.g., 1100-1800 mg, or 1200 to 1700 mg, or 1300-1400 mg, or 1100-1300 mg, or 1500-1700 mg, or about 1240 mg, or about 1350 mg, or about 1620 mg) and/or a volume of solvent of 1-3 mL (e.g., 1-2 mL, or 1-1.5 mL, or 1.5-2 mL, or 1-1.1 mL, or 1.1-1.5 mL, or 1.1-1.4 mL, or 1.2-1.4 mL, or about 1 mL, or about 1.3 mL, or about 1.45 mL), to form said injectable composition;
2.128. Kit 2.126 or 2.127, wherein the first composition comprises 200 to 1000 mg of deuterolumateperone, measured as the as the amount of free base, e.g., 300 to 900 mg, or 300 to 800 mg, or 300 to 700 mg, or 300 to 600 mg, or 400 to 600 mg; and optionally wherein the second composition comprises 200 to 1000 mg of polymer or polymers, e.g., 300 to 900 mg, or 300 to 800 mg, or 300 to 700 mg, or 300 to 600 mg, or 400 to 600 mg;
2.129. Any of Kits 2.126-2.128, wherein the first composition is an oil and the second composition is a homogenous liquid (e.g., comprising or consisting of polymer and solvent, e.g., N-methyl pyrrolidone or DMSO);
2.130. Any of Kits 2.126-2.128, wherein the first composition is a dry solid, e.g. powder or granules, and the second composition is a homogenous liquid (e.g., comprising or consisting of polymer and solvent, e.g., N-methyl-2-pyrrolidone or DMSO);
2.131. Any of Kits 2.126-2.131, wherein the first composition is a homogenous liquid (e.g., comprising or consisting of deuterolumateperone and solvent, e.g., N-methyl-2-pyrrolidone); and the second composition is a homogenous liquid (e.g., comprising or consisting of polymer and solvent, e.g., N-methyl-2-pyrrolidone or DMSO);
2.132. Any of Kits 2.126-2.131, wherein the kit comprises the first composition in a sealed vial, the second composition in a sealed vial, and the kit further comprises a syringe and a needle;
2.133. Kit 2.132, wherein the needle is an 18 to 25 Gauge needle, e.g., 19 to 25 Gauge, or 20 to 25 Gauge, or 21 to 25 Gauge, or 23 to 25 Gauge;
2.134. Any of kits 2.126-2.133, wherein the needle is a 1-1.5-inch length needle, e.g., 1.0 inch or 1.5 inch, optionally wherein the kit comprises a needle spacer between the needle and the syringe;
2.135. Any of kits 2.126-2.134, wherein the syringe has a total volume of 0.3 to 2.0 mL, e.g., 0.3 to 1.5 mL, or 0.3 to 1.2 mL, or 0.3 to 1.0 mL;
2.136. A pre-filled dual-compartment syringe (e.g., an automatic syringe) comprising, in separate compartments, two compositions:
   (a) a first composition comprising deuterolumateperone in free base or pharmaceutically acceptable salt form (e.g., in tosylate salt form), and, at least one of a poly(lactic acid) polymer, a poly(glycolic acid) polymer, a poly(lactic-co-glycolic acid) copolymer, and a polyester-poly(ethylene glycol) copolymer, wherein the composition does not comprise polymeric microspheres, and a second composition comprising at least one nonaqueous (e.g., organic) solvent (e.g., NMP or DMSO), optionally wherein the first composition is a dry solid, e.g., powder or granules, or an oil; or
   (b) a first composition comprising deuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, optionally wherein the second composition is a dry solid, e.g., powder or granules; or
   (c) a first composition comprising deuterolumateperone in free base or pharmaceutically acceptable salt form (e.g., in tosylate salt form) wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), optionally wherein the first composition is a dry solid, e.g., powder or granules, or an oil; or
   (d) a first composition comprising deuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), preferably wherein the solvent of the first composition is the same as the solvent of the second composition, and optionally wherein both the first composition and the second composition are homogenous solutions;
      wherein upon actuation of the syringe the first composition and the second composition are combined to form (e.g., the deuterolumateperone becomes dissolved or suspended in a nonaqueous liquid polymeric vehicle comprising the at least one nonaqueous (e.g., organic) solvent and the at least one of a poly(lactic acid) polymer, a poly(glycolic acid) polymer, a poly(lactic-co-glycolic acid) copolymer, and a polyester-poly(ethylene glycol) copolymer, and wherein the combination does not result in polymeric microspheres), optionally,
      wherein the injectable composition formed after combination of the first composition and the second composition is a homogenous solution;
2.137. Syringe 2.136, wherein the first composition and the second composition are combined to provide an amount of deuterolumateperone and polymer of 1000-2000 mg of the first composition (e.g., 1100-1800 mg, or 1200 to 1700 mg, or 1300-1400 mg, or 1100-1300 mg, or 1500-1700 mg, or about 1240 mg, or about 1350 mg, or about 1620 mg) and/or a volume of solvent of 1-3 mL (e.g., 1-2 mL, or 1-1.5 mL, or 1.5-2 mL, or 1-1.1 mL, or 1.1-1.5 mL, or 1.1-1.4 mL, or 1.2-1.4 mL, or about 1 mL, or about 1.3 mL, or about 1.45 mL), to form said injectable composition;
2.138. Syringe 2.136 or 2.137, wherein the first composition comprises 200 to 1000 mg of deuterolumateperone, measured as the as the amount of free base, e.g., 300 to 900 mg, or 300 to 800 mg, or 300 to 700 mg, or 300 to 600 mg, or 400 to 600 mg; and optionally wherein the second composition comprises 200 to 1000 mg of polymer or polymers, e.g., 300 to 900 mg, or 300 to 800 mg, or 300 to 700 mg, or 300 to 600 mg, or 400 to 600 mg;
2.139. Any of Syringes 2.136-2.138, wherein the first composition is an oil and the second composition is a homogenous liquid (e.g., comprising or consisting of polymer and solvent, e.g., N-methyl pyrrolidone or DMSO);
2.140. Any of Syringes 2.136-2.138, wherein the first composition is a dry solid, e.g. powder or granules, and the second composition is a homogenous liquid (e.g., comprising or consisting of polymer and solvent, e.g., N-methyl-2-pyrrolidone or DMSO);
2.141. Any of Syringes 2.136-2.138, wherein the first composition is a homogenous liquid (e.g., comprising or consisting of deuterolumateperone and solvent, e.g., N-methyl-2-pyrrolidone); and the second composition is a homogenous liquid (e.g., comprising or consisting of polymer and solvent, e.g., N-methyl-2-pyrrolidone or DMSO);
2.142. Any of syringes 2.136-2.141, wherein the needle is a 1-1.5-inch length needle, e.g., 1.0 inch or 1.5 inch, optionally wherein the kit comprises a needle spacer between the needle and the syringe;
2.143. Any of syringes 2.136-2.142, wherein the syringe has a total volume of 0.3 to 2.0 mL, e.g., 0.3 to 1.5 mL, or 0.3 to 1.2 mL, or 0.3 to 1.0 mL;
2.144. Any preceding Composition, Kit or Syringe, wherein the administration dose of the composition, kit or syringe is 200 to 1000 mg of deuterolumateperone, measured as the free base equivalent, delivered in a single injection, e.g., 300 to 900 mg, or 300 to 800 mg, or 300 to 700 mg, or 300 to 600 mg, or 400 to 600 mg;
2.145. Any preceding Composition, Kit or Syringe, wherein the composition is stable for at least 6 months, e.g., at least 12 months, at least 18 months, or at least 24 months, e.g., as measured by one or more of: stability of color and appearance (e.g., color and appearance are unchanged), stability of deuterolumateperone assay (e.g., by HPLC), stability of particle size distribution, stability of viscosity, stability of water content, and stability of concentration of degradation products or impurities (e.g., wherein “stable” refers to a change from initial condition of not more than 5% of the property).

In a third aspect, the present disclosure provides a nonaqueous injectable pharmaceutical composition (Composition 3), comprising tetradeuterolumateperone:

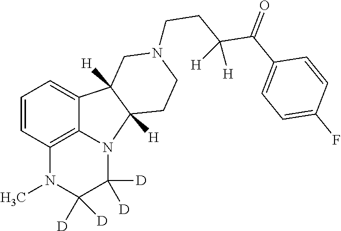 [SMILES: [2H]C1([2H])N(C)c2cccc3c2N([C@H]2CCN(CCCC(=O)c4ccc(F)cc4)C[C@@H]32)C1([2H])[2H]]

in free or pharmaceutically acceptable salt form (e.g., in free base or tosylate salt form), wherein the tetradeuterolumateperone is dissolved or suspended in a nonaqueous liquid polymeric vehicle comprising at least one nonaqueous (e.g., organic) solvent, wherein the polymeric vehicle comprises at least one of a poly(lactic acid) polymer, a poly(glycolic acid) polymer, and a poly(lactic-co-glycolic acid) copolymer, and wherein the composition does not comprise polymeric microspheres. For example, Composition 3 may be as follows:
3.1. Composition 3, wherein the composition comprises the tetradeuterolumateperone in free base form (e.g., in free base solid amorphous dispersion form);
3.2. Composition 3, wherein the composition comprises tetradeuterolumateperone in pharmaceutically acceptable salt or co-crystal form;
3.3. Composition 3, wherein the composition comprises tetradeuterolumateperone in tosylate salt form, e.g., in one or more of mono-tosylate salt form, di-tosylate salt form, and tri-tosylate salt form;
3.4. Composition 3.3, wherein the composition comprises a combination of tetradeuterolumateperone in mono-tosylate salt form and tetradeuterolumateperone in di-tosylate salt form;
3.5. Any of Compositions 3 or 3.1-3.3, wherein the Composition comprises tetradeuterolumateperone in mono-tosylate salt form;
3.6. Composition 3.5, wherein the tetradeuterolumateperone mono-tosylate is in solid form, e.g., solid crystal form;
3.7. Composition 3, or any of 3.1-3.6, wherein the composition comprises the tetradeuterolumateperone (e.g., tetradeuterolumateperone free base or tetradeuterolumateperone tosylate) in an amount of 10 to 50% by weight of the composition, e.g., 10 to 40%, or 10 to 30%, or 10 to 25%, or 10 to 20%, or 10 to 15%, or 15 to 45%, or 15 to 40%, 15 to 35%, or 15 to 30%, or 15 to 25%, or 15 to 20%, or 20 to 45%, or 20 to 40%, or 20 to 35%, or 20 to 30%, or 20 to 25%, or 25 to 50%, or 25 to 45%, or 25 to 40%, or 25 to 35%, or 25 to 30%, or 30 to 40%, or about 15%, or about 20%, or about 25%, or about 30%, by weight of the composition, measured as the equivalent amount of free base tetradeuterolumateperone;
3.8. Composition 3, or any of 3.1-3.7, wherein the at least one nonaqueous solvent is a pharmacologically safe water-miscible organic solvent (e.g., N-methyl-2-pyrrolidone (NMP) or DMSO) or a pharmacologically safe triglyceride oil (e.g., a vegetable oil);
3.9. Composition 3.8, wherein the organic solvent is selected from N-methyl-2-pyrrolidone (NMP), dimethylsulfoxide (DMSO), dimethyl formamide (DMF), dimethylacetamide (DMA), 2-pyrrolidone, propylene carbonate, caprolactam, N-methylcaprolactam, and triacetin;
3.10. Composition 3.9, wherein the organic solvent is N-methyl-2-pyrrolidone (NMP);
3.11. Composition 3.8, wherein the triglyceride oil is selected from peanut oil, soybean oil, sesame oil, and castor oil;
3.12. Composition 3, or any of 3.1-3.11, wherein the composition comprises any one or more nonaqueous solvents (e.g., N-methyl-2-pyrrolidone or DMSO) in a net amount of 30 to 70% by weight of the composition, e.g., 35 to 70%, or 40 to 70%, or 45 to 70%, or 50 to 70%, or 30 to 60%, or 35 to 60%, or 40 to 60%, or 45 to 60% or 50 to 60%, or 30 to 55%, or 35 to 55%, or 40 to 55%, or 45 to 55%, or 50 to 55%, by weight of the composition;
3.13. Composition 3, or any of 3.1-3.12, wherein the polymeric vehicle comprises a poly(lactic acid) polymer (e.g., poly-1-lactide, poly-d-lactide, or poly-dl-lactide);
3.14. Composition 3, or any of 3.1-3.12, wherein the polymeric vehicle comprises a poly(glycolic acid) polymer;
3.15. Composition 3, or any of 3.1-3.12, wherein the polymeric vehicle comprises a poly(lactic-co-glycolic acid) copolymer (e.g., poly(l-lactide-co-glycolide), or poly(d-lactide-co-glycolide), or poly(dl-lactide-co-glycolide);
3.16. Composition 3.15, wherein the poly(lactic-co-glycolic acid) copolymer has a lactic to glycolic molar ratio of about 99:1 to 50:50, e.g., about 99:1 to 80:20, or about 99:1 to 90:10, or about 90:10 to 75:25, or about 90:10 to 80:20, or about 80:20 to 75:25, or about 75:25 to 50:50, or about 85:15 to 75:25, or about 70:30 to 60:40;
3.17. Composition 3.15, wherein the poly(lactic-co-glycolic acid) copolymer has a lactic to glycolic molar ratio of about 84:16 to 76:24, or about 73:27 to 52:48;
3.18. Composition 3.15, wherein the poly(lactic-co-glycolic acid) copolymer has a lactic to glycolic molar ratio of about 83:17 to 77:23, or about 70:30 to 55:45;
3.19. Composition 3.15, wherein the poly(lactic-co-glycolic acid) copolymer has a lactic to glycolic molar ratio of about 82:18 to 78:22, or about 68:32 to 58:42;
3.20. Composition 3.15, wherein the poly(lactic-co-glycolic acid) copolymer has a lactic to glycolic molar ratio of about 81:19 to 79:21, or about 68:32 to 62:38;
3.21. Composition 3.15, wherein the poly(lactic-co-glycolic acid) copolymer has a lactic to glycolic molar ratio of about 75:25, or about 80:20, or about 85:15, or about 90:10, or about 95:15, or about 98:2, or about 99:1;
3.22. Composition 3.15, wherein the poly(lactic-co-glycolic acid) copolymer has a lactic to glycolic molar ratio of about 80:20;
3.23. Composition 3.15, wherein the poly(lactic-co-glycolic acid) copolymer has a lactic to glycolic molar ratio of about 65:35;
3.24. Composition 3, or any of 3.1-3.23, wherein the at least one of a poly(lactic acid) polymer, a poly(glycolic acid) polymer, and a poly(lactic-co-glycolic acid) copolymer has an inherent viscosity of 0.1 to 0.9 dL/g, e.g., 0.1 to 0.75, or 0.1 to 0.7, or 0.6 to 0.6, or 0.1 to 0.5, or 0.1 to 0.4, or 0.1 to 0.3, or 0.1 to 0.2, or 0.15 to 0.45, or 0.15 to 0.4, or 0.15 to 0.3, or 0.15 to 0.25, or 0.15 to 0.2, or 0.2 to 0.8, or 0.2 to 0.6, or 0.2 to 0.4, or 0.2 to 0.3, or 0.25 to 0.5, or 0.25 to 0.4, or 0.25 to 0.35, or 0.25 to 0.3, or 0.3 to 0.6, or 0.3 to 0.5, or 0.3 to 0.45, or 0.3 to 0.4, or 0.3 to 0.35, or 0.5 to 0.8, or 0.5 to 0.75, or 0.5 to 0.7 dL/g;
3.25. Composition 3, or any of 3.1-3.24, wherein the at least one of a poly(lactic acid) polymer, a poly(glycolic acid) polymer, and a poly(lactic-co-glycolic acid) copolymer has an average molecular weight of 5,000 to 150,000 Daltons, e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
3.26. Composition 3, or any of 3.1-3.25, wherein the at least one of a poly(lactic acid) polymer, a poly(glycolic acid) polymer, and a poly(lactic-co-glycolic acid) copolymer has a molecular weight range of 5,000 to 150,000 Daltons (e.g., at least 90% of polymer molecules have a molecular weight within the range), e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
3.27. Composition 3, or any of 3.1-3.26, wherein the at least one of a poly(lactic acid) polymer, a poly(glycolic acid) polymer, and a poly(lactic-co-glycolic acid) copolymer has carboxylic acid end groups;
3.28. Composition 3, or any of 3.1-3.26, wherein the at least one of a poly(lactic acid) polymer, a poly(glycolic acid) polymer, and a poly(lactic-co-glycolic acid) copolymer has carboxylic ester end groups (e.g., such as a C1-6 linear ester end group, e.g., methyl, ethyl, propyl, isopropyl, butyl, pentyl, or hexyl ester end groups);
3.29. Composition 3, or any of 3.1-3.28, wherein the composition comprises any one or more a poly(lactic acid) polymer, a poly(glycolic acid) polymer, and a poly(lactic-co-glycolic acid) copolymer in a net amount of 10 to 60% by weight of the composition, e.g., 10 to 50%, or 10 to 45%, or 10 to 40%, or 15 to 50%, or 15 to 45%, or 15 to 40%, or 15 to 35%, or 20 to 50%, or 20 to 45%, or 20 to 40%, or 20 to 35%, or 25 to 50%, or 25 to 45%, or 25 to 40%, or 25 to 35%, or 30 to 50%, or 30 to 45%, or 30 to 40%, or 30 to 35%, or 35 to 50%, or 35 to 45%, or 35 to 40%, or about 30%, or about 35%, or about 45%, by weight of the composition;
3.30. Composition 3, or any of 3.1-3.29, wherein the polymeric vehicle comprises a polyester-poly(ethylene glycol) copolymer, such as a poly(lactic acid)-PEG copolymer, or a poly(glycolic acid)-PEG copolymer, or a poly(lactic acid)-poly(glycolic acid)-PEG copolymer;
3.31. Composition 3.30, wherein said copolymer has a random arrangement;
3.32. Composition 3.30, wherein said copolymer has a block arrangement;
3.33. Composition 3.32, wherein said copolymer has a diblock (e.g., poly(lactic acid)-PEG, poly(glycolic acid)-PEG), or a triblock arrangement (e.g., poly(lactic acid)-PEG-poly(lactic acid), poly(glycolic acid)-PEG-poly(glycolic acid), poly(lactic acid)-PEG-poly(glycolic acid), poly(lactic acid)-poly(glycolic acid)-PEG copolymer, or poly(glycolic)-poly(lactic acid acid)-PEG copolymer;
3.34. Composition 3.33, wherein said copolymer is a poly(lactic acid)-PEG copolymer, optionally a diblock or triblock poly(lactic acid)-PEG copolymer;
3.35. Any of Compositions 3.30-3.34, wherein said copolymer has carboxylic acid end groups;
3.36. Any of Compositions 3.30-3.34, wherein said copolymer has carboxylic ester end groups (e.g., such as a C1-6 linear ester end group, e.g., methyl, ethyl, propyl, isopropyl, butyl, pentyl, or hexyl ester end groups);
3.37. Any of Compositions 3.30-3.36, wherein said copolymer is a diblock copolymer having an ether end group on the PEG block, e.g., a C1-6 linear ether end group, such as methyl, ethyl, propyl, isopropyl, butyl, pentyl, or hexyl ether end group);
3.38. Any of Compositions 3.30-3.37, wherein said copolymer has an inherent viscosity of 0.1 to 0.9 dL/g, e.g., 0.1 to 0.75, or 0.1 to 0.7, or 0.6 to 0.6, or 0.1 to 0.5, or 0.1 to 0.4, or 0.1 to 0.3, or 0.1 to 0.2, or 0.15 to 0.45, or 0.15 to 0.4, or 0.15 to 0.3, or 0.15 to 0.25, or 0.15 to 0.2, or 0.2 to 0.8, or 0.2 to 0.6, or 0.2 to 0.4, or 0.2 to 0.3, or 0.25 to 0.5, or 0.25 to 0.4, or 0.25 to 0.35, or 0.25 to 0.3, or 0.3 to 0.6, or 0.3 to 0.5, or 0.3 to 0.45, or 0.3 to 0.4, or 0.3 to 0.35, or 0.5 to 0.8, or 0.5 to 0.75, or 0.5 to 0.7 dL/g;
3.39. Any of Compositions 3.30-3.38, wherein said copolymer has an average molecular weight of 5,000 to 150,000 Daltons, e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
3.40. Any of Compositions 3.30-3.39, wherein said copolymer has a molecular weight range of 5,000 to 150,000 Daltons (e.g., at least 90% of polymer molecules have a molecular weight within the range), e.g., 5,000 to 100,000 Da, or 5,000 to 75,000 Da, or 5,000 to 50,000 Da, or 5,000 to 25,000 Da, or 5,000 to 20,000 Da, or 7,000 to 20,000 Da, or 10,000 to 40,000 Da, or 10,000 to 35,000 Da, or 10,000 to 30,000 Da, or 10,000 to 25,000 Da, or 15,000 to 30,000 Da, or 15,000 to 25,000 Da, or 20,000 to 50,000 Da, or 20,000 to 45,000 Da, or 20,000 to 40,000 Da, or 25,000 to 75,000 Da, or 25,000 to 60,000 Da, or 25,000 to 50,000 Da, or 25,000 to 45,000 Da;
3.41. Any of Compositions 3.30-3.40, wherein the composition comprises said copolymer in a net amount of 10 to 80% by weight of the composition, e.g., 10% to 75%, or 10% to 70%, or 10% to 65%, or 10% to 60%, or 10% to 55%, 10 to 50%, or 10 to 45%, or 10 to 40%, 15% to 75%, or 15% to 70%, or 15% to 65%, or 15% to 60%, or 15% to 55%, or 15 to 50%, or 15 to 45%, or 15 to 40%, or 15 to 35%, 20% to 75%, or 20% to 70%, or 20% to 65%, or 20% to 60%, or 20% to 55%, or 20 to 50%, or 20 to 45%, or 20 to 40%, or 20 to 35%, 25% to 75%, or 25% to 70%, or 25% to 65%, or 25% to 60%, or 25% to 55%, or 25 to 50%, or 25 to 45%, or 25 to 40%, or 25 to 35%, or 30% to 75%, or 30% to 70%, or 30% to 65%, or 30% to 60%, or 30% to 55%, or 30 to 50%, or 30 to 45%, or 30 to 40%, or 30 to 35%, or 35 to 50%, or 35 to 45%, or 35 to 40%, or about 30%, or about 35%, or about 45%, by weight of the composition;
3.42. Any of Compositions 3.30-3.41, wherein the composition comprises both a diblock polyester-poly(ethylene glycol) copolymer and a triblock polyester-poly(ethylene glycol) copolymer, each independently as described above, e.g., in a molar ratio from 6:1 to 1:6, e.g., 5:1 to 1:5, or 4:1 to 1:4, or 3:1 to 1:3, or 2:1 to 1:2, or about 4:1, or about 1:4, or about 3:1, or about 1:3, or about 2:1, or about 1:2, or about 1:1, or about 3:5, or about 5:3;
3.43. Any of Compositions 3.30-3.41, wherein the composition comprises both a diblock poly(lactic acid)-PEG copolymer and a triblock poly(lactic acid)-PEG copolymer, each independently as described above;
3.44. Composition 3.43, wherein the diblock poly(lactic acid)-PEG copolymer has a methyl ether end group (e.g., a methoxy-PEG block);
3.45. Composition 3, or any of 3.1-3.44, wherein the composition further comprises an anti-oxidant, e.g., selected from one or more of tocopherol, butylated hydroxytoluene (BHT), propyl gallate (PG, e.g., n-propyl gallate), ascorbic acid, butylated hydroxyanisole (BHA), tert-butylhydroquinone (TBHQ), carotenoids, glutathione, sodium metabisulfite, sodium ethylenediaminetetraacetate, cysteine, methionine, sesamol, citrate (e.g., sodium citrate), and citric acid, optionally in an amount of 0.1 to 1% by weight of the composition (e.g., about 0.25%, or about 0.5%, or about 0.75%);
3.46. Composition 3, or any of 3.1-3.44, wherein the composition further comprises an anti-oxidant selected from alpha-tocopherol, butylated hydroxytoluene (BHT), propyl gallate (PG, e.g., n-propyl gallate), and butylated hydroxyanisole (BHA), in an amount of 0.1 to 1% by weight of the composition (e.g., about 0.25%, or about 0.5%, or about 0.75%);
3.47. Composition 3, or any of 3.1-3.46, wherein the composition further comprises toluenesulfonic acid, e.g., in a molar ratio of about 1:1 to 1:2 with respect to the tetradeuterolumateperone mono-tosylate, e.g., 1:1 to 1:1.5 molar ratio, or 1:1 to 1:2 molar ratio, or about a 1:1 molar ratio;
3.48. Composition 3, or any of 3.1-3.47, wherein the composition further comprises one or more additional polymers selected from polysaccharides (e.g., starches, dextrans, pectins, alginates, carrageenans, cellulose), cellulose derivatives (e.g., carboxymethyl cellulose, methylcellulose, hydroxyalkyl celluloses (e.g., hydroxyethyl cellulose, hydroxypropyl cellulose, hydroxypropyl methyl cellulose), synthetic polymers (e.g., polyvinylpyrrolidones, polyethylene oxide and/or polypropylene oxide polymers and copolymers (e.g., poloxamers, such as poloxamer 188), polyacrylate polymers (e.g., carbopols), and polyamide polymers;
3.49. Composition 3.48, wherein the composition comprises one or more of said additional polymers each in an amount of 1 to 25% by weight of the composition, e.g., from 1 to 20%, or 1 to 15%, or 1 to 10%, or 1 to 5%, or 5 to 20%, or 5 to 15%, or 5 to 10%, or 10 to 20%, or 10 to 15%, or 15 to 20%, by weight of the composition;
3.50. Composition 3, or any of 3.1-3.49, wherein the composition further comprises one or more other excipients selected from inorganic or organic acids (e.g., citric acid, lactic acid, malic acid, gluconic acid, benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid) and their salts (e.g., sodium, potassium, calcium, magnesium, lithium, ammonium salts of aforementioned acids), inorganic or organic bases (e.g., alkali metal or alkaline earth metal carbonates, bicarbonates, hydroxide, oxides), anionic surfactants (e.g., sodium lauryl sulfate, sodium laureth sulfate, sodium dodecylbenzenesulfonate, sodium lauroyl sarcosinate, sodium stearate), cationic surfactants (e.g., benzalkonium halides, cetylpyridinium halides, cetrimonium halides, benzethonium halides), zwitterionic surfactants (e.g., cocamidoalkyl betaines, such as cocamidopropyl betaine), nonionic surfactants (e.g., fatty alcohol ethoxylates (e.g., polyethylene glycol polydodecyl ethers)), sorbitan esters (e.g., sorbitan monolaurate, sorbitan monooleate, sorbitan monopalmitate, sorbitan monostearate, sorbitan tristearate), and polyethoxylated sorbitan esters (e.g., polysorbate 20, polysorbate 40, polysorbate 60, polysorbate 80);
3.51. Composition 3.50, wherein the composition comprises one or more of said additional excipients each in an amount of 0.1 to 10% by weight of the composition, e.g., from 0.1 to 8%, or 0.1 to 6%, or 0.1 to 5%, or 0.1 to 3%, or 0.1 to 2.5%, or 0.1 to 2%, or 0.1 to 1.5%, or 0.1 to 1%, or 0.1 to 0.5%, by weight of the composition;
3.52. Composition 3, or any of 3.1-3.51, wherein the polymeric vehicle consists of the one or more of a poly(lactic acid) polymer, a poly(glycolic acid) polymer, and a poly(lactic-co-glycolic acid) copolymer and the solvent (e.g., N-methyl-2-pyrrolidone or DMSO);
3.53. Composition 3, or any of 3.1-3.52, wherein the composition consists essentially of tetradeuterolumateperone (e.g., tetradeuterolumateperone free base), the one or more of a poly(lactic acid) polymer, a poly(glycolic acid) polymer, and a poly(lactic-co-glycolic acid) copolymer and the solvent (e.g., N-methyl-2-pyrrolidone or DMSO), and optionally an anti-oxidant (e.g., n-propyl gallate);
3.54. Composition 3, or any of 3.1-3.52, wherein the composition consists essentially of tetradeuterolumateperone (e.g., tetradeuterolumateperone free base), poly(lactic acid) polymer and/or poly(lactic-co-glycolic acid) copolymer, and the solvent (e.g., N-methyl-2-pyrrolidone or DMSO), and optionally an anti-oxidant (e.g., n-propyl gallate);
3.55. Composition 3, or any of 3.1-3.52, wherein the composition consists essentially of tetradeuterolumateperone (e.g., tetradeuterolumateperone free base), poly(lactic acid) polymer, and the solvent (e.g., N-methyl-2-pyrrolidone or DMSO), and optionally an anti-oxidant (e.g., n-propyl gallate);
3.56. Composition 3, or any of 3.1-3.52, wherein the composition consists essentially of tetradeuterolumateperone (e.g., tetradeuterolumateperone free base), poly(lactic-co-glycolic acid) copolymer, and the solvent (e.g., N-methyl-2-pyrrolidone or DMSO), and optionally an anti-oxidant (e.g., n-propyl gallate);
3.57. Any one of Compositions 3.52-3.56, wherein the amount of tetradeuterolumateperone (e.g., tetradeuterolumateperone free base) is 10-40% by weight of the composition, e.g., 15%, 20%, 25%, 30%, or 35%;
3.58. Composition 3, or any of 3.1-3.57, wherein the composition consists essentially of 10-40% tetradeuterolumateperone (e.g., tetradeuterolumateperone free base), 15-50% poly(lactic acid) polymer and/or poly(lactic-co-glycolic acid) copolymer (e.g., PLGA 65:35 or 80:20), and balance solvent (e.g., N-methyl-2-pyrrolidone or DMSO), and optionally an anti-oxidant (e.g., n-propyl gallate), each measured by weight of the composition;
3.59. Composition 3, or any of 3.1-3.57, wherein the composition consists essentially of 20-40% tetradeuterolumateperone (e.g., tetradeuterolumateperone free base), 20-30% poly(lactic acid) polymer and/or poly(lactic-co-glycolic acid) copolymer, and balance solvent (e.g., N-methyl-2-pyrrolidone or DMSO), and optionally an anti-oxidant (e.g., n-propyl gallate), each measured by weight of the composition;
3.60. Composition 3, or any of 3.1-3.57, wherein the composition consists essentially of 20-30% tetradeuterolumateperone (e.g., tetradeuterolumateperone free base), 25-35% poly(lactic acid) polymer and/or poly(lactic-co-glycolic acid) copolymer, and balance solvent (e.g., N-methyl-2-pyrrolidone or DMSO), and optionally an anti-oxidant (e.g., n-propyl gallate), each measured by weight of the composition;
3.61. Composition 3.60, wherein the composition comprises about 20% tetradeuterolumateperone (e.g., tetradeuterolumateperone free base) and 28-32% polymer (e.g., about 30% polymer), or wherein the composition comprises about 25% tetradeuterolumateperone (e.g., tetradeuterolumateperone free base) and 30-35% polymer (e.g., about 33% polymer), or wherein the composition comprises about 30% tetradeuterolumateperone (e.g., tetradeuterolumateperone free base) and 25-30% polymer (e.g., about 27% polymer);
3.62. Any of Compositions 3.52-3.61, wherein the polymer has an inherent viscosity of 0.1 to 0.5 dl/g, e.g., 0.1 to 0.4 or 0.1 to 0.3, or 0.2 to 0.5, or 0.2 to 0.4;
3.63. Composition 3, or any of 3.1-3.62, wherein the composition has no added water and comprises not more than a trace amount of water (e.g., less than 1% by weight, or less than 0.5% by weight, or less than 0.1% by weight, or less than 0.05% by weight);
3.64. Composition 3, or any of 3.1-3.63, wherein the tetradeuterolumateperone (e.g., the tetradeuterolumateperone free base or tetradeuterolumateperone tosylate) is suspended in the nonaqueous liquid polymeric vehicle (e.g., to form an emulsion);
3.65. Composition 3, or any of 3.1-3.63, wherein the tetradeuterolumateperone (e.g., the tetradeuterolumateperone free base or tetradeuterolumateperone tosylate) is dissolved in the nonaqueous liquid polymeric vehicle (e.g., to form a solution, such as a homogenous solution);
3.66. Composition 3, or any of 3.1-3.65, wherein the composition is a suspension or emulsion; 3.67. Composition 3, or any of 3.1-3.65, wherein the composition is a solution, e.g., a homogenous solution;
3.68. Composition 3, or any of 3.1-3.67, wherein the composition has a viscosity of 1 to 100 centiPoise (cP), e.g., 1 to 75 cP, or 1 to 70 cP, or 1 to 65 cP, or 1 to 60 cP, or 1 to 50 cP, or 1 to 40 cP, or 1 to 30 cP, or 1 to 20 cP, or 1 to 15 cP, or 1 to 10 cP, or 5 to 60 cP, or 5 to 50 cP, or 5 to 40 cP, or 5 to 30 cP, or 5 to 20 cP, or 5 to 10 cP, or 10 to 60 cP, or 10 to 50 cP, or 10 to 40 cP, or 10 to 30 cP, or 10 to 20 cP, or 20 to 60 cP, or 20 to 50 cP, or 20 to 40 cP, or 20 to 30 cP, or 30 to 60 cP, or 30 to 50 cP, or 30 to 40 cP, or 30 to 60 cP, or 40 to 50 cP, or 50 to 60 cP;
3.69. Composition 3, or any of 3.1-3.68, wherein the composition has a viscosity of 50 to 2000 centiPoise (cP), e.g., 100 to 1100 cP, or 100 to 1000 cP, or 100 to 900 cP, or 100 to 800 cP, or 100 to 700 cP, or 100 to 600 cP, or 100 to 500 cP, or 100 to 400 cP, or 1 to 300 cP, or 1 to 200 cP, or 200 to 1000 cP, or 200 to 900 cP, or 200 to 800 cP, or 200 to 700 cP, or 200 to 600 cP, or 200 to 500 cP, or 200 to 400 cP, or 200 to 300 cP, or 300 to 1000 cP, or 300 to 900 cP, or 300 to 800 cP, or 300 to 700 cP, or 300 to 600 cP, or 300 to 500 cP, or 300 to 400 cP, or 400 to 1000 cP, or 400 to 900 cP, or 400 to 800 cP, or 400 to 700 cP, or 400 to 600 cP, or 400 to 500 cP, or 500 to 1000 cP, or 500 to 900 cP, or 500 to 800 cP, or 500 to 700 cP, or 500 to 600 cP, or 600 to 1000 cP, or 600 to 900 cP, or 600 to 800 cP, or 600 to 700 cP, or 700 to 1000 cP, or 700 to 900 cP, or 700 to 800 cP, or 800 to 1000 cP, or 800 to 900 cP, or 900 to 1000 cP, or about 300 cP, or about 400 cP, or about 500 cP, or about 600 cP, or about 700 cP, or about 800 cP;
3.70. Composition 3, or any of 3.1-3.69, wherein the composition has a density of 0.5 to 2.0 g/mL, e.g., about 0.6 to 1.6 g/mL, or 0.8 to 1.4 g/mL, or 0.9 to 1.3 g/mL, or 1.0 to 1.2 g/mL, or 1.05-1.15 g/mL, or about 1.1 g/mL;
3.71. Composition 3, or any of 3.1-3.70, wherein the composition is formulated for intramuscular injection;
3.72. Composition 3, or any of 3.1-3.70, wherein the composition is formulated for subcutaneous injection;
3.73. Composition 3, or any of 3.1-3.72, wherein after injection of the composition into the body (e.g., body tissue, such as muscle tissue), the composition solidifies to form a gel;
3.74. Composition 3, or any of 3.1-3.72, wherein after injection of the composition into the body (e.g., body tissue, such as muscle tissue), the solvent of the composition (e.g., N-methyl-2-pyrrolidone) is rapidly (e.g., less than 3 days) dispersed into the body tissues leaving a polymeric depot which release the tetradeuterolumateperone;
3.75. Composition 3.74, wherein the polymeric depot comprises from 15% to 50% by weight of tetradeuterolumateperone (measured as the free base equivalent) upon the completion of solvent dispersion, e.g., 20 to 50%, or 25 to 50%, or 25 to 45%, or 30 to 50%, or 30 to 45%, or about 20%, or about 25%, or about 30%, or about 35%;
3.76. Composition 3, or any of 3.1-3.75, wherein the tetradeuterolumateperone is in the form of a salt having low aqueous solubility, e.g., as described in any embodiment disclosed in WO 2024/030835, the contents of which are hereby incorporated by reference in its entirety;
3.77. Composition 3.76, wherein the tetradeuterolumateperone is in the form of a salt, e.g., a solid, crystalline salt, having an aqueous solubility of less than 2 mg/mL at pH 7, or less than 1 mg/mL at pH 7.4;
3.78. Composition 3.76 or 3.77, wherein the tetradeuterolumateperone is in the form of a salt, e.g., a solid, crystalline salt, selected from a 4-octylbenzenesulfonic acid addition salt, a 4-tert-butylbenzenesulfonic acid addition salt, a 4-propylbenzenesulfonic acid addition salt, a 4-ethylbenzenesulfonic acid addition salt, a 2-naphthalenesulfonic acid addition salt wherein the salt is a solvate, a solid crystalline benzenesulfonic acid addition salt characterized by a DSC thermogram lacking an endothermic event at 172-176° C., and an alkylsulfonic acid addition salt (e.g., a pentane-1-sulfonate, or a heptane-1-sulfonate);
3.79. Composition 3.76, wherein the tetradeuterolumateperone is in the form of a salt, e.g., a solid, crystalline salt, which is an acid addition salt with a benzenesulfonic acid substituted by one, two, or three groups R, wherein each R is independently a C1-12alkyl group, provided that the acid is not p-toluenesulfonic acid, 4-ethylbenzenesulfonic acid, 4-propylbenzenesulfonic acid, 4-t-butylbenzenesulfonic acid, or 4-octylbenzenesulfonic acid;
3.80. Composition 3.78, wherein the tetradeuterolumateperone is in the form of a salt, e.g., a solid, crystalline salt, which is a 4-octylbenzenesulfonic acid addition salt (e.g., 1:1 molar ratio of tetradeuterolumateperone to 4-octylbenzenesulfonic acid);
3.81. Composition 3, or any of 3.1-3.80, wherein the composition comprises the tetradeuterolumateperone, in a net concentration of 50 to 1000 mg/mL, measured as the amount of tetradeuterolumateperone free base, e.g., 50 to 500 mg/mL, 50 to 400 mg/mL, 50 to 350 mg/mL, 50 to 300 mg/mL, 50 to 250 mg/mL, 50 to 200 mg/mL, 50 to 150 mg/mL, 50 to 100 mg/mL, 50 to 75 mg/mL, 75 to 100 mg/mL, 100 to 125 mg/mL, 125 to 150 mg/mL, 100 to 400 mg/mL, 100 to 350 mg/mL, 100 to 300 mg/mL, 100 to 250 mg/mL, 100 to 200 mg/mL, 100 to 150 mg/mL, 150 to 500 mg/mL, 150 to 400 mg/mL, 150 to 350 mg/mL, 150 to 300 mg/mL, 150 to 250 mg/mL, 150 to 200 mg/mL, 200 to 500 mg/mL, 200 to 400 mg/mL, 200 to 350 mg/mL, 200 to 300 mg/mL, 200 to 250 mg/mL, 250 to 500 mg/mL, 250 to 400 mg/mL, 250 to 350 mg/mL, 250 to 300 mg/mL, 300 to 500 mg/mL, 300 to 400 mg/mL, 300 to 350 mg/mL, or 400 to 500 mg/mL;
3.82. Composition 3, or any of 3.1-3.81, wherein the composition is intended to be administered once per week, once every 2 weeks, once every 3 weeks, once every 4 weeks, once every 6 weeks, once every 2 months, once every 3 months, once every 4 months, once every 5 months, or once every 6 months, e.g., once per month;
3.83. Composition 3, or any of 3.1-3.82, wherein the composition, after intramuscular or subcutaneous injection, releases the tetradeuterolumateperone over a period of at least 1 week, 2 weeks, 3 weeks, or 4 weeks, maintaining a plasma concentration of tetradeuterolumateperone of at least 5 ng/ml, e.g., at least 10 ng/ml or at least 20 ng/ml, or 5-30 ng/ml, or 10-30 ng/ml, or 5-20 ng/mL, or 10-20 ng/ml;
3.84. Composition 3, or any of 3.1-3.83, wherein the composition, after intramuscular or subcutaneous injection, releases the tetradeuterolumateperone over a period of at least 6 weeks maintaining a plasma concentration of tetradeuterolumateperone of at least 5 ng/mL, e.g., at least 10 ng/ml or at least 20 ng/ml, or 5-30 ng/ml, or 10-30 ng/ml, or 5-20 ng/ml, or 10-20 ng/ml;
3.85. Composition 3, or any of 3.1-3.84, wherein the composition, after intramuscular or subcutaneous injection, releases the tetradeuterolumateperone over a period of at least 8 weeks maintaining a plasma concentration of tetradeuterolumateperone of at least 5 ng/mL, e.g., at least 10 ng/mL or at least 20 ng/mL, or 5-30 ng/ml, or 10-30 ng/ml, or 5-20 ng/mL, or 10-20 ng/ml;
3.86. Composition 3, or any of 3.1-3.85, wherein the composition, after intramuscular or subcutaneous injection, releases the tetradeuterolumateperone over a period of at least 10 weeks maintaining a plasma concentration of tetradeuterolumateperone of at least 5 ng/ml, e.g., at least 10 ng/mL or at least 20 ng/mL, or 5-30 ng/ml, or 10-30 ng/ml, or 5-20 ng/mL, or 10-20 ng/ml;
3.87. Composition 3, or any of 3.1-3.86, wherein the composition, after intramuscular or subcutaneous injection, releases the tetradeuterolumateperone over a period of at least 12 weeks maintaining a plasma concentration of tetradeuterolumateperone of at least 5 ng/mL, e.g., at least 10 ng/mL or at least 20 ng/mL, or 5-30 ng/ml, or 10-30 ng/mL, or 5-20 ng/mL, or 10-20 ng/ml;
3.88. Composition 3, or any of 3.1-3.87, wherein the composition, after intramuscular or subcutaneous injection, releases the tetradeuterolumateperone at a rate to provide not more than 100 ng/mL plasma concentration of tetradeuterolumateperone at any time after 9 days after injection, e.g., not more than 85 ng/mL, or not more than 75 ng/ml, or not more than 70 ng/mL, or not more than 65 ng/mL, or not more than 60 ng/mL, or not more than 50 ng/mL, or not more than 40 ng/mL, or not more than 30 ng/ml, or not more than 20 ng/ml;
3.89. Composition 3, or any of 3.1-3.88, wherein the composition, after intramuscular or subcutaneous injection, releases the tetradeuterolumateperone at a rate to provide not more than 300 ng/mL plasma concentration of tetradeuterolumateperone at any time within 72 hours after injection, e.g., not more than 250 ng/ml, or not more than 200 ng/mL, or not more than 150 ng/mL, or not more than 125 ng/ml, or not more than 100 ng/mL, or not more than 75 ng/mL, or not more than 50 ng/mL, or not more than 25 ng/ml;
3.90. Composition 3, or any of 3.1-3.89, wherein the composition, after intramuscular or subcutaneous injection (e.g., of a 100 mg dose), provides a steady-state plasma concentration of tetradeuterolumateperone of not more than 60 ng/ml, e.g., not more than 55 ng/mL, or not more than 50 ng/ml, or not more than 45 ng/ml, or not more than 40 ng/ml;
3.91. Composition 3, or any of 3.1-3.89, wherein the composition, after intramuscular or subcutaneous injection (e.g., of a 100 mg dose), provides a steady-state plasma concentration of tetradeuterolumateperone of 2.5-30 ng/ml, e.g., 2.5 to 25 ng/mL, or 2.5 to 20 ng/ml, or 2.5 to 15 ng/ml or 2.5 to 10 ng/ml, or 2.5 to 5 ng/ml;
3.92. Composition 3, or any of 3.1-3.89, wherein the composition, after intramuscular or subcutaneous injection (e.g., of a 100 mg dose), provides a maximal plasma concentration (Cmax) of tetradeuterolumateperone of 10 to 25 ng/ml, e.g., 10-15 ng/mL, or 15 to 20 ng/mL, or 20 to 25 ng/mL, or 15 to 25 ng/ml or 10 to 20 ng/ml;
3.93. Composition 3, or any of 3.1-3.92, wherein the composition, after intramuscular or subcutaneous injection, releases not more than 10% of the total amount of tetradeuterolumateperone within the first 8 hours after injection, e.g., not more than 8%, or not more than 6% or not more than 5%;
3.94. Composition 3, or any of 3.1-3.93, wherein the composition comprises, or consists of, tetradeuterolumateperone free base or tetradeuterolumateperone monotosylate in an amount of 10-15% (e.g., about 12%), PLA or PLGA copolymer (e.g., PLGA 50:50, PLGA 65:35, PLGA 75:25, or PLGA 80:20) in an amount of 25-40% (e.g., about 33%), and N-methyl pyrrolidine (NMP) as the balance (e.g., 50-70%, or about 55%), optionally wherein the three components have a weight ratio of about 1:2.6:4.4;
3.95. Composition 3, or any of 3.1-3.93, wherein the composition comprises, or consists of, tetradeuterolumateperone free base or tetradeuterolumateperone monotosylate in an amount of 20-40% (e.g., about 30%), PLA or PLGA copolymer (e.g., PLGA 50:50, PLGA 65:35, PLGA 75:25, or PLGA 80:20) in an amount of 20-40% (e.g., about 30%), and N-methyl pyrrolidine (NMP) as the balance (e.g., 30-60%, or about 40%), optionally wherein the three components have a weight ratio of about 1:1:1.3;
3.96. Composition 3, or any of 3.1-3.93, wherein the composition comprises, or consists of, tetradeuterolumateperone free base or tetradeuterolumateperone monotosylate in an amount of 10-30% (e.g., about 20%), PLA or PLGA copolymer (e.g., PLGA 50:50, PLGA 65:35, PLGA 75:25, or PLGA 80:20) in an amount of 20-40% (e.g., about 30%), and N-methyl pyrrolidine (NMP) as the balance (e.g., 30-70%, or about 50%), optionally wherein the three components have a weight ratio of about 1:1.5:2.5;
3.97. Composition 3, or any of 3.1-3.93, wherein the composition comprises, or consists of, tetradeuterolumateperone free base or tetradeuterolumateperone monotosylate in an amount of 15-35% (e.g., about 25%), PLA or PLGA copolymer (e.g., PLGA 50:50, PLGA 65:35, PLGA 75:25, or PLGA 80:20) in an amount of 15-35% (e.g., about 25%), and N-methyl pyrrolidine (NMP) as the balance (e.g., 30-70%, or about 50%), optionally wherein the three components have a weight ratio of about 1:1:2;
3.98. Composition 3, or any of 3.1-3.93, wherein the composition comprises, or consists of, tetradeuterolumateperone free base or tetradeuterolumateperone monotosylate in an amount of 15-25% (e.g., about 25%), PLA or PLGA copolymer (e.g., PLGA 50:50, PLGA 65:35, PLGA 75:25, or PLGA 80:20) in an amount of 10-30% (e.g., about 17%), and N-methyl pyrrolidine (NMP) as the balance (e.g., 40-80%, or about 58%), optionally wherein the three components have a weight ratio of about 1:0.67:2.3;
3.99. Composition 3, or any of 3.1-3.93, wherein the composition comprises, or consists of, tetradeuterolumateperone free base or tetradeuterolumateperone monotosylate in an amount of 20-40% (e.g., about 30%), PLA or PLGA copolymer (e.g., PLGA 50:50, PLGA 65:35, PLGA 75:25, or PLGA 80:20) in an amount of 10-30% (e.g., about 20%), and N-methyl pyrrolidine (NMP) as the balance (e.g., 30-70%, or about 50%), optionally wherein the three components have a weight ratio of about 1:0.67:1.7;
3.100. Composition 3, or any of 3.1-3.93, wherein the composition comprises, or consists of, tetradeuterolumateperone free base or tetradeuterolumateperone monotosylate in an amount of 20-40% (e.g., about 30%), PLA or PLGA copolymer (e.g., PLGA 50:50, PLGA 65:35, PLGA 75:25, or PLGA 80:20) in an amount of 15-35% (e.g., about 27%), and N-methyl pyrrolidine (NMP) as the balance (e.g., 20-60%, or about 43%), optionally wherein the three components have a weight ratio of about 1:0.9:1.4;
3.101. Composition 3, or any of 3.1-3.93, wherein the composition comprises, or consists of, tetradeuterolumateperone free base or tetradeuterolumateperone monotosylate in an amount of 15-35% (e.g., about 25%), PLA or PLGA copolymer (e.g., PLGA 50:50, PLGA 65:35, PLGA 75:25, or PLGA 80:20) in an amount of 25-45% (e.g., about 33%), and N-methyl pyrrolidine (NMP) as the balance (e.g., 20-60%, or about 40%), optionally wherein the three components have a weight ratio of about 1:1.3:1.7;
3.102. Any of Compositions 3.94-3.101, wherein the tetradeuterolumateperone is tetradeuterolumateperone free base;
3.103. Any of Compositions 3.94-3.102, wherein the polymer is PLA (e.g., having an inherent viscosity of 0.1-0.3 dL/g, and optionally with acid end groups);
3.104. Any of Compositions 3.94-3.102, wherein the polymer is PLA (e.g., having an inherent viscosity of 0.1-0.3 dL/g, and optionally with ester end groups);
3.105. Any of Compositions 3.94-3.102, wherein the polymer is PLA (e.g., having an inherent viscosity of 0.15-0.19 dL/g, and optionally with acid end groups);
3.106. Any of Compositions 3.94-3.102, wherein the polymer is PLA (e.g., having an inherent viscosity of 0.30-0.35 dL/g, and optionally with acid end groups);
3.107. Any of Compositions 3.94-3.102, wherein the polymer is PLA (e.g., having an inherent viscosity of 0.32-0.38 dL/g, and optionally with ester end groups);
3.108. Any of Compositions 3.94-3.102, wherein the polymer is PLA (e.g., having an inherent viscosity of 0.18-0.24 dL/g, and optionally with ester end groups);
3.109. Any of Compositions 3.94-3.102, wherein the polymer is PLGA 80:20 (e.g., having an inherent viscosity of 0.2-0.4 dL/g, and optionally with acid end groups);
3.110. Any of Compositions 3.94-3.102, wherein the polymer is PLGA 80:20 (e.g., having an inherent viscosity of 0.2-0.4 dL/g, and optionally with ester end groups);
3.111. Any of Compositions 3.94-3.102, wherein the polymer is PLGA 80:20 (e.g., having an inherent viscosity of 0.25-0.29 dL/g, and optionally with acid end groups);
3.112. Any of Compositions 3.94-3.102, wherein the polymer is PLGA 80:20 (e.g., having an inherent viscosity of 0.20-0.30 dL/g, and optionally with acid end groups);
3.113. Any of Compositions 3.94-3.102, wherein the polymer is PLGA 75:25 (e.g., having an inherent viscosity of 0.15-0.25 dL/g, and optionally with acid end groups);
3.114. Any of Compositions 3.94-3.102, wherein the polymer is PLGA 75:25 (e.g., having an inherent viscosity of 0.30-0.40 dL/g, and optionally with acid end groups);
3.115. Any of Compositions 3.94-3.102, wherein the polymer is PLGA 75:25 (e.g., having an inherent viscosity of 0.35-0.45 dL/g, and optionally with acid end groups);
3.116. A single-use kit comprising a vial containing the Composition 3, or any of 3.1-3.115, and a syringe with a needle (e.g., permanently attached or detachable), with instructions for use;
3.117. A multiple-use kit comprising one or more vials containing the Composition 3, or any of 3.1-3.115, and one or more syringes with needles (e.g., permanently attached or detachable), with instructions for use;
3.118. Kit 3.116 or 3.117, wherein the needle is an 18 to 25 Gauge needle, e.g., 19 to 25 Gauge, or 20 to 25 Gauge, or 21 to 25 Gauge, or 23 to 25 Gauge;
3.119. Any one of kits 3.116-3.118, wherein the needle is a 1-1.5-inch length needle, e.g., 1.0 inch or 1.5 inch, optionally wherein the kit comprises a needle spacer between the needle and the syringe;
3.120. A pre-filled syringe (e.g., an automatic syringe) comprising Composition 3, or any of 3.1-3.115, optionally wherein the syringe needle is an 18 to 25 Gauge needle, e.g., 19 to 25 Gauge, or 20 to 25 Gauge, or 21 to 25 Gauge, or 23 to 25 Gauge;
3.121. Syringe 3.120, wherein the needle is a 1-1.5-inch length needle, e.g., 1.0 inch or 1.5 inch, optionally wherein a needle spacer is comprised between the needle and the syringe;
3.122. A kit comprising a pre-filled syringe (e.g., an automatic syringe) comprising Composition 3, or any of 3.1-3.115;
3.123. Kit 3.122, wherein the syringe needle is an 18 to 25 Gauge needle, e.g., 19 to 25 Gauge, or 20 to 25 Gauge, or 21 to 25 Gauge, or 23 to 25 Gauge;
3.124. Kit 3.122 or 3.123, wherein the needle is a 1-1.5-inch length needle, e.g., 1.0 inch or 1.5 inch, optionally wherein the kit comprises a needle spacer between the needle and the syringe;
3.125. Any of kits 3.116-3.124, wherein the syringe has a total volume of 0.3 to 2.0 mL, e.g., 0.3 to 1.5 mL, or 0.3 to 1.2 mL, or 0.3 to 1.0 mL;
3.126. A kit (e.g., single-use or multiple-use) comprising, in separate compartments, two compositions:
   (a) a first composition comprising tetradeuterolumateperone in free base or pharmaceutically acceptable salt form (e.g., in tosylate salt form), and, at least one of a poly(lactic acid) polymer, a poly(glycolic acid) polymer, a poly(lactic-co-glycolic acid) copolymer, and a polyester-poly(ethylene glycol) copolymer, wherein the composition does not comprise polymeric microspheres, and a second composition comprising at least one nonaqueous (e.g., organic) solvent (e.g., NMP or DMSO), optionally wherein the first composition is a dry solid, e.g., powder or granules, or an oil; or
   (b) a first composition comprising tetradeuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, optionally wherein the second composition is a dry solid, e.g., powder or granules; or
   (c) a first composition comprising tetradeuterolumateperone in free base or pharmaceutically acceptable salt form (e.g., in tosylate salt form) wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), optionally wherein the first composition is a dry solid, e.g., powder or granules, or an oil; or
   (d) a first composition comprising tetradeuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), preferably wherein the solvent of the first composition is the same as the solvent of the second composition, and optionally wherein both the first composition and the second composition are homogenous solutions;
      wherein upon combination of the first composition and the second composition, an injectable composition according to Composition 3 or any of 3.1-3.115 is formed (e.g., the tetradeuterolumateperone becomes dissolved or suspended in a nonaqueous liquid polymeric vehicle comprising the at least one nonaqueous (e.g., organic) solvent and the at least one of a poly(lactic acid) polymer, a poly(glycolic acid) polymer, a poly(lactic-co-glycolic acid) copolymer, and a polyester-poly(ethylene glycol) copolymer, and wherein the combination does not result in polymeric microspheres), optionally, wherein the injectable composition formed after combination of the first composition and the second composition is a homogenous solution;
3.127. Kit 3.126, wherein the first composition and the second composition are combined to provide an amount of tetradeuterolumateperone and polymer of 1000-2000 mg of the first composition (e.g., 1100-1800 mg, or 1200 to 1700 mg, or 1300-1400 mg, or 1100-1300 mg, or 1500-1700 mg, or about 1240 mg, or about 1350 mg, or about 1620 mg) and/or a volume of solvent of 1-3 mL (e.g., 1-2 mL, or 1-1.5 mL, or 1.5-2 mL, or 1-1.1 mL, or 1.1-1.5 mL, or 1.1-1.4 mL, or 1.2-1.4 mL, or about 1 mL, or about 1.3 mL, or about 1.45 mL), to form said injectable composition;
3.128. Kit 3.126 or 3.127, wherein the first composition comprises 200 to 1000 mg of tetradeuterolumateperone, measured as the as the amount of free base, e.g., 300 to 900 mg, or 300 to 800 mg, or 300 to 700 mg, or 300 to 600 mg, or 400 to 600 mg; and optionally wherein the second composition comprises 200 to 1000 mg of polymer or polymers, e.g., 300 to 900 mg, or 300 to 800 mg, or 300 to 700 mg, or 300 to 600 mg, or 400 to 600 mg;
3.129. Any of Kits 3.126-3.128, wherein the first composition is an oil and the second composition is a homogenous liquid (e.g., comprising or consisting of polymer and solvent, e.g., N-methyl pyrrolidone or DMSO);
3.130. Any of Kits 3.126-3.128, wherein the first composition is a dry solid, e.g. powder or granules, and the second composition is a homogenous liquid (e.g., comprising or consisting of polymer and solvent, e.g., N-methyl-2-pyrrolidone or DMSO);
3.131. Any of Kits 3.126-3.131, wherein the first composition is a homogenous liquid (e.g., comprising or consisting of tetradeuterolumateperone and solvent, e.g., N-methyl-2-pyrrolidone); and the second composition is a homogenous liquid (e.g., comprising or consisting of polymer and solvent, e.g., N-methyl-2-pyrrolidone or DMSO);
3.132. Any of Kits 3.126-3.131, wherein the kit comprises the first composition in a sealed vial, the second composition in a sealed vial, and the kit further comprises a syringe and a needle;
3.133. Kit 3.132, wherein the needle is an 18 to 25 Gauge needle, e.g., 19 to 25 Gauge, or 20 to 25 Gauge, or 21 to 25 Gauge, or 23 to 25 Gauge;
3.134. Any of kits 3.126-3.133, wherein the needle is a 1-1.5-inch length needle, e.g., 1.0 inch or 1.5 inch, optionally wherein the kit comprises a needle spacer between the needle and the syringe;
3.135. Any of kits 3.126-3.134, wherein the syringe has a total volume of 0.3 to 2.0 mL, e.g., 0.3 to 1.5 mL, or 0.3 to 1.2 mL, or 0.3 to 1.0 mL;
3.136. A pre-filled dual-compartment syringe (e.g., an automatic syringe) comprising, in separate compartments, two compositions:
   (a) a first composition comprising tetradeuterolumateperone in free base or pharmaceutically acceptable salt form (e.g., in tosylate salt form), and, at least one of a poly(lactic acid) polymer, a poly(glycolic acid) polymer, a poly(lactic-co-glycolic acid) copolymer, and a polyester-poly(ethylene glycol) copolymer, wherein the composition does not comprise polymeric microspheres, and a second composition comprising at least one nonaqueous (e.g., organic) solvent (e.g., NMP or DMSO), optionally wherein the first composition is a dry solid, e.g., powder or granules, or an oil; or
   (b) a first composition comprising tetradeuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, optionally wherein the second composition is a dry solid, e.g., powder or granules; or
   (c) a first composition comprising tetradeuterolumateperone in free base or pharmaceutically acceptable salt form (e.g., in tosylate salt form) wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), optionally wherein the first composition is a dry solid, e.g., powder or granules, or an oil; or
   (d) a first composition comprising tetradeuterolumateperone in free base or pharmaceutically acceptable salt form (e.g., in tosylate salt form) dissolved or suspended in at least one nonaqueous (e.g., organic) solvent (e.g., NMP or DMSO), wherein the composition does not comprise polymeric microspheres, and a second composition comprising at least one of a poly(lactic acid) polymer, a poly(glycolic acid) polymer, a poly(lactic-co-glycolic acid) copolymer, and a polyester-poly(ethylene glycol) copolymer, dissolved or suspended in at least one nonaqueous (e.g., organic) solvent (e.g., NMP or DMSO), preferably wherein the solvent of the first composition is the same as the solvent of the second composition, and optionally wherein both the first composition and the second composition are homogenous solutions;
      wherein upon actuation of the syringe the first composition and the second composition are combined to form (e.g., the tetradeuterolumateperone becomes dissolved or suspended in a nonaqueous liquid polymeric vehicle comprising the at least one nonaqueous (e.g., organic) solvent and the at least one of a poly(lactic acid) polymer, a poly(glycolic acid) polymer, a poly(lactic-co-glycolic acid) copolymer, and a polyester-poly(ethylene glycol) copolymer, and wherein the combination does not result in polymeric microspheres), optionally,
      wherein the injectable composition formed after combination of the first composition and the second composition is a homogenous solution;
3.137. Syringe 3.136, wherein the first composition and the second composition are combined to provide an amount of tetradeuterolumateperone and polymer of 1000-2000 mg of the first composition (e.g., 1100-1800 mg, or 1200 to 1700 mg, or 1300-1400 mg, or 1100-1300 mg, or 1500-1700 mg, or about 1240 mg, or about 1350 mg, or about 1620 mg) and/or a volume of solvent of 1-3 mL (e.g., 1-2 mL, or 1-1.5 mL, or 1.5-2 mL, or 1-1.1 mL, or 1.1-1.5 mL, or 1.1-1.4 mL, or 1.2-1.4 mL, or about 1 mL, or about 1.3 mL, or about 1.45 mL), to form said injectable composition;
3.138. Syringe 3.136 or 3.137, wherein the first composition comprises 200 to 1000 mg of tetradeuterolumateperone, measured as the as the amount of free base, e.g., 300 to 900 mg, or 300 to 800 mg, or 300 to 700 mg, or 300 to 600 mg, or 400 to 600 mg; and optionally wherein the second composition comprises 200 to 1000 mg of polymer or polymers, e.g., 300 to 900 mg, or 300 to 800 mg, or 300 to 700 mg, or 300 to 600 mg, or 400 to 600 mg;
3.139. Any of Syringes 3.136-3.138, wherein the first composition is an oil and the second composition is a homogenous liquid (e.g., comprising or consisting of polymer and solvent, e.g., N-methyl pyrrolidone or DMSO);
3.140. Any of Syringes 3.136-3.138, wherein the first composition is a dry solid, e.g. powder or granules, and the second composition is a homogenous liquid (e.g., comprising or consisting of polymer and solvent, e.g., N-methyl-2-pyrrolidone or DMSO);
3.141. Any of Syringes 3.136-3.138, wherein the first composition is a homogenous liquid (e.g., comprising or consisting of tetradeuterolumateperone and solvent, e.g., N-methyl-2-pyrrolidone); and the second composition is a homogenous liquid (e.g., comprising or consisting of polymer and solvent, e.g., N-methyl-2-pyrrolidone or DMSO);
3.142. Any of syringes 3.136-3.141, wherein the needle is a 1-1.5-inch length needle, e.g., 1.0 inch or 1.5 inch, optionally wherein the kit comprises a needle spacer between the needle and the syringe;
3.143. Any of syringes 3.136-3.142, wherein the syringe has a total volume of 0.3 to 2.0 mL, e.g., 0.3 to 1.5 mL, or 0.3 to 1.2 mL, or 0.3 to 1.0 mL;
3.144. Any preceding Composition, Kit or Syringe, wherein the administration dose of the composition, kit or syringe is 200 to 1000 mg of tetradeuterolumateperone, measured as the free base equivalent, delivered in a single injection, e.g., 300 to 900 mg, or 300 to 800 mg, or 300 to 700 mg, or 300 to 600 mg, or 400 to 600 mg;
3.145. Any preceding Composition, Kit or Syringe, wherein the composition is stable for at least 6 months, e.g., at least 12 months, at least 18 months, or at least 24 months, e.g., as measured by one or more of: stability of color and appearance (e.g., color and appearance are unchanged), stability of tetradeuterolumateperone assay (e.g., by HPLC), stability of particle size distribution, stability of viscosity, stability of water content, and stability of concentration of degradation products or impurities (e.g., wherein “stable” refers to a change from initial condition of not more than 5% of the property).

Preservative agents may be added to prevent the growth of microorganisms such as bacteria, yeasts and fungi in liquid formulations, which are likely to be used repeatedly. Suitable preservatives should be physicochemical stable and effective in the desired pH range. Examples of preservative agents include ethanol, methylparaben, propylparaben and benzyl alcohol.

In some embodiments, the solid dosage forms of the present disclosure include one or more anti-oxidants to guard against degradation of the active. Examples of antioxidants include propyl gallate, ascorbyl palmitate, ascorbic acid, t-butylhydroquinone (TBHQ), butylated hydroxyanisole (BHA), butylated hydroxytoluene (BHT), tocopherols, tocotrienols, sodium sulfite, sodium metabisulfite, beta-carotene, citric acid and EDTA.

Suitable forms of lumateperone or deuterolumateperone or tetradeuterolumateperone include the free base form, including amorphous solid dispersions thereof, pharmaceutically acceptable salt forms, including amorphous solid dispersions and crystal forms thereof, and pharmaceutically acceptable co-crystal forms. Amorphous solid dispersion forms of lumateperone and deuterolumateperone are disclosed in US 2019/0192511 and US 2019/0388418, the contents of each of which are hereby incorporated by reference in their entireties.

Unless otherwise indicated, the term “pharmaceutically acceptable salt” includes acid addition salts between lumateperone or deuterolumateperone or tetradeuterolumateperone and any pharmaceutically acceptable acid (e.g., Bronsted acid) in any molar ratio permitted by the structure of the acid. For example, “pharmaceutically acceptable salt form” of lumateperone or deuterolumateperone or tetradeuterolumateperone includes the mono-hydrochloride, the di-hydrochloride, the tri-hydrochloride, the mono-tosylate, the di-tosylate and the tri-tosylate, or any mixtures thereof. In some embodiments, the lumateperone or deuterolumateperone or tetradeuterolumateperone salt is a crystalline solid (e.g., a salt crystal). In some embodiments, the lumateperone or deuterolumateperone or tetradeuterolumateperone may exist as a co-crystal, i.e., lumateperone or deuterolumateperone free base co-crystallized with a second species.

Pharmaceutically acceptable salt and co-crystal forms of lumateperone or deuterolumateperone or tetradeuterolumateperone include all those forms disclosed in the following: U.S. Pat. Nos. 8,648,077, 9,199,995, 9,586,960, 10,654,854, 10,688,097, 11,014,925, 11,096,944, and RE48,825; and patent publications US 2020/247805, US 2020/0157100, US 2019/0211015 (Assia Pharm.), and WO2020/112941 (Teva Czech), and PCT/US2021/071366, the contents of each of which are hereby incorporated by reference in their entireties.

In some embodiments, the pharmaceutically acceptable salt may be a low-solubility salt, such as those described in WO2024/030835, the contents of which are hereby incorporated by reference in its entirety. Such salts are typically unsuitable for oral or intravenous administration, but may be particularly well-suited to the sustained/delayed bioabsorption required by the compositions of the present disclosure. Such salts include C2-C12alkylbenzenesulfonate salts, such as 4-octylbenzenesulfonate, 4-butylbenzenesulfonate, 4-propylbenzenesulfonate, and 4-ethylbenzenesulfonate, as well as C3-C12alkylsulfonate salts, such as pentane-1-sulfonate, hexane-1-sulfonate, and heptane-1-sulfonate, and other arylsulfonate salts, such as benzenesulfonate and 2-naphthalenesulfonate. Such salts preferably have an aqueous solubility of less than 20 mg/mL at pH 7 or pH 7.4, e.g., less than 15 mg/mL, or less than 10 mg/mL, or less than 5 mg/mL, or less than 3 mg/mL, or less than 2 mg/mL, or less than 1 mg/mL, or less than 0.5 mg/mL, or less than 0.1 mg/mL, and/or at least 0.001 mg/mL, or at least 0.01 mg/mL, or at least 0.1 mg/mL, or at least 1 mg/mL. Preferably, such salts are solid, crystalline salts. Such salts may have a molar ratio of lumateperone free base to acid counterion of about 1:1: or about 1:2. In some embodiments, the salt is a mono-4-octylbenzenesulfonate salt (e.g., in solid, crystalline form). In some embodiments, the salt is a di-4-octylbenzenesulfonate salt (e.g., in solid crystalline form).

In a fourth aspect, the present disclosure provides a process (Process 1) for the manufacture of Composition 1, or any of 1.1-1.115, wherein the process comprises the steps of:
(a) combining lumateperone, in free or pharmaceutically acceptable salt form (e.g., tosylate salt form), with at least one nonaqueous (e.g., organic) solvent (e.g., N-methyl-2-pyrrolidone or DMSO);
(b) optionally mixing, agitating, stirring, vortexing, or sonicating the resulting mixture until it is homogenous, optionally with concurrent heating;
(c) adding at least one of a poly(lactic acid) polymer, a poly(glycolic acid) polymer, and a poly(lactic-co-glycolic acid) copolymer, as described in any embodiments hereinbefore described, optionally in a nonaqueous (e.g., organic) solvent (e.g., N-methyl-2-pyrrolidone or DMSO);
(d) mixing, agitating, stirring, vortexing, or sonicating the resulting mixture until it is homogenous, optionally with concurrent heating;
(e) optionally filtering the resulting mixture, e.g., to remove undissolved matter;
(f) optionally sterilizing the resulting mixture; and
(g) optionally filling the resulting composition into vials or pre-filled syringes;
   wherein the Process does not result in the formation of polymeric microspheres.

In a particular embodiment of the fourth aspect, the present disclosure provides Process 1 wherein the steps comprise:
(a) filling lumateperone, in free or pharmaceutically acceptable salt form (e.g., tosylate salt form) into a vial or a chamber of a dual-compartment syringe, optionally with at least one nonaqueous (e.g., organic) solvent (e.g., N-methyl-2-pyrrolidone or DMSO);
(b) dissolving at least one of a poly(lactic acid) polymer, a poly(glycolic acid) polymer, a poly(lactic-co-glycolic acid) copolymer, or a polyester-poly(ethylene glycol) copolymer, as described in any embodiments hereinbefore described in least one nonaqueous (e.g., organic) solvent (e.g., N-methyl-2-pyrrolidone or DMSO), and adding the resulting solution to vials or chamber of a dual-compartment syringe;
(c) optionally sterilizing the filled vials or syringes; and
(d) packaging the vials or syringes, e.g., in a kit, for distribution to patients;
   preferably wherein mixing the contents of the vials of steps (a) and (b), or the syringe compartments of steps (a) and (b), generates a homogenous or suspension for injection.

In a fifth aspect, the present disclosure provides a process (Process 2) for the manufacture of Composition 2, or any of 2.1-2.115, wherein the process comprises the steps of:
(a) combining deuterolumateperone, in free or pharmaceutically acceptable salt form (e.g., tosylate salt form), with at least one nonaqueous (e.g., organic) solvent (e.g., N-methyl-2-pyrrolidone or DMSO);
(b) mixing, agitating, stirring, vortexing, or sonicating the resulting mixture until it is homogenous, optionally with concurrent heating;
(c) adding at least one of a poly(lactic acid) polymer, a poly(glycolic acid) polymer, and a poly(lactic-co-glycolic acid) copolymer, as described in any embodiments hereinbefore described, optionally in a nonaqueous (e.g., organic) solvent (e.g., N-methyl-2-pyrrolidone or DMSO;
(d) mixing, agitating, stirring, vortexing, or sonicating the resulting mixture until it is homogenous, optionally with concurrent heating;
(e) optionally filtering the resulting mixture, e.g., to remove undissolved matter;
(f) optionally sterilizing the resulting mixture; and
(g) optionally filling the resulting composition into vials or pre-filled syringes;
   wherein the Process does not result in the formation of polymeric microspheres.

In a particular embodiment of the fifth aspect, the present disclosure provides Process 2 wherein the steps comprise:
(a) filling deuterolumateperone, in free or pharmaceutically acceptable salt form (e.g., tosylate salt form) into a vial or a chamber of a dual-compartment syringe, optionally with at least one nonaqueous (e.g., organic) solvent (e.g., N-methyl-2-pyrrolidone or DMSO);
(b) dissolving at least one of a poly(lactic acid) polymer, a poly(glycolic acid) polymer, a poly(lactic-co-glycolic acid) copolymer, or a polyester-poly(ethylene glycol) copolymer, as described in any embodiments hereinbefore described in least one nonaqueous (e.g., organic) solvent (e.g., N-methyl-2-pyrrolidone or DMSO), and adding the resulting solution to vials or chamber of a dual-compartment syringe;
(c) sterilizing the filled vials or syringes; and
(d) packaging the vials or syringes, e.g., in a kit, for distribution to patients;
   preferably wherein mixing the contents of the vials of steps (a) and (b), or the syringe compartments of steps (a) and (b), generates a homogenous or suspension for injection.

In a sixth aspect, the present disclosure provides a process (Process 3) for the manufacture of Composition 3, or any of 3.1-3.115, wherein the process comprises the steps of:
(a) combining tetradeuterolumateperone, in free or pharmaceutically acceptable salt form (e.g., tosylate salt form), with at least one nonaqueous (e.g., organic) solvent (e.g., N-methyl-2-pyrrolidone or DMSO);
(b) optionally mixing, agitating, stirring, vortexing, or sonicating the resulting mixture until it is homogenous, optionally with concurrent heating;
(c) adding at least one of a poly(lactic acid) polymer, a poly(glycolic acid) polymer, and a poly(lactic-co-glycolic acid) copolymer, as described in any embodiments hereinbefore described, optionally in a nonaqueous (e.g., organic) solvent (e.g., N-methyl-2-pyrrolidone or DMSO);
(d) mixing, agitating, stirring, vortexing, or sonicating the resulting mixture until it is homogenous, optionally with concurrent heating;
(e) optionally filtering the resulting mixture, e.g., to remove undissolved matter;
(f) optionally sterilizing the resulting mixture; and
(g) optionally filling the resulting composition into vials or pre-filled syringes;
   wherein the Process does not result in the formation of polymeric microspheres.

In a particular embodiment of the sixth aspect, the present disclosure provides Process 3 wherein the steps comprise:
(a) filling tetradeuterolumateperone, in free or pharmaceutically acceptable salt form (e.g., tosylate salt form) into a vial or a chamber of a dual-compartment syringe, optionally with at least one nonaqueous (e.g., organic) solvent (e.g., N-methyl-2-pyrrolidone or DMSO);
(b) dissolving at least one of a poly(lactic acid) polymer, a poly(glycolic acid) polymer, a poly(lactic-co-glycolic acid) copolymer, or a polyester-poly(ethylene glycol) copolymer, as described in any embodiments hereinbefore described in least one nonaqueous (e.g., organic) solvent (e.g., N-methyl-2-pyrrolidone or DMSO), and adding the resulting solution to vials or chamber of a dual-compartment syringe;
(c) optionally sterilizing the filled vials or syringes; and
(d) packaging the vials or syringes, e.g., in a kit, for distribution to patients;
   preferably wherein mixing the contents of the vials of steps (a) and (b), or the syringe compartments of steps (a) and (b), generates a homogenous or suspension for injection.

In a seventh aspect, the present disclosure provides a method (Method 1) for the treatment or prophylaxis of a disease or disorder involving or mediated by the 5-HT_{2A} receptor, serotonin transporter (SERT), and/or dopamine D1/D2 receptor signaling pathways, comprising administering to a patient in need thereof the Composition 1, or any of 1.1-1.145. In further embodiments, the present disclosure provides:
1.1 Method 1, wherein the disease or disorder is selected from obesity, anorexia, bulimia, depression (including major depressive disorder (MDD), acute depression, post-traumatic depression), anxiety (including acute anxiety, panic disorders, phobias, social anxiety disorder, or social withdrawal), psychosis (including acute psychosis), schizophrenia (including residual symptoms of schizophrenia, such as positive and/or negative symptoms of schizophrenia), obsessive-compulsive disorder, sexual disorders, migraine, attention deficit disorder, attention deficit hyperactivity disorder, sleep disorders, conditions associated with cephalic pain, anger disorders, agitation (including acute agitation), dementia (including Alzheimer's Disease and Parkinson's dementia), gastrointestinal disorders such as dysfunction of gastrointestinal tract motility, and bipolar disorder (e.g., bipolar depression);
1.2 Method 1, wherein the disease or disorder is selected from schizophrenia, bipolar depression, anxiety, agitation, and dementia;
1.3 Method 1, wherein the disease or disorder is schizophrenia (e.g., negative symptoms of schizophrenia);
1.4 Method 1, wherein the disease or disorder is depression (e.g., bipolar depression, bipolar I disorder, or bipolar II disorder);
1.5 Method 1.4, wherein the depression is characterized by major depressive episodes (e.g., major depressive episodes associated with major depressive disorder, bipolar depression, bipolar I disorder, or bipolar II disorder);
1.6 Method 1.4 or 1.5, wherein treatment is adjunct to oral administration of valproate (e.g., valproic acid or sodium valproate) or adjunct to oral administration of an antidepressant agent (e.g., a selective serotonin reuptake inhibitor);
1.7 Method 1, wherein the disease or disorder is anxiety (e.g., general anxiety disorder);
1.8 Method 1, wherein the disease or disorder is bipolar mania (e.g., manic episodes associated with bipolar disorder, e.g., associated with bipolar I disorder);
1.9 Method 1, wherein the disease or disorder is autism spectrum disorder;
1.10 Method 1.9, wherein the disease or disorder is irritability in autism spectrum disorder;
1.11 Method 1, or any of 1.1-1.10, wherein the composition is administered by intramuscular injection;
1.12 Method 1, or any of 1.1-1.10, wherein the composition is administered by subcutaneous injection;
1.13 Method 1, or any of 1.1-1.12, wherein the composition is administered as a single injection once per week, once every 2 weeks, once every 3 weeks, once every 4 weeks, once every 6 weeks, once every 2 months, once every 3 months, once every 4 months, once every 5 months, or once every 6 months, e.g., once per month;
1.14 Method 1, or any of 1.1-1.13, wherein administration of a single injection of the composition provides a plasma concentration of lumateperone of at least 5 ng/ml, e.g., at least 10 ng/mL or at least 20 ng/mL, or 5-30 ng/ml, or 10-30 ng/ml, or 5-20 ng/mL, or 10-20 ng/mL, for a period of at least 4 weeks, e.g., at least 6 weeks, or at least 8 weeks, or at least 10 weeks, or at least 12 weeks;
1.15 Method 1, or any of 1.1-1.14, wherein administration of a single injection of the composition (e.g., a 100 mg dose), provides a steady-state plasma concentration of lumateperone of not more than 60 ng/ml, e.g., not more than 55 ng/ml, or not more than 50 ng/mL, or not more than 45 ng/ml, or not more than 40 ng/ml;
1.16 Method 1, or any of 1.1-1.14, wherein administration of a single injection of the composition (e.g., a 100 mg dose), provides a steady-state plasma concentration of lumateperone of 2.5-30 ng/ml, e.g., 2.5 to 25 ng/mL, or 2.5 to 20 ng/mL, or 2.5 to 15 ng/ml or 2.5 to 10 ng/ml, or 2.5 to 5 ng/ml;
1.17 Method 1, or any of 1.1-1.16, wherein the dose administered in a single injection is 50 to 200 mg of lumateperone, measured as the free base equivalent, e.g., 50 to 100 mg, or 100 to 150 mg, or 150 to 200 mg, 50 to 75 mg, or 75 to 125 mg, or 125 to 175 mg, or 75 to 150 mg, or 80 to 140 mg, or 90 to 130 mg, or 100 to 120 mg, or 100 to 110 mg;
1.18 Method 1, or any of 1.1-1.16, wherein the dose administered in a single injection is 200 to 600 mg of lumateperone, measured as the free base equivalent, e.g., 200 to 500 mg, or 200 to 400 mg, or 200 to 300 mg, 250 to 500 mg, or 250 to 400 mg, or 250 to 300 mg, or 300 to 500 mg, or 300 to 400 mg, or 300 to 350 mg, or 400 to 500 mg, or 400 to 450 mg, or about 100 mg, or about 200 mg, or about 300 mg, or about 400 mg, or about 500 mg, or about 600 mg;
1.19 Method 1, or any of 1.1-1.18, wherein the patient was previously treated with oral lumateperone (e.g., the patient is switched from oral lumateperone to long-acting injectable lumateperone according to Composition 1 et seq.).

In an eighth aspect, the present disclosure provides a method (Method 2) for the treatment or prophylaxis of a disease or disorder involving or mediated by the 5-HT_{2A} receptor, serotonin transporter (SERT), and/or dopamine D1/D2 receptor signaling pathways, comprising administering to a patient in need thereof the Composition 2, or any of 2.1-2.145. In further embodiments, the present disclosure provides:
2.1. Method 2, wherein the disease or disorder is selected from obesity, anorexia, bulimia, depression (including major depressive disorder (MDD), acute depression, post-traumatic depression), anxiety (including acute anxiety, panic disorders, phobias, social anxiety disorder, or social withdrawal), psychosis (including acute psychosis), schizophrenia (including residual symptoms of schizophrenia, such as positive and/or negative symptoms of schizophrenia), obsessive-compulsive disorder, sexual disorders, migraine, attention deficit disorder, attention deficit hyperactivity disorder, sleep disorders, conditions associated with cephalic pain, anger disorders, agitation (including acute agitation), dementia (including Alzheimer's Disease and Parkinson's dementia), gastrointestinal disorders such as dysfunction of gastrointestinal tract motility, and bipolar disorder (e.g., bipolar depression);
2.2. Method 2, wherein the disease or disorder is selected from schizophrenia, bipolar depression, anxiety, agitation, and dementia;
2.3. Method 2, wherein the disease or disorder is schizophrenia (e.g., negative symptoms of schizophrenia);
2.4. Method 2, wherein the disease or disorder is depression (e.g., bipolar depression, bipolar I disorder, or bipolar II disorder);
2.5. Method 2.4, wherein the depression is characterized by major depressive episodes (e.g., major depressive episodes associated with major depressive disorder, bipolar depression, bipolar I disorder, or bipolar II disorder);
2.6. Method 2.4 or 2.5, wherein treatment is adjunct to oral administration of valproate (e.g., valproic acid or sodium valproate) or adjunct to oral administration of an antidepressant agent (e.g., a selective serotonin reuptake inhibitor);
2.7. Method 2, wherein the disease or disorder is anxiety (e.g., general anxiety disorder);
2.8. Method 2, wherein the disease or disorder is bipolar mania (e.g., manic episodes associated with bipolar disorder, e.g., associated with bipolar I disorder);
2.9. Method 2, wherein the disease or disorder is autism spectrum disorder;
2.10. Method 2.9, wherein the disease or disorder is irritability in autism spectrum disorder;
2.11. Method 2, or any of 2.1-2.10, wherein the composition is administered by intramuscular injection;
2.12. Method 2, or any of 2.1-2.10, wherein the composition is administered by subcutaneous injection;
2.13. Method 2, or any of 2.1-2.12, wherein the composition is administered as a single injection once per week, once every 2 weeks, once every 3 weeks, once every 4 weeks, once every 6 weeks, once every 2 months, once every 3 months, once every 4 months, once every 5 months, or once every 6 months, e.g., once per month;
2.14. Method 2, or any of 2.1-2.13, wherein administration of a single injection of the composition provides a plasma concentration of deuterolumateperone of at least 5 ng/mL, e.g., at least 10 ng/ml or at least 20 ng/mL, or 5-30 ng/ml, or 10-30 ng/ml, or 5-20 ng/mL, or 10-20 ng/mL, for a period of at least 4 weeks, e.g., at least 6 weeks, or at least 8 weeks, or at least 10 weeks, or at least 12 weeks;
2.15. Method 2, or any of 2.1-2.14, wherein administration of a single injection of the composition (e.g., a 100 mg dose), provides a steady-state plasma concentration of deuterolumateperone of not more than 60 ng/ml, e.g., not more than 55 ng/mL, or not more than 50 ng/mL, or not more than 45 ng/ml, or not more than 40 ng/ml;
2.16. Method 2, or any of 2.1-2.14, wherein administration of a single injection of the composition (e.g., a 100 mg dose), provides a steady-state plasma concentration of deuterolumateperone of 2.5-30 ng/ml, e.g., 2.5 to 25 ng/mL, or 2.5 to 20 ng/mL, or 2.5 to 15 ng/ml or 2.5 to 10 ng/ml, or 2.5 to 5 ng/ml;
2.17. Method 2, or any of 2.1-2.16, wherein the dose administered in a single injection is 50 to 200 mg of deuterolumateperone, measured as the free base equivalent, e.g., 50 to 100 mg, or 100 to 150 mg, or 150 to 200 mg, 50 to 75 mg, or 75 to 125 mg, or 125 to 175 mg, or 75 to 150 mg, or 80 to 140 mg, or 90 to 130 mg, or 100 to 120 mg, or 100 to 110 mg;
2.18. Method 2, or any of 2.1-2.16, wherein the dose administered in a single injection is 200 to 600 mg of deuterolumateperone, measured as the free base equivalent, e.g., 200 to 500 mg, or 200 to 400 mg, or 200 to 300 mg, 250 to 500 mg, or 250 to 400 mg, or 250 to 300 mg, or 300 to 500 mg, or 300 to 400 mg, or 300 to 350 mg, or 400 to 500 mg, or 400 to 450 mg, or about 100 mg, or about 200 mg, or about 300 mg, or about 400 mg, or about 500 mg, or about 600 mg;
2.19. Method 2, or any of 2.1-2.18, wherein the patient was previously treated with oral lumateperone or deuterolumateperone (e.g., the patient is switched from oral lumateperone or deuterolumateperone to long-acting injectable deuterolumateperone according to Composition 2 et seq.).

In a ninth aspect, the present disclosure provides a method (Method 3) for the treatment or prophylaxis of a disease or disorder involving or mediated by the 5-HT_{2A} receptor, serotonin transporter (SERT), and/or dopamine D1/D2 receptor signaling pathways, comprising administering to a patient in need thereof the Composition 3, or any of 3.1-3.145. In further embodiments, the present disclosure provides:
3.1. Method 3, wherein the disease or disorder is selected from obesity, anorexia, bulimia, depression (including major depressive disorder (MDD), acute depression, post-traumatic depression), anxiety (including acute anxiety, panic disorders, phobias, social anxiety disorder, or social withdrawal), psychosis (including acute psychosis), schizophrenia (including residual symptoms of schizophrenia, such as positive and/or negative symptoms of schizophrenia), obsessive-compulsive disorder, sexual disorders, migraine, attention deficit disorder, attention deficit hyperactivity disorder, sleep disorders, conditions associated with cephalic pain, anger disorders, agitation (including acute agitation), dementia (including Alzheimer's Disease and Parkinson's dementia), gastrointestinal disorders such as dysfunction of gastrointestinal tract motility, and bipolar disorder (e.g., bipolar depression);
3.2. Method 3, wherein the disease or disorder is selected from schizophrenia, bipolar depression, anxiety, agitation, and dementia;
3.3. Method 3, wherein the disease or disorder is schizophrenia (e.g., negative symptoms of schizophrenia);
3.4. Method 3, wherein the disease or disorder is depression (e.g., bipolar depression, bipolar I disorder, or bipolar II disorder);
3.5. Method 3.4, wherein the depression is characterized by major depressive episodes (e.g., major depressive episodes associated with major depressive disorder, bipolar depression, bipolar I disorder, or bipolar II disorder);
3.6. Method 3.4 or 3.5, wherein treatment is adjunct to oral administration of valproate (e.g., valproic acid or sodium valproate) or adjunct to oral administration of an antidepressant agent (e.g., a selective serotonin reuptake inhibitor);
3.7. Method 3, wherein the disease or disorder is anxiety (e.g., general anxiety disorder);
3.8. Method 3, wherein the disease or disorder is bipolar mania (e.g., manic episodes associated with bipolar disorder, e.g., associated with bipolar I disorder);
3.9. Method 3, wherein the disease or disorder is autism spectrum disorder;
3.10. Method 3.9, wherein the disease or disorder is irritability in autism spectrum disorder;
3.11. Method 3, or any of 3.1-3.10, wherein the composition is administered by intramuscular injection;
3.12. Method 3, or any of 3.1-3.10, wherein the composition is administered by subcutaneous injection;
3.13. Method 2, or any of 3.1-3.12, wherein the composition is administered as a single injection once per week, once every 2 weeks, once every 3 weeks, once every 4 weeks, once every 6 weeks, once every 2 months, once every 3 months, once every 4 months, once every 5 months, or once every 6 months, e.g., once per month;
3.14. Method 3, or any of 3.1-3.13, wherein administration of a single injection of the composition provides a plasma concentration of tetradeuterolumateperone of at least 5 ng/mL, e.g., at least 10 ng/mL or at least 20 ng/ml, or 5-30 ng/ml, or 10-30 ng/ml, or 5-20 ng/mL, or 10-20 ng/mL, for a period of at least 4 weeks, e.g., at least 6 weeks, or at least 8 weeks, or at least 10 weeks, or at least 12 weeks;
3.15. Method 3, or any of 3.1-3.14, wherein administration of a single injection of the composition (e.g., a 100 mg dose), provides a steady-state plasma concentration of tetradeuterolumateperone of not more than 60 ng/ml, e.g., not more than 55 ng/ml, or not more than 50 ng/mL, or not more than 45 ng/mL, or not more than 40 ng/ml;
3.16. Method 3, or any of 3.1-3.14, wherein administration of a single injection of the composition (e.g., a 100 mg dose), provides a steady-state plasma concentration of tetradeuterolumateperone of 2.5-30 ng/mL, e.g., 2.5 to 25 ng/mL, or 2.5 to 20 ng/ml, or 2.5 to 15 ng/mL or 2.5 to 10 ng/mL, or 2.5 to 5 ng/ml;
3.17. Method 3, or any of 3.1-3.16, wherein the dose administered in a single injection is 50 to 200 mg of tetradeuterolumateperone, measured as the free base equivalent, e.g., 50 to 100 mg, or 100 to 150 mg, or 150 to 200 mg, 50 to 75 mg, or 75 to 125 mg, or 125 to 175 mg, or 75 to 150 mg, or 80 to 140 mg, or 90 to 130 mg, or 100 to 120 mg, or 100 to 110 mg;
3.18. Method 3, or any of 3.1-3.16, wherein the dose administered in a single injection is 200 to 600 mg of tetradeuterolumateperone, measured as the free base equivalent, e.g., 200 to 500 mg, or 200 to 400 mg, or 200 to 300 mg, 250 to 500 mg, or 250 to 400 mg, or 250 to 300 mg, or 300 to 500 mg, or 300 to 400 mg, or 300 to 350 mg, or 400 to 500 mg, or 400 to 450 mg, or about 100 mg, or about 200 mg, or about 300 mg, or about 400 mg, or about 500 mg, or about 600 mg;
3.19. Method 3, or any of 3.1-3.18, wherein the patient was previously treated with oral lumateperone or deuterolumateperone (e.g., the patient is switched from oral lumateperone or deuterolumateperone to long-acting injectable tetradeuterolumateperone according to Composition 3 et seq.).

The present disclosure further provides Composition 1, et seq., for use in the treatment of a disease or disorder according to any of Method 1, et seq., and for use in the manufacture of a medicament for the treatment of a disease or disorder according to any of Method 1, et seq.

The present disclosure further provides Composition 2, et seq., for use in the treatment of a disease or disorder according to any of Method 2, et seq., and for use in the manufacture of a medicament for the treatment of a disease or disorder according to any of Method 2, et seq.

The present disclosure further provides Composition 3, et seq., for use in the treatment of a disease or disorder according to any of Method 3, et seq., and for use in the manufacture of a medicament for the treatment of a disease or disorder according to any of Method 3, et seq.

The words “treatment” and “treating” are to be understood accordingly as embracing prophylaxis and treatment or amelioration of symptoms of disease and/or treatment of the cause of the disease. In particular embodiments, the words “treatment” and “treating” refer to prophylaxis or amelioration of symptoms of the disease.

The term “patient” may include a human or non-human patient.

Methods of synthesizing lumateperone, deuterolumateperone, tetradeuterolumateperone, and related compounds, and salts and co-crystals thereof, are known in art, and include the methods disclosed in U.S. Pat. Nos. 6,552,017, 6,548,493, 7,071,186, 7,183,282, 7,238,690, 8,309,722, 8,779,139; 9,315,504, 9,751,883, 10,077,267, 10,221,176, 10,464,938, 10,597,394, 10,597,395, 10,654,854, 10,688,097, 10,899,762, 11,014,925, 11,066,407, 11,096,944, RE39,679, and U.S. RE39,680; and US 2020/0102309, US 2020/0157100, and US 2020/0247805, the contents of each of which are incorporated by reference in their entirety.

Isolation or purification of the diastereomers of the Compounds of the Invention may be achieved by conventional methods known in the art, e.g., column purification, preparative thin layer chromatography, preparative HPLC, crystallization, trituration, simulated moving beds and the like.

The pharmaceutically acceptable salts of lumateperone or deuterolumateperone or tetradeuterolumateperone can be synthesized from the parent compound, which contains basic moieties, by reaction with a suitable acid, by conventional chemical methods. Generally, such salts can be prepared by reacting the free base forms of these compounds with a stoichiometric amount of the appropriate acid in water or in an organic solvent, or in a mixture of the two; generally, non-aqueous media like ether, ethyl acetate, ethanol, isopropanol, or acetonitrile are preferred.

Dosages employed in practicing the present disclosure will of course vary depending, e.g., on the particular disease or condition to be treated, the particular active compounds used, the mode of administration, and the therapy desired. Unless otherwise indicated, an amount of an active compound for administration (whether administered as a free base or as a salt form) refers to or is based on the amount of the compound in free form (i.e., the calculation of the amount is based on the amount of active moiety in free form, not taking into account the weight of the counter ion in the case of a salt).

For the avoidance of doubt, any disclosure of a numerical range, e.g., “up to X” amount is intended to include the upper numerical limit X. Therefore, a disclosure of “up to 60 mg” is intended to include 60 mg.

Unless indicated otherwise, all percentages disclosed herein are percentages by weight.

### Example 1: Long Acting Injectable Composition Development

It is desired to provide a solvent-based (i.e., non-microsphere) long acting injectable composition which provides for at least 1-month of in vitro release of drug, preferably maintaining an optimum plasma drug concentration of 10 to 20 ng/mL with less than 5% net drug release in the first 8 hours (i.e., no “dose-dumping”), which has a viscosity enabling it to be administered via 25 gauge needle, and stability for at least 6 months of storage.

N-methyl-2-pyrrolidone (NMP) is chosen as the solvent for initial studies due to its biocompatibility. Several polymers are evaluated as carriers for the depot, as shown in the following table:

| Polymer Type | Viscosity (dL/g) | Degradation Time | |
|---|---|---|---|
| PLGA 50:50 (acid) | 0.16-0.24 | | |
| PLGA 50:50 (acid) | 0.45-0.60 | <3 | months |
| PLGA 50:50 (ester) | 0.45-0.60 | <3 | months |
| PLGA 50:50 (ester) | 0.61-0.74 | <3 | months |
| PLGA 50:50 (ester) | 0.61-0.74 | <3 | months |
| PLGA 65:35 (acid) | 0.32-0.44 | <5 | months |
| PLGA 75:25 (ester) | 0.8-1.2 | <6 | months |
| PLGA 75:25 (acid) | 0.32-0.44 | <6 | months |
| PLGA 75:25 (ester) | 0.32-0.44 | <6 | months |
| PLGA 75:25 (ester) | 0.50-0.70 | <6 | months |
| PLGA 75:25 (ester) | 0.71-1.0 | <6 | months |
| PLGA 75:25 (ester) | 0.9-1.3 | | |
| PLA (acid) | 0.25-0.35 | <6 | months |
| PLA (ester) | 0.25-0.35 | <6 | months |
| PLA (ester) | 0.55-0.75 | <6 | months |

| | |
|---|---|
| PLA (ester) | 0.30-0.35 |
| PLA (ester) | 0.18-0.24 |
| PLA (acid) | 0.30-0.35 |
| PLA (acid) | 0.15-0.19 |
| PLGA 75:25 (acid) | 0.15-0.25 |
| PLGA 75:25 (acid) | 0.30-0.40 |
| PLGA 75:25 (acid) | 0.35-0.45 |
| PLGA 80:20 (acid) | 0.25-0.29 |

Injection time is one of the most critical factors for injectable formulation acceptance. If injection takes too long, it will be very undesirable for both patients and medical personnel. Preferably, injection of a sufficient dose takes less than 10 minutes, more preferably less than 5 minutes, or less than 3 minutes, or less than 1 minute. The viscosity (e.g., inherent viscosity) of the polymer used in the formulation is one of the major determinants of overall formulation viscosity, although the choice of solvent and concentration of polymer and active ingredient are also factors, as well as the gauge of the needle used for injection. It is generally found that polymers with a viscosity above about 0.70 dL/g were too viscous for injection using a 23 Gauge needle. Medium viscosity polymers (e.g., 0.45 to 0.70 dl/g), could be injected using a 23 Gauge needle, but long administration times were sometimes required, for example, from 35 minutes to 70 minutes for a 1 mL volume. Polymers having a viscosity under 0.45 dL/g could be more easily injected (e.g., less than 10 minutes). For polymers of highest viscosity, the injection time was 1.5-2 hours for only a 0.1 to 0.3 mL volume using a 23 Gauge needle. Even with a 20 Gauge needle, such viscous polymers required 30 minutes to an hour for injecting 0.1 to 0.5 mL volumes.

It is desirable to develop a long-acting injectable composition which, after injection, provides a relatively steady rate of drug release. Several compositions are developed and tested in rats (% values are w/w), using lumateperone free base as the active, N-methyl-2-pyrrolidone as the solvent, and various PLA and PLGA polymers:

| Formula | | | | | Dose |
|---|---|---|---|---|---|
| No. | Luma. | Polymer | NMP | Polymer Type | (mg/kg) |
| 1 | 15% | 38% | 47% | PLGA 50:50 | 60 |
| 2 | 12% | 31% | 57% | PLGA 65:35 | 60 |
| 3 | 12% | 33% | 55% | PLGA 75:25 | 65 |
| 4 | 30% | 30% | 40% | PLGA 75:25 | 162 |
| 4 | 20% | 30% | 50% | PLGA 75:25 | 108 |
| 5 | 20% | 30% | 50% | PLA | 108 |
| 6 | 20% | 30% | 50% | PLA | 108 |
| 7 | 20% | 30% | 50% | PLGA 75:25 | 108 |
| 8 | 20% | 30% | 50% | PLGA 75:25 | 108 |
| 9 | 30% | 30% | 50% | PLA | 162 |
| 10 | 20% | 30% | 40% | PLA | 108 |
| 11 | 20% | 30% | 50% | PLGA 50:50 | 108 |
| 12 | 20% | 30% | 50% | PLGA 75:25 | 108 |
| 13 | 20% | 30% | 50% | PLGA 75:25 | 108 |
| 14 | 20% | 30% | 50% | PLGA 75:25 | 108 |
| 15 | 20% | 30% | 50% | PLGA 80:20 (acid) | tbd |
| | | | | (iv 0.25-0.29) | |
| 16 | 30% | 27% | 43% | PLGA 80:20 (acid) | tbd |
| | | | | (iv 0.25-0.29) | |
| 17 | 20% | 30% | 50% | PLA (acid) (iv 0.15-0.19) | tbd |
| 18 | 25% | 33% | 42% | PLA (acid) (iv 0.15-0.19) | tbd |

The compositions are administered in a rat model. Male Sprague-Dawley rats weighing at least 300 grams are issued. Six animals are used in each test group based on random assignment. Animals are initially acclimated to their surroundings for 2 days, and are housed one per cage. Water is offered ad libitum, but food is withheld for at least 12 hours prior to dosing. The test compositions are administered by subcutaneous injection of a volume of 0.45 mL/kg with each test composition having a concentration of 200 mg lumateperone free base per gram of composition. Animals have blood drawn at 1, 24, 48, 72, 96, 120, 144, 168, 240, 336, 432, 504, 576, 648, 720, 816, 888, 1032, 1128, 1200, 1320, and 1440 hours, post-dosing. Plasma is isolated and kept in cold storage until later analysis, according to standard laboratory procedures.

It is found that using PLGA polymers with lower lactic/glycolic ratios generally results in more rapid initial release of drug, which results in excessive blood plasma levels within the first 5-10 days, and insufficient blood plasma levels later. For example, comparing Formula 1 (PLGA 50:50) to Formula 2 (PLGA 65:35), there is substantially more initial release of lumateperone from Formula 1, with the depot being exhausted within 15 days. In contrast, Formula 2 continued to release drug for more than 20 days.

It is unexpectedly found that the flattest and longest lasting release profile is provided by Formulations 3, 5, and 6. While only Formulations 4, 5 and 6 continue to provide a plasma drug concentration over 5 ng/ml at 27 days, Formulation 4 (having the highest dose) also provides a very large peak plasma concentration of close to 150 ng/ml at days 4, 5 and 6, whereas Formulations 5 and 6 provide a peak plasma concentration of only about 60 ng/ml at days 7 and 8.

Comparing Formulations 3, 4, and 5, which have the same polymer system but different lumateperone loading levels, it is found that the 108 mg/kg and 162 mg/kg formulations provide similar plasma concentration levels up through about day 14 (both showing a high peak around day 7), at which point plasma levels drop rapidly for the 108 mg/kg formulation, but remain steadier for the higher loading formulation. In contrast at the lowest 65 mg/kg loading, a relatively flat profile is obtained, with a shallow peak near day 10 and plasma levels above 5 mg/mL through day 24. This shows that the relationship between plasma profile and loading is not linear nor easily predictable, and it suggests that it might be more favorable to provide a composition with a lower loading but higher net depot size versus a test depot with a higher loading.

While a significant difference in plasma profile is not observed between formulations based on the same polymer with acid versus ester end groups, it is found that the polymers with acid end groups degrade more rapidly in vivo.

Overall, the data suggests that compositions similar to Formulation 6 would be suitable for monthly dosing, while compositions similar to Formulation 8 would be suitable for biweekly dosing.

### Example 2: Long Acting Injectable Composition Human Pharmacokinetic Study

An open-label study is conducted to determine the pharmacokinetics, safety and tolerability of single doses of lumateperone free base long-acting Injectable formulations in patients with schizophrenia. The primary objective is to determine the pharmacokinetic profile of lumateperone long-acting formulations after single intramuscular injections, and to assess the safety and tolerability of lumateperone long-acting injectable formulations after single intramuscular injections. In addition, an exploratory objection is to determine the effect of lumateperone long-acting injectable on symptoms of depression as measured by the Calgary Depression Scale for Schizophrenia (CDSS).

Four test formulations are used in the study, corresponding to Formulations 15, 16, 17, and 18, of Example 1.

Patients will be screened no more than 21 days prior to Day 1. Patients will be enrolled in one of up to three sequential study parts, and 1 of up to 8 cohorts. Each cohort will consist of approximately 8 patients. Part A will consist of up to 4 cohorts, Part B of up to 2 cohorts, and Part C of up to 2 cohorts. The three study parts will be enrolled sequentially, starting with Part A.

Within each study part, cohorts may be enrolled in parallel. All patients will receive oral lumateperone tosylate for 5 days (Day 1 through Day 5), followed by a 5-day washout of oral lumateperone (Day 6 through Day 10), followed by a single IM dose of lumateperone free base LAI on Day 11. Patients will be admitted to the study center on Day-1 and eligibility will be confirmed.

Patients enrolled in Part A, will be randomized on Day 1 to one of four cohorts as shown below. Part A:
(a) Cohort A1: multiple, once daily, oral administration of 42 mg lumateperone tosylate followed by a single IM dose of 100 mg lumateperone LAI formulation 1 (F1)
(b) Cohort A2: multiple, once daily, oral administration of 42 mg lumateperone tosylate followed by a single IM dose of 100 mg lumateperone LAI formulation 2 (F2)
(c) Cohort A3: multiple, once daily, oral administrations of 42 mg lumateperone tosylate followed by a single IM dose of 100 mg lumateperone LAI formulation 3 (F3)
(d) Cohort A4: multiple, once daily, oral administration of 42 mg lumateperone tosylate followed by a single IM dose of X mg lumateperone LAI formulation 4 (F4)

Upon completion of Part A and after evaluation of safety and pharmacokinetics (PK), one or three LAI formulations will be chosen for evaluation in Part B at doses higher than those administered in Part A. Patients enrolled in Part B, will be randomized on Day 1 to one of two cohorts as shown below. Part B:
(a) Cohort B1: multiple, once daily, oral administration of 42 mg lumateperone followed by a single IM dose of 200 mg lumateperone LAI formulation chosen from Part A
(b) Cohort B2: multiple, once daily, oral administration of 42 mg lumateperone followed by a single IM dose of 200 mg lumateperone LAI formulation chosen from Part A

Upon completion of Part B and following evaluation of safety and PK, one or two LAI formulations will be chosen for evaluation in Part C, at doses higher than those administered in Part A. Patients enrolled in Part C, will be randomized on Day 1 to one of two cohorts as shown below. Part C:
(a) Cohort C1: multiple, once daily, oral administration of 42 mg lumateperone followed by a single IM dose of 400 mg lumateperone LAI formulation chosen from Part B
(b) Cohort C2: multiple, once daily, oral administration of 42 mg lumateperone followed by a single IM dose of 400 mg lumateperone LAI formulation chosen from Part B

The planned doses proposed in Parts B and C may be adjusted upon review of safety and PK data in the preceding study part but dose escalation in each study part will not exceed 3-fold of the dose evaluated in the preceding part. The highest administered LAI IM dose in the study will not exceed 400 mg.

For each cohort, a single sentinel patient will be evaluated first, followed by the remaining patients in the cohort after the safe discharge on Day 25 of the first patient.

From 4 to 7 days prior to Day-1, eligible patients must wash off current antipsychotic therapy (except lumateperone). On Day-1, eligible patients will be admitted to a study center. Oral lumateperone (42 mg) will be administered under fasted conditions once daily in the morning for 5 days (Days 1 to 5) followed by a washout period of 5 days (Days 6 to 10). A single IM injection of LAI will be administered on the morning of Day 11 and patients will be monitored for at least 14 days while inpatient

Patients may be discharged on or after Day 25 if deemed eligible to continue as outpatients based on the opinion of the Principal Investigator. Outpatient follow-up visits will be scheduled weekly for 5 weeks, beginning on Day 32 up to at least Day 60. Any patient in Part A whose plasma lumateperone concentration is not near or below the lower limit of quantitation (LLOQ), at the Day 60 visit, will be asked to return for an unscheduled visit (with the same assessments as outpatient follow-up visits) approximately every 2 weeks until the lumateperone plasma level is near or below LLOQ.

If PK data in Part A indicate that parent drug is still detectable on Day 60 and the same formulation is chosen for evaluation in Part B, then the follow-up visits may be extended in Part B, as appropriate, for all patients. Similarly, if PK data in Part B indicate that parent drug is not near or below LLOQ on Day 60 or on the last outpatient visit beyond Day 60, then follow-up visits may be extended in all patients in Part C, as appropriate, to ensure that parent plasma levels return to levels that are near or below LLOQ. If the duration of follow-up visits is not extended beyond Day 60, then individual patients in Parts B and C whose plasma lumateperone levels are not near or below LLOQ on Day 60, will be asked to return for unscheduled visits approximately every 2 weeks until their lumateperone plasma levels are near or below LLOQ.

Safety and tolerability will be assessed throughout the study.

Approximately 64 patients with stable schizophrenia are planned to be dosed in one of up to 3 study parts. In Part A, patients will be randomized to 1 of 4 cohorts (approximately 8 patients per cohort). In Part B, patients will be randomized to 1 of 2 cohorts (approximately 8 patients per cohort) or assigned to one cohort if only one formulation is selected for evaluation. In Part C, patients will be randomized to 1 of 2 cohorts (approximately 8 patients per cohort) or assigned to one cohort if only one formulation is selected for evaluation.

Enrolled patients are selected who are male or female, 18 to 50 years of age, with a clinical diagnosis of schizophrenia according to the DSM-V, who are clinically stable and free from acute exacerbation of psychosis for at least 3 months prior to screening, ad on a stable dose of antipsychotic medication, such as lumateperone, for at least 3 months prior to screening. Patients will have a CGI-S score of less than or equal to 3.

Total study duration will be approximately 12 weeks, including a Screening Period of up to 21 days prior to dosing, a 5-day inpatient oral dosing period, followed by a 15-day inpatient IM LAI lumateperone dosing period during which a single LAI dose will be administered on Day 11; and an outpatient follow-up period through Day 60. The duration of the Follow-up Period may be extended based on PK and/or safety evaluations.

Blood samples are taken from the participants as follows: (1) before first oral dosing of lumateperone tosylate on Day 1; (2) on the Day 5, at 0.25, 0.5, 1, 1.5, 2, 4, 6, 8, 12 and 24 hours post-dose; (3) before the IM administration of the LAI formulation on Day 11; (4) on Day 11, at 0.25, 0.5, 1, 1.5, 2, 4, 6, 8, 12 and 24 hours post-IM dose; and (5) every 24 hours thereafter through 336 hour post-IM dose.

Blood samples are collected, prepared for analysis and stored according to standard procedures, and are analyzed for the plasma concentration of lumateperone and its known metabolites.

We claim:

1. A nonaqueous injectable pharmaceutical composition, comprising lumateperone: 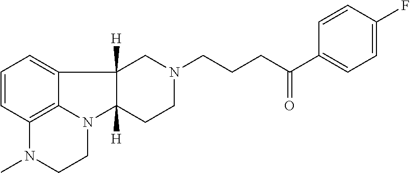 [SMILES: CN1CCN2c3c(cccc31)[C@@H]1CN(CCCC(=O)c3ccc(F)cc3)CC[C@@H]12]
or deuterolumateperone: 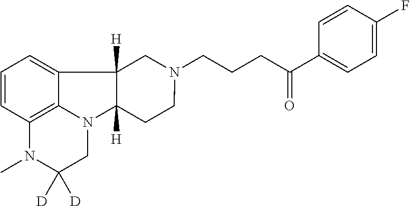 [SMILES: [2H]C1([2H])CN2c3c(cccc3N1C)[C@@H]1CN(CCCC(=O)c3ccc(F)cc3)CC[C@@H]12]
or tetradeuterolumateperone: 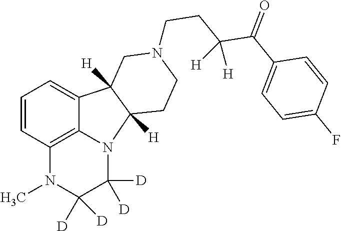 [SMILES: [2H]C1([2H])N(C)c2cccc3c2N([C@H]2CCN(CCCC(=O)c4ccc(F)cc4)C[C@@H]32)C1([2H])[2H]]
each in free or pharmaceutically acceptable salt form (e.g., in free base or tosylate salt form), wherein the lumateperone or deuterolumateperone or tetradeuterolumateperone is dissolved or suspended in a nonaqueous liquid polymeric vehicle comprising at least one nonaqueous (e.g., organic) solvent, wherein the polymeric vehicle comprises at least one of a poly(lactic acid) polymer, a poly(glycolic acid) polymer, a poly(lactic-co-glycolic acid) copolymer, and a polyester-poly(ethylene glycol) copolymer, and wherein the composition does not comprise polymeric microspheres.

2. The composition of claim 1, wherein the composition comprises lumateperone.

3. The composition of claim 1, wherein the composition comprises deuterolumateperone or tetradeuterolumateperone.

4. The composition of claim 1, wherein the composition comprises the lumateperone or deuterolumateperone or tetradeuterolumateperone in free base form.

5. The composition of claim 1, wherein the composition comprises the lumateperone or deuterolumateperone or tetradeuterolumateperone in pharmaceutically acceptable salt or co-crystal form.

6. The composition of claim 5, wherein the composition comprises the lumateperone or deuterolumateperone or tetradeuterolumateperone in tosylate salt form, e.g., in one or more of mono-tosylate salt form, di-tosylate salt form, and tri-tosylate salt form.

7. The composition of claim 1, wherein the at least one nonaqueous solvent is a pharmacologically safe water-miscible organic solvent (e.g., N-methyl-2-pyrrolidone (NMP) or DMSO) or a pharmacologically safe triglyceride oil (e.g., a vegetable oil).

8. The composition of claim 7, wherein the organic solvent is N-methyl-2-pyrrolidone (NMP).

9. The composition of claim 1, wherein the polymeric vehicle comprises a poly(lactic acid) polymer.

10. The composition of claim 1, wherein the polymeric vehicle comprises a poly(glycolic acid) polymer.

11. The composition of claim 1, wherein the polymeric vehicle comprises a poly(lactic-co-glycolic acid) copolymer.

12. The composition of claim 11, wherein the poly(lactic-co-glycolic acid) copolymer has a lactic to glycolic molar ratio of about 99:1 to 50:50, e.g., about 99:1 to 80:20, or about 99:1 to 90:10, or about 90:10 to 75:25, or about 90:10 to 80:20, or about 80:20 to 75:25, or about 75:25 to 50:50, or about 85:15 to 75:25, or about 70:30 to 60:40, or about 75:25, or about 80:20, or about 85:15, or about 90:10, or about 95:15, or about 98:2, or about 99:1.

13. The composition of claim 1, wherein the composition consists essentially of the lumateperone or deuterolumateperone or tetradeuterolumateperone (e.g., lumateperone or deuterolumateperone or tetradeuterolumateperone free base), the one or more of a poly(lactic acid) polymer, a poly(glycolic acid) polymer, and a poly(lactic-co-glycolic acid) copolymer and the solvent (e.g., N-methyl-2-pyrrolidone or DMSO).

14. The composition of claim 13, wherein the composition consists essentially of the lumateperone or deuterolumateperone or tetradeuterolumateperone free base, the poly(lactic acid) polymer and the solvent N-methyl-2-pyrrolidone.

15. The composition of claim 13, wherein the composition consists essentially of the lumateperone or deuterolumateperone or tetradeuterolumateperone free base, the poly(lactic-co-glycolic acid) copolymer (e.g., PLGA 65:35) and the solvent N-methyl-2-pyrrolidone or DMSO.

16. A single-use kit, multiple-use kit, or pre-filled dual-compartment syringe (e.g., an automatic syringe) comprising the composition of claim 1.

17. A process for the manufacture of the composition according to claim 1, wherein the process comprises the steps of:
(a) combining lumateperone or deuterolumateperone or tetradeuterolumateperone, in free or pharmaceutically acceptable salt form (e.g., tosylate salt form), with at least one nonaqueous (e.g., organic) solvent (e.g., N-methyl-2-pyrrolidone or DMSO);
(b) optionally mixing, agitating, stirring, vortexing, or sonicating the resulting mixture until it is homogenous, optionally with concurrent heating;
(c) adding at least one of a poly(lactic acid) polymer, a poly(glycolic acid) polymer, a poly(lactic-co-glycolic acid) copolymer, or a polyester-poly(ethylene glycol) copolymer, as described in any embodiments hereinbefore described, optionally in a nonaqueous (e.g., organic) solvent (e.g., N-methyl-2-pyrrolidone or DMSO);
(d) optionally mixing, agitating, stirring, vortexing, or sonicating the resulting mixture until it is homogenous, optionally with concurrent heating;
(e) optionally filtering the resulting mixture, e.g., to remove undissolved matter;
(f) optionally sterilizing the resulting mixture; and
(g) optionally filling the resulting composition into vials or pre-filled syringes (e.g., single-component syringes);
wherein the process does not result in the formation of polymeric microspheres.

18. A process for the manufacture of the composition according to claim 1, wherein the process comprises the steps of:
(a) filling lumateperone or deuterolumateperone or tetradeuterolumateperone, in free or pharmaceutically acceptable salt form (e.g., tosylate salt form) into a vial or a chamber of a dual-compartment syringe, optionally with at least one nonaqueous (e.g., organic) solvent (e.g., N-methyl-2-pyrrolidone or DMSO);
(b) dissolving at least one of a poly(lactic acid) polymer, a poly(glycolic acid) polymer, a poly(lactic-co-glycolic acid) copolymer, or a polyester-poly(ethylene glycol) copolymer, as described in any embodiments hereinbefore described in least one nonaqueous (e.g., organic) solvent (e.g., N-methyl-2-pyrrolidone or DMSO), and adding the resulting solution to vials or chamber of a dual-compartment syringe;
(c) optionally sterilizing the filled vials or syringes; and
(d) packaging the vials or syringes, e.g., in a kit, for distribution to patients;
preferably wherein mixing the contents of the vials of steps (a) and (b), or the syringe compartments of steps (a) and (b), generates a homogenous or suspension for injection, wherein the process does not result in the formation of polymeric microspheres.

19. A method for the treatment or prophylaxis of a disease or disorder involving or mediated by the 5-HT_{2A} receptor, serotonin transporter (SERT), and/or dopamine D1/D2 receptor signaling pathways, comprising administering to a patient in need thereof the composition according to claim 1.
